(19) United States
(12) Patent Application Publication
(10) Pub. No.: US 2025/0382355 A1
(43) Pub. Date: Dec. 18, 2025

(54) POLYPEPTIDES EFFECTIVE AGAINST MULTIPLE CORONAVIRUSES

(71) Applicant: MERCK SHARP & DOHME LLC, Rahway, NJ (US)

(72) Inventors: Kalyan PANDE, San Francisco, CA (US); Scott A. HOLLINGSWORTH, San Francisco, CA (US); Bernhard Hubert GEIERSTANGER, Solana Beach, CA (US); Daniel M. GORMAN, Redwood City, CA (US); David U. GRANDY, Redwood City, CA (US); Cameron L. NOLAND, San Francisco, CA (US); Arthur FRIDMAN, Lake Ariel, PA (US); Anasuya SAHA, Fremont, CA (US); Karin VROOM, San Mateo, CA (US); Lan ZHANG, Chalfont, PA (US)

(73) Assignee: MERCK SHARP & DOHME LLC, Rahway, NJ (US)

(21) Appl. No.: 19/108,781

(22) PCT Filed: Sep. 11, 2023
(86) PCT No.: PCT/US2023/032366

§ 371 Date: Mar. 5, 2025

Related U.S. Application Data
(60) Provisional application No. 63/375,583, filed on Sep. 14, 2022.

(51) Int. Cl. C07K 16/10

(52) U.S. Cl. CPC C07K 16/1003; C07K 2317/33; C07K 2317/34; C07K 2317/35; C07K 2317/569; C07K 2317/76; C07K 2317/92

(57) ABSTRACT

Disclosed are single-domain antibodies that can bind (e.g., separately) to at least two spike proteins from different coronaviruses. Additionally disclosed are polypeptides that have multiple coronavirus spike-protein binders and can neutralize (e.g., separately) at least two different coronaviruses. These single-domain antibodies and polypeptides can be used for treating coronavirus infections.

### CROSS-REFERENCE TO RELATED APPLICATIONS

This application claims the benefit of U.S. Provisional Patent Application Ser. No. 63/375,583 filed Sep. 14, 2022, the entire contents of which are incorporated by reference herein.

### REFERENCE TO SEQUENCE LISTING SUBMITTED ELECTRONICALLY

The sequence listing of the present application is submitted electronically via EFS-Web in an xml format with a file name 25568-US—PSP_SEQTXT_26082022.xml, having a creation date of Aug. 26, 2022, and a size of 214 kb. This sequence listing submitted via EFS-Web is part of the specification and is herein incorporated by reference in its entirety.

### BACKGROUND

Coronaviruses (CoVs) are a large family of viruses that infect numerous species including humans and consist of four main genera known as alpha, beta, gamma, and delta. The most significant CoV species, severe acute respiratory syndrome coronavirus 2 (SARS-CoV-2), a beta-coronavirus that emerged in China in 2019, has resulted in over 500 million cases and 15 million excess deaths (Wang et al. 2022 399:1513-1536). Other pandemic strains of CoV that have been identified include SARS-CoV and MERS, both of which have resulted in smaller but significant outbreaks with high morbidity and mortality.

Coronaviruses are single-stranded RNA (ssRNA) viruses with a large genome size and a relatively high mutation rate. Recombination events among different CoV species have been shown to occur, resulting in further genetic variability (Forni et al. 2017 25:35-48). One of the key factors driving the continued large burden of COVID-19 disease is the observed high rate of mutation of the SARS-CoV-2 virus, resulting in the emergence and rapid spread of novel viral variants capable of evading natural and vaccine-induced host immune responses. Based on systematic genomic sequencing of clinical isolates of SARS-CoV-2, there are 12 lineage groups that have been identified and 5 of them, Alpha (B.1.1.7), Beta (B.1.351), Gamma (P.1), Delta (B.1.617.2), and Omicron (B.1.1.529), have been defined as variants of concern (VOCs) by the World Health Organization. Multiple studies have reported resistance of the Omicron variant against neutralization by antibodies and serum targeting the wild type (Wuhan) strain, significantly impacting the protective efficacy of current vaccines and therapeutic antibodies (Cao et al. 2022 602:657-663, Cele et al. 2022 602:654-656, Dejnirattisai et al. 2022 399:234-236, Liu et al. 2022 602:676-681). Furthermore, the threat of continued zoonotic spillovers warrants the development of broadly reactive antiviral agents that could combat coronaviruses with pandemic potential in the future (Simpson et al. 2020 20: e108-e115).

While the vast majority of antibodies targeting the SARS-CoV-2 spike protein have been conventional immunoglobulins, several potent heavy-chain variable domains (VHHs) from camelid-derived single-domain antibodies (sdAbs) targeting the CoV-2 spike have also been reported (Esparza et al. 2020 10:22370, Schoof et al. 2020 370:1473-1479, Koenig et al. 2021 371). Several cross-reactive epitopes on the spike have been identified in the literature, including Class 3 and Class 4 epitopes on the receptor binding domain (RBD) (Barnes et al. 2020 Nature 588 (7839): 682-687; Pinto et al., 2020 Nature 583 (7815): 290-295; Yuan et al. 2020 Science 368 (6491): 630-633; Baum et al. 2020 Science 370 (6520): 1110⁻¹¹¹⁵; Wrapp et al. 2020 Cell 181 (5): 1004-1015 e15). Since VHHs are smaller compared to conventional antibodies (˜15 kDa vs ˜150 kDa), they have the potential to bind to smaller, conserved epitopes shared among different coronaviruses that conventional antibodies might not access. Moreover, VHHs have been shown to possess favorable biophysical properties and their smaller size also facilitates generation of multivalent constructs.

A broadly neutralizing coronavirus agent would be desirable not only to prevent and treat COVID-19, but also provide protection for high-risk populations against future emergent coronaviruses.

### SUMMARY

As all coronaviruses use spike proteins on the viral surface to enter the host cells, and these spike proteins share sequence and structural homology, we set out to discover cross-reactive biologic agents targeting the spike protein to block viral entry. Through llama immunization campaigns, we have identified single domain antibodies (VHHs) that are cross-reactive among multiple emergent coronaviruses (SARS-CoV, SARS-CoV-2/variants, and MERS). Importantly, a number of these antibodies show sub-nanomolar potency towards all SARS-like viruses including emergent CoV-2 variants. We identified nine distinct epitopes on the spike protein targeted by these VHHs. By engineering VHHs targeting distinct, conserved epitopes into multi-valent formats, we significantly enhanced their neutralization potencies compared to the corresponding VHH cocktails. This approach is ideally suited to address both emerging SARS-CoV-2 variants as well as potential future SARS-like coronaviruses.

In some aspects, polypeptides that bind to spike proteins from at least two different coronaviruses comprise, in N to C order, the regions framework region (FR) 1, complementarity determining region (CDR) 1, FR2, CDR2, FR3, CDR3, and FR4, wherein said CDR1 comprises the sequence of any one of SEQ ID NOs 31 to 54, said CDR2 comprises the sequence of any one of SEQ ID NOs 61 to 84, and said CDR3 comprises the sequence of any one of SEQ ID NOs 91 to 114; or wherein said CDR1, CDR2, and CDR3 respectively comprise the sequence of any one of SEQ ID NOs 31 to 54, 61 to 84, and 91 to 114 with one to three total amino acid residue mutations among themselves.

In some embodiments, said one to three residue mutations comprise at least one substitution, wherein said substitution is a conservative substitution. In some embodiments, each of said one to three residue mutations is a conservative substitution. In some embodiments, said CDR1 comprises the sequence of any one of SEQ ID NOs 31 to 54, said CDR2 comprises the sequence of any one of SEQ ID NOs 61 to 84, and said CDR3 comprises the sequence of any one of SEQ ID NOs 91 to 114. In some embodiments, the SEQ ID NOs of the sequences of said CDR1, CDR2, and CDR3 are congruent with each other in modulo 30.

In some embodiments, said FR1 comprises the sequence of any one of SEQ ID NOs 121 to 146, said FR2 comprises the sequence of any one of SEQ ID NOs 151 to 176, said FR3 comprises the sequence of any one of SEQ ID NOs 181 to 206, or said FR4 comprises the sequence of any one of SEQ ID NOs 211 to 236; or wherein said FR1, FR2, FR3, and FR4 respectively comprise the sequence of any one of SEQ ID NOs 121 to 144, 151 to 174, 181 to 204, and 211 to 234 with one to nine total residue mutations among themselves. In some embodiments, said FR1 comprises the sequence of any one of SEQ ID NOs 121 to 146, said FR2 comprises the sequence of any one of SEQ ID NOs 151 to 176, said FR3 comprises the sequence of any one of SEQ ID NOs 181 to 206, and said FR4 comprises the sequence of any one of SEQ ID NOs 211 to 236; or wherein said FR1, FR2, FR3, and FR4 respectively comprise the sequence of any one of SEQ ID NOs 121 to 144, 151 to 174, 181 to 204, and 211 to 234 with one to nine total residue mutations among themselves. In some embodiments, said one to nine residue mutations comprise at least one substitution, wherein said substitution is a conservative substitution. In some embodiments, each of said one to nine mutations is a conservative substitution. In some embodiments, said FR1 comprises the sequence of any one of SEQ ID NOs 121 to 146, said FR2 comprises the sequence of any one of SEQ ID NOs 151 to 176, said FR3 comprises the sequence of any one of SEQ ID NOs 181 to 206, and said FR4 comprises the sequence of any one of SEQ ID NOs 211 to 236. In some embodiments, the SEQ ID NOs of the sequences of said FR1, FR2, FR3, and FR4 are congruent with each other in modulo 30. In some embodiments, said regions are in said N to C order contiguously.

In some embodiments, the polypeptides comprise a sequence that has at least 90, 91, 92, 93, 94, 95, 96, 97, 98, or 99% identity with the entire sequence of any one of SEQ ID NOs 1 to 24. In some embodiments, the polypeptides comprise the sequence of any one of SEQ ID NOs 1 to 24.

In some embodiments, said different coronaviruses comprise different species selected from SARS-CoV, SARS-CoV2, and MERS-CoV. In some embodiments, said different coronaviruses consist of SARS-CoV and SARS-CoV2. In some embodiments, said different coronaviruses comprise different SARS-CoV2 variants selected from B.1.1.7, B.1.351, P.1, B.1.617.2, and B.1.1.529.

In some embodiments, the polypeptides bind (i.e., comprise a binding affinity) to each of said spike proteins with an EC50 (i.e., concentration resulting in 50% binding) value that is numerically lower than 100 nM as measured by an enzyme-linked immunosorbent (ELISA) assay. In some embodiments, the polypeptides bind (i.e., comprise a binding affinity) to each of said spike proteins with a K_{D} value that is numerically lower than 100 nM as measured by Surface Plasmon Resonance assay (SPR). In some embodiments, the polypeptides comprise said binding affinity when in a monovalent form. In some embodiments, the polypeptides are single-domain antibodies, single-chain variable fragments, antibodies, Fab fragments, F(ab′)2 fragments, Fab′ fragments, or Fv fragments. In some embodiments, the polypeptides separately inhibit infection of Vero-E6 cells by said at least two different coronaviruses with an IC50 (i.e., concentration resulting in 50% inhibition) value that is numerically lower than 100 nM.

In some aspects, single-domain antibodies comprise a CDR1 having the sequence of any one of SEQ ID NOs 31 to 54; a CDR2 having the sequence of any one of SEQ ID NOs 61 to 84; and a CDR3 having the sequence of any one of SEQ ID NOs 91 to 114, wherein the SEQ ID NOs of the sequences of said CDR1, CDR2, and CDR3 are congruent with each other in modulo 30.

In some embodiments, the single-domain antibodies comprise a sequence that has at least 90, 91, 92, 93, 94, 95, 96, 97, 98, or 99% identity with the entire sequence of any one of SEQ ID NOs 1 to 24. In some embodiments, the single-domain antibodies comprise the sequence of any one of SEQ ID NOs 1 to 24.

In some embodiments, the single-domain antibodies comprise binding affinities, when in a monovalent form, to at least two spike proteins, wherein said binding affinities are measured via SPR as K_{D} values that are numerically lower than 100 nM, wherein said at least two spike proteins are from different coronaviruses selected from SARS-CoV, SARS-CoV2, and MERS-CoV. In some embodiments, the single-domain antibodies, when in a monovalent form, bind (i.e., comprise a binding affinity) to at least two spike proteins with a K_{D} value that is numerically lower than 100 nM as measured with SPR, wherein said at least two spike proteins are from at least two different coronaviruses selected from SARS-CoV2 variants B.1.1.7, B.1.351, P.1, B.1.617.2, and B.1.1.529. In some embodiments, the single-domain antibodies, when in a monovalent form, exhibit neutralization potencies against infection of Vero-E6 cells by at least two different coronaviruses, wherein said neutralization potencies measured as IC50 values are numerically lower than 100 nM, wherein said coronaviruses are selected from SARS-CoV, SARS-CoV2, and MERS-CoV. In some embodiments, the single-domain antibodies, when in a monovalent form, separately inhibit infection of Vero-E6 cells by at least two different coronaviruses with an IC50 value that is numerically lower than 100 nM, wherein said coronaviruses are selected from SARS-CoV2 variants B.1.1.7, B.1.351, P.1, B.1.617.2, and B.1.1.529.

In some embodiments, the single-domain antibodies, when in a bivalent form (e.g., expressed as a fusion protein with an Fc, so that it forms a dimer), bind (i.e., comprise a binding affinity) to at least two spike proteins with a K_{D} value that is numerically lower than 1 nM as measured by SPR, wherein said at least two spike proteins are from different coronaviruses selected from SARS-CoV, SARS-CoV2, and MERS-CoV. In some embodiments, the single-domain antibodies, when in a bivalent form (e.g., expressed as a fusion protein with an Fc, so that it forms a dimer), bind (i.e., comprise a binding affinity) to at least two spike proteins with a K_{D} value that is numerically lower than 1 nM as measured by SPR, wherein said at least two spike proteins are from at least two different coronaviruses selected from SARS-CoV2 variants B.1.1.7, B.1.351, P.1, B.1.617.2, and B.1.1.529. In some embodiments, the single-domain antibodies, when in a bivalent form (e.g., expressed as a fusion protein with an Fc, so that it forms a dimer), separately inhibit infection of Vero-E6 cells by at least two different coronaviruses with an IC50 value that is numerically lower than 10 nM, wherein said coronaviruses are selected from SARS-CoV, SARS-CoV2, and MERS-CoV. In some embodiments, the single-domain antibodies, when in a bivalent form (e.g., expressed as a fusion protein with an Fc, so that it forms a dimer), separately inhibit infection of Vero-E6 cells by at least two different coronaviruses with an IC50 value that is numerically lower than 10 nM, wherein said coronaviruses are selected from SARS-CoV2 variants B.1.1.7, B.1.351, P.1, B.1.617.2, and B.1.1.529.

In some aspects, single-domain antibodies bind to the same epitope as any of the polypeptides or as the single-domain antibodies of any of the other embodiments.

In some embodiments, said epitope is on the N-terminal domain (NTD), S2 domain, or receptor binding domain (RBD) of a coronavirus spike protein. In some embodiments, said epitope is on the apical end of the NTD of a coronavirus spike protein. In some embodiments, said epitope comprises the sequence of any one of SEQ ID NOs 241 to 249 or comprises the sequence of residues 1176-1178 of SEQ ID NO: 240. In some embodiments, said epitope comprises any one of the sequences mentioned in the brief descriptions of FIG. 3A, FIG. 3B, and FIGS. 7A-7R. In some embodiments, said epitope is determined via hydrogen-deuterium exchange mass spectrometry.

In some aspects, polypeptides comprise two spike-protein binders each independently selected from the polypeptides or the single-domain antibodies of any of the embodiments.

In some embodiments, the polypeptides further comprise a linker between the two spike-protein binders. In some embodiments, the linker comprises 10 to 90 amino acids.

In some aspects, polypeptides comprise a first spike-protein binder, a second spike-protein binder, and a third spike-protein binder, wherein each spike-protein binder is independently selected from the polypeptides or the single-domain antibodies of any of the embodiments.

In some embodiments, the polypeptides further comprise a first linker between the first spike-protein binder and the second spike-protein binder. In some embodiments, the polypeptides further comprise a second linker between the second spike-protein binder and the third spike-protein binder. In some embodiments, the first linker comprises 10 to 30 amino acids. In some embodiments, the second linker comprises 10 to 70 amino acids.

In some embodiments, the spike-protein binders independently bind to an NTD, an S2, or an RBD of the spike protein. In some embodiments, each spike-protein binder binds to an RBD of the spike protein. In some embodiments, each spike-protein binder binds to the RBD of a different monomer of the spike protein. In some embodiments, the spike-protein binders collectively bind to an NTD, an S2, and an RBD of the spike protein. In some embodiments, the first spike-protein binder binds to an RBD, the second spike-protein binder binds to an NTD, and the third spike-protein binder binds to the S2 of the spike protein.

In some embodiments, the first spike-protein binder, the second spike-protein binder, and the third spike-protein binder respectively comprise CDR3s having the sequence of the following SEQ ID NOs: 107-91-111; 91-111-109; 107-92-114; 92-107-114; 91-107-105; 91-111-105; 91-107-111; 91-111-114; 107-91-105; 107-111-105; 107-111-109; 107-111-114; 111-105-107; 111-105-105; 105-105-107; 105-111-114; 105-105-114; 105-99-114; 113-105-114; 113-105-107; 113-111-114; 111-105-113; 113-99-114; or 113-105-113, wherein the first spike-protein binder, the second spike-protein binder, and the third spike-protein binder further comprise CDR1s having the sequence of any one of SEQ ID NOs 31 to 54, and CDR2s having the sequence of any one of SEQ ID NOs 61 to 84, wherein the SEQ ID NOs of the sequences of said CDR1, CDR2, and CDR3 are congruent with each other in modulo 30.

In some embodiments, the first spike-protein binder, the second spike-protein binder, and the third spike-protein binder respectively comprise the sequences of the following SEQ ID NOs: 17-1-21; Jan. 21, 2019; 17-2-24; Feb. 17, 2024; Jan. 17, 2015; Jan. 21, 2015; Jan. 17, 2021; Jan. 21, 2024; 17-1-15; 17-21-15; 17-21-19; 17-21-24; 21-15-17; 21-15-15; 15-15-17; 15-21-24; 15-15-24; 15-9-24; 23-15-24; 23-15-17; 23-21-24; 21-15-23; 23-9-24; or 23-15-23.

In some embodiments, the first spike-protein binder, the second spike-protein binder, and the third spike-protein binder comprise any of the constructs mentioned in Tables 6 and 7, the sequences of which can be deduced with respect to Table D, which uses the same clone names used in Tables 6 and 7. In some embodiments, the first spike-protein binder, the second spike-protein binder, and the third spike-protein binder comprise a homotrimer of any of the sequences mentioned in Table D (e.g., the VHH sequences in column 3, such as SEQ ID NO: 1 or SEQ ID NO: 2).

In some aspects, polypeptides comprise four or more spike-protein binders each independently selected from the polypeptides or the single-domain antibodies of any of the embodiments.

In some embodiments, the polypeptides separately exhibit neutralization potencies against infection of Vero-E6 cells by at least two different coronaviruses selected from SARS-CoV, SARS-CoV2, and MERS-CoV, wherein said neutralization potencies measured as IC50 values are numerically lower than those for a mixture of corresponding spike-protein binders. In some embodiments, the polypeptides separately exhibit neutralization potencies against infection of Vero-E6 cells by at least two different coronaviruses selected from SARS-CoV2 variants B.1.1.7, B.1.351, P.1, B.1.617.2, and B.1.1.529, wherein said neutralization potencies measured as IC50 values are numerically lower than those for a mixture of corresponding spike-protein binders.

In some aspects, polypeptides consist of an antigen-binding fragment of the polypeptides or the single-domain antibodies of any of the embodiments. In some aspects, the single-domain antibodies, multimers thereof, polypeptides, and antigen-binding fragments thereof are isolated single-domain antibodies, multimers thereof, polypeptides, and antigen-binding fragments thereof.

In some aspects, polypeptides compete with the polypeptides or the single-domain antibodies of any of the embodiments for binding to a coronavirus spike protein.

In some aspects, compositions comprise the polypeptides or the single-domain antibodies of any of the embodiments and a pharmaceutically acceptable carrier. In some aspects, kits comprise such compositions. In some embodiments, the compositions are contained within a vial or injection device. In some embodiments, the kits further comprise a second therapeutic agent or vaccine.

In some aspects, isolated nucleic acids (e.g., DNA) encode the polypeptides or the single-domain antibodies of any of the embodiments. In some aspects, expression vectors comprise such nucleic acids. In some aspects, host cells comprise such expression vectors.

In some aspects, conjugates comprise the polypeptides or the single-domain antibodies of any of the embodiments, and a therapeutic agent. In some embodiments, the therapeutic agent comprises an antibody or fragment thereof, an immunomodulator, a hormone, a cytotoxic agent, an enzyme, a radionuclide, an antibody conjugated to at least one immunomodulator, enzyme, radioactive label, hormone, antisense oligonucleotide, or cytotoxic agent, or a combination thereof.

In some aspects, conjugates comprise polypeptides or the single-domain antibodies of any of the embodiments, and a half-life extender. In some embodiments, the half-life extender comprises a heavy chain constant domain or a crystallizable fragment domain.

In some aspects, methods for producing the polypeptides or the single-domain antibodies of any of the embodiments comprise cultivating the host cell of any embodiment in a medium under conditions suitable for expression of the polypeptide or single-domain antibody by the host cell; and isolating the polypeptide or single-domain antibody from the medium.

In some aspects, methods of neutralizing a coronavirus in a sample comprise contacting the sample with an effective amount of the polypeptides or the single-domain antibodies of any of the embodiments. In some embodiments, the coronavirus is SARS-CoV, SARS-CoV2, or MERS-CoV. In some embodiments, the coronavirus is a SARS-CoV2 variant selected from B.1.1.7, B.1.351, P.1, B.1.617.2, and B.1.1.529.

In some aspects, methods of treating a coronavirus infection in a subject comprise administering to a subject in need thereof an effective amount of one or more of the polypeptides or the single-domain antibodies of any of the embodiments. In some embodiments, the coronavirus infection is of SARS-CoV, SARS-CoV2, or MERS-CoV. In some aspects, methods of treating a coronavirus disease in a subject comprise administering to a subject in need thereof an effective amount of a composition comprising one or more of the polypeptides or the single-domain antibodies of any of the embodiments. In some embodiments, the coronavirus disease is SARS, MERS, or COVID-19.

In some aspects, the polypeptides or the single-domain antibodies of any of the embodiments are for use in the treatment of a coronavirus infection. In some embodiments, the coronavirus infection is of SARS-CoV, SARS-CoV2, or MERS-CoV. In some aspects, the polypeptides or the single-domain antibodies of any of the embodiments are for use in the treatment of a coronavirus disease. In some embodiments, the coronavirus disease is SARS, MERS, or COVID-19.

In some aspects, uses of the polypeptides or the single-domain antibodies of any of the embodiments are for the treatment of a coronavirus infection. In some embodiments, the coronavirus infection is of SARS-CoV, SARS-CoV2, or MERS-CoV. In some aspects, uses of a composition comprising the polypeptides or the single-domain antibodies of any of the embodiments are for the treatment of a coronavirus disease. In some embodiments, the coronavirus disease is SARS, MERS, or COVID-19.

In some aspects, uses of the polypeptides or the single-domain antibodies of any of the embodiments are in the manufacture of a medicament for the treatment of a coronavirus infection. In some embodiments, the coronavirus infection is of SARS-CoV, SARS-CoV2, or MERS-CoV. In some aspects, uses of a composition comprising one or more of the polypeptides or the single-domain antibodies of any of the embodiments are in the manufacture of a medicament for the treatment of a coronavirus disease. In some embodiments, the coronavirus disease is SARS, MERS, or COVID-19.

### BRIEF DESCRIPTION OF THE DRAWINGS

FIG. 1A to FIG. 1D: Identification of cross-reactive VHHs following llama immunization. FIG. 1A. Llama immunization scheme using purified spike proteins from different viruses. FIG. 1B. ELISA binding results of VHHs showing cross-binding to SARS-CoV1 and SARS-CoV2 spike proteins (in monovalent and bivalent formats). FIG. 1C. Sensograms of select VHH binders to SARS-CoV2 spike protein. FIG. 1D. Table of cross-reactive VHHs to different domains (RBD, NTD, S2) within the spike protein. The structure of the SARS-CoV-2 Spike(S) protein is shown.

FIG. 2A to FIG. 2C: Pseudovirus and authentic virus neutralization. FIG. 2A. SARS-CoV and SARS-CoV-2 pseudovirus neutralization (estimated IC50 values) by 16 candidate VHHs. Std-A antibody used as a reference. FIG. 2B. Authentic SARS-CoV virus neutralization by select candidates. FIG. 2C. Authentic SARS-CoV-2 virus neutralization by select candidates.

FIG. 3A and FIG. 3B: Identification of VHH binding epitopes to guide linker length design. The epitopes of select S2 binders were determined by HDX-MS using the spike protein S2 domain. FIG. 3A. Hydrogen-deuterium exchange difference plots are shown for S3_29, 11F5 and 6A1, respectively. Sequence regions showing significant differences are LVKQLSSNFGAISSVLNDILSRLDKVEAEVQIDRLITGRLQSLQT (SEQ ID NO: 246) or amino acid (AA) 962-1006 of SEQ ID NO: 240 for S3_29; MQMAYRENGIGVTQNVL (SEQ ID NO: 247) or AA 899-916 of SEQ ID NO: 240 and EPQIITTDNFVSGNCDVVIGIVNNTVYDPLQPEL (SEQ ID NO: 248) or AA 1111-1145 of SEQ ID NO: 240 for 11F5; VVN or AA 1176-1178 of SEQ ID NO: 240 and DRLNEVAKNLNESL (SEQ ID NO: 249) or AA 1183-1197 of SEQ ID NO: 240 for 6A1. FIG. 3B. The structure of the SARS-CoV-2 Spike(S) protein is shown. The HDX mapping of the S2 targeting VHHs are shown on the same structure as above. As the region that 6A1 targets has not been resolved in a full spike protein structure (it is just beyond the end of the spike that is resolved in Cryo-EM), it is not shown in this figure.

FIG. 4A to FIG. 4E: VHH 7A9 binds a rare RBD epitope and triggers spike trimer dissociation. FIG. 4A. Crystal structure of VHH 7A9 bound to the SARS-CoV-2 RBD. FIG. 4B. Molecular interactions between VHH 7A9 and the SARS-CoV-2 RBD. FIG. 4C. SARS-CoV-2 RBD SARS-CoV-2 RBD in cartoon and transparent surface notation with the Ca carbons of residues mutated in the Omicron variant. FIG. 4D. Overlay of the SARS-CoV-2 RBD from the crystal structure onto the closed (left) or open (right) spike protein structures (PDBs: 7DF3 and 6XKL, respectively). FIG. 4E. Continuous distribution (c(s)) analyses of analytical ultracentrifugation data. From top to bottom: 7A9 VHH alone, 1E4 VHH alone, SARS-CoV-2 Spike alone, SARS-CoV-2 Spike+7A9 VHH, and SARS-CoV-2 Spike+1E4 VHH.

FIG. 5A to FIG. 5F: Multimeric VHHs enhance potency. FIG. 5A. Structure of the spike protein with a model of a homotrimeric VHH targeting RBD. FIG. 5B. Potentiation of SARS-CoV2 pseudovirus neutralization with a VHH trimer compared with a VHH monomer. FIG. 5C. Potentiation of SARS-CoV2 authentic virus neutralization with a VHH trimer compared with a VHH monomer. FIG. 5D. Structure of the spike protein with a model of a heterotrimeric VHH targeting RBD, NTD and S2. FIG. 5E. Potentiation of SARS-CoV2 pseudovirus neutralization with a VHH heterotrimer compared to a cocktail of individual VHHs. FIG. 5F. Potentiation of SARS-CoV2 authentic virus neutralization with a VHH heterotrimer compared to a cocktail of individual VHHs.

FIG. 6: Sequence coverage map obtained for spike protein S2 domain using HDX-MS.

FIGS. 7A-7R: Differential heat map of time-course H/D exchange of recombinant CoV-2 RBD alone to that of Cov-2 RBD in complex with 7A9 VHH. CoV-2 RBD showed significant reduction in deuterium uptake upon binding to the 7A9 VHH at sequences NRKRISNCVADY (SEQ ID NO: 241), which was assigned as the main binding site on CoV-2 RBD, or AA 353-364 of SEQ ID NO: 240. Moreover, there was significant reduction in deuterium sequences FTNVY (SEQ ID NO: 242) or AA 391-395 of SEQ ID NO: 240, KPFER (SEQ ID NO: 243) or AA 461-465 of SEQ ID NO: 240, ATVCGPK (SEQ ID NO: 244) or AA 521-527 of SEQ ID NO: 240, and minor but detectable reduction at VRDPQTL (SEQ ID NO: 245) or AA 575-583 of SEQ ID NO: 240.

FIG. 8A and FIG. 8B: FIG. 8A. Overlay of the crystal structure of VHH 7A9 (dark grey) bound to the RBD (light grey) with the structure of the Spike protein in the closed state (PDB: 7DF3) showing that the VHH would clash with the NTD of the neighboring protomer. FIG. 8B. Overlay of the crystal structure of VHH 7A9 (dark grey) bound to the RBD (light grey) with the structure of the Spike protein in the open state (PDB: 6XKL) showing that the VHH would clash with the NTD of the neighboring protomer.

FIG. 9A and FIG. 9B: Analytical Ultracentrifugation Absorbance Scans. Raw data A280 absorbance scans from analytical ultracentrifugation experiments. Scans #1-50 (out of 300 total scans) are displayed for each sample. Comparison of panels 3 and 4 shows a dramatic change in sedimentation rate of the spike upon binding to 7A9 VHH, indicating a disruption of the spike trimer upon binding. Comparison of panels 3 and 5 shows a less pronounced change in sedimentation rate of the spike upon binding to 1E4, indicating binding to the trimer without disruption.

### DETAILED DESCRIPTION

### Definitions

Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, the term “spike-protein binder” refers to an antibody, an antibody fragment, a heavy-chain antibody, a heavy-chain antibody fragment (e.g., VHH), or a single domain antibody (also referred to as “sdAb”) that binds to a coronavirus spike protein. A spike-protein binder may be part of a larger molecule such as a multivalent, bispecific, or multispecific binder that includes one or more spike-protein binders and may include one or more binders to a target other than the spike-protein, and may independently comprise another functional element, such as, for example, a half-life extender (HLE), an Fc domain of an immunoglobulin, a targeting unit and/or a molecule such a polyethylene glycol (PEG).

A polypeptide “binds to” a coronavirus spike protein if it has a dissociation constant for binding to the coronavirus spike protein as measured by SPR that is numerically lower than 400 nM. Polypeptides include single-domain antibodies, fragments of single-domain antibodies, as well as constructs that have additional residues over those of single-domain antibodies. When it is stated that a polypeptide or a single-domain antibody binds to more than one protein, that does not require such binding to be simultaneous: the binding can be separate (e.g., the polypeptide or the single-domain antibody binds to protein X when it is contacted with protein X, and the polypeptide or the single-domain antibody binds to protein Y when it is contacted with protein Y).

As used herein, “antibody” refers to an entire immunoglobulin, including recombinantly produced forms and includes any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized antibodies, fully human antibodies, biparatopic antibodies, and chimeric antibodies. “Parental antibodies” are antibodies obtained by exposure of an immune system to an antigen prior to modification of the antibodies for an intended use, such as humanization of a non-human antibody for use as a human therapeutic antibody.

The term “antibody” refers, in one embodiment, to a conventional antibody, which is a protein tetramer comprising two heavy chains (HCs) and two light chains (LCs) inter-connected by disulfide bonds, or an antigen binding portion thereof. In such an embodiment, each heavy chain is comprised of a heavy chain variable region or domain (abbreviated herein as VH) and a heavy chain constant region or domain. In certain naturally occurring IgG, IgD and IgA antibodies, the heavy chain constant region is comprised of three domains, CH1, CH2, and CH3. In certain naturally occurring antibodies, each light chain is comprised of a light chain variable region or domain (abbreviated herein as VL) and a light chain constant region or domain. The light chain constant region is comprised of one domain, CL. The human VH includes six family members: VH1, VH2, VH3, VH4, VH5, and VH6 and the human VL family includes 16 family members: Vκ1, Vκ2, Vκ3, Vκ4, Vκ5, Vκ6, Vλ1, V22, Vλ3, Vλ4, Vλ5, Vλ6, Vλ7, Vλ8, Vλ9, and V210. Each of these family members can be further divided into particular subtypes.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The CDRs form a binding domain that interacts with an antigen.

The constant domains or regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63:78-85 (1969), and is shown for the IgG1, IgG2, IgG3, and IgG4 constant domains in Béranger, et al., Id.

The variable domains or regions of the heavy and light chains contain a binding domain comprising the CDRs that interacts with an antigen. A number of methods are available in the art for defining or predicting the CDR amino acid sequences of antibody variable domains (see Dondelinger et al., Frontiers in Immunol. 9: Article 2278 (2018)). The common numbering schemes include the following:
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196:901-917 (1987); A1-Lazikani et al., J. Mol. Biol. 273:927-948 (1997));
AbM numbering scheme is a compromise between the two used by Oxford Molecular's AbM antibody modelling software (see Karu et al., ILAR Journal 37:132-141 (1995);
Contact numbering scheme is based on an analysis of the available complex crystal structures (See World Wide Web at bioinf.org. uk: Prof. Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509 (1997); Lefranc et al., Dev Comp Immunol. 27:55-77 (2003)).

While there are several different methods for determining the amino acid sequences of the CDRs, the numbering of the entire variable region typically follows the Kabat numbering scheme with the particular CDR numbering scheme imposed thereupon.

The following general rules disclosed in World Wide Web at bioinf.org.uk: Prof. Andrew C. R. Martin's Group and reproduced in Table A below may be used to define or predict the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE A

| Loop | Kabat | AbM | Chothia¹ | Contact² | IMGT |
|---|---|---|---|---|---|
| L1 | L24--L34 | L24--L34 | L26--L32 | L30--L36 | L27--L32 |
| L2 | L50--L56 | L50--L56 | L50--L52 | L46--L55 | L50--L51 |
| L3 | L89--L97 | L89--L97 | L91--L96 | L89--L96 | L89--L97 |
| H1 | H31--H35B | H26--H35B | H26--H32.34 | H30--H35B | H26--H35B |
| | (Kabat | | | | |
| | Numbering)³ | | | | |
| H1 | H31--H35 | H26--H35 | H26--H32 | H30--H35 | H26--H33 |
| | (Chothia | | | | |
| | Numbering) | | | | |
| H2 | H50--H65 | H50--H58 | H52--H56 | H47--H58 | H51--H56 |
| H3 | H95--H102 | H95--H102 | H96--H101 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296:833-849 (2000); Klimka et al., British J. Cancer 83:252-260 (2000); Rader et al., Proc. Natl. Acad. Sci. USA 95:8910⁻⁸⁹¹⁵ (1998); Xu et al., Immunity 13:37-45 (2000)).

A conventional antibody tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)).

The heavy chain of a conventional antibody may or may not contain a terminal lysine (K), or a terminal glycine and lysine (GK).

As used herein, “antigen binding fragment” or “antigen binding portion” refers to fragments of antibodies or of other spike-protein binders, e.g., antibody fragments that retain the ability to bind specifically to the antigen bound by the full-length antibody, such as fragments that retain one or more CDR regions. Examples of antibody binding fragments include, but are not limited to, Fab, Fab′, F(ab′)2, and Fv fragments; diabodies; single-chain antibody molecules, e.g., sc-Fv; immunoglobulin single variable domain molecules, and multispecific antibodies formed from antibody fragments.

As used herein, the term “immunoglobulin single variable domain” (also referred to as “ISV” or ISVD″) or “single domain antibody (also referred to as “sdAb”) are terms that are used to refer to immunoglobulin variable domains (which may be heavy chain or light chain domains, including VH, VHH, or VL domains) that can form a functional antigen-binding site without interaction with another variable domain (e.g., without a VH/VL interaction as is required between the VH and VL domains of a conventional four-chain monoclonal antibody). The term “VH” refers to a heavy chain variable domain of a conventional antibody and the term “VHH” refers to the heavy chain variable domain of a non-conventional heavy chain antibody.

Examples of ISVDs include for example, VHHs, humanized VHHs, and/or a camelized VHs such as camelized human VHs), IgNAR domains, single domain antibodies such as domain antibodies (dAbs), which are VH domains or are derived from a VH domain or are VL domains or are derived from a VL domain. ISVDs that are based on and/or derived from heavy chain variable domains (such as VH or VHH domains) are generally preferred. Most preferably, an ISVD will be a VHH, a humanized VHH, or a camelized VH (such as a camelized human VH) or generally a sequence optimized VHH (e.g., optimized for chemical stability and/or solubility, improved overlap with known human framework regions and improved expression).

The term “VHH” as used herein indicates that the heavy chain variable domain is obtained from or originated or derived from a heavy chain antibody. Heavy chain antibodies are functional antibodies that have two heavy chains and no light chains. Heavy chain antibodies exist in and are obtainable from Camelids (e.g., camels and alpacas), members of the biological family Camelidae. VHH antibodies were originally described as the antigen binding immunoglobulin (variable) domain of “heavy chain antibodies” (i.e., of “antibodies devoid of light chains”; Hamers-Casterman et al., Nature 363:446-448 (1993)). The term “VHH domain” has been chosen in order to distinguish these variable domains from the heavy chain variable domains that are present in conventional four-chain antibodies (which are referred to herein as “VH domains” or “VH”) and from the light chain variable domains that are present in conventional four-chain antibodies (which are referred to herein as “VL domains” or “VL”). For a further description of VHHs, reference is made to the review article by Muyldermans (Reviews in Molec. Biotechnol. 74:277-302, (2001), as well as to the following patent applications, which are mentioned as general background art: WO9404678, WO9504079 and WO9634103 of the Vrije Universiteit Brussel; WO9425591, WO9937681, WO0040968, WO0043507, WO0065057, WO0140310, WO0144301, EP1134231 and WO0248193 of Unilever; WO9749805, WO0121817, WO03035694, WO03054016 and WO03055527 of the Vlaams Instituut voor Biotechnologie (VI B); WO03050531 of Algonomics N.V. and Ablynx N.V.; WO0190190 by the National Research Council of Canada; WO03025020 (=EP 1433793) by the Institute of Antibodies; as well as WO2004041867, WO2004041862, WO2004041865, WO2004041863, WO2004062551, WO2005044858, WO200640153, WO2006079372, WO2006122786, WO06122787, WO2006122825, WO2008101985, WO2008142164, and WO2015173325).

The term “Nanobody® molecule” (Ablynx N.V., Ghent BE) is generally as defined in WO 2008/020079 or WO 2009/138519, and thus, in a specific aspect denotes a VHH, a humanized VHH, or a camelized VH (such as a camelized human VH) or generally a sequence optimized VHH (such as, e.g., optimized for chemical stability and/or solubility, maximum overlap with known human framework regions and maximum expression).

For a general description of multivalent and multispecific polypeptides containing one or more ISVDs and their preparation, reference is also made to Conrath et al., J. Biol. Chem., Vol. 276, 10:7346-7350 (2001); Muyldermans, Reviews in Molecular Biotechnology 74:277-302 (2001); as well as to, for example, WO 1996/34103, WO 1999/23221, WO 2004/041862, WO 2006/122786, WO 2008/020079, WO 2008/142164 and WO 2009/068627.

As used herein, a “Fab fragment” is comprised of one light chain and the CH1 and variable regions of one heavy chain. The heavy chain of a Fab molecule cannot form a disulfide bond with another heavy chain molecule. A “Fab fragment” can be the product of papain cleavage of an antibody.

As used herein, a “Fab′ fragment” contains one light chain and a portion or fragment of one heavy chain that contains the VH domain and the CH1 domain and also the region between the CH1 and CH2 domains, such that an interchain disulfide bond can be formed between the two heavy chains of two Fab′ fragments to form a F(ab′)2molecule.

As used herein, a “F(ab′)2 fragment” contains two light chains and two heavy chains containing the VH domain and a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains. A “F(ab′)2 fragment” can be the product of pepsin cleavage of an antibody.

As used herein, an “Fv fragment” comprises the variable regions from both the heavy and light chains but lacks the constant regions.

These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

As used herein, an “Fc domain” or “Fc region” each refer to the fragment crystallizable region of an antibody. The Fc domain comprises two heavy chain fragments comprising the CH2 and CH3 domains of an antibody. The two heavy chain fragments are held together by two or more disulfide bonds and by hydrophobic interactions of the CH3 domains. The Fc domain may be fused at the N-terminus or the C-terminus to a heterologous protein.

As used herein, “isolated” antibodies or antigen-binding fragments thereof (e.g., spike-protein binders) are at least partially free of other biological molecules from the cells or cell cultures in which they are produced. Such biological molecules include nucleic acids, proteins, lipids, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, a “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies; it is not to be construed as requiring production of the antibody by any particular method. For example, certain spike-protein binders, their fragments, or their further constructs to be used in accordance with certain embodiments of the invention may be made by the hybridoma method first described by Kohler et al. (1975) Nature 256:495, or they may be made by recombinant DNA methods (see, e.g., U.S. Pat. No. 4,816,567). Certain spike-protein binders, their fragments, or their further constructs may also be isolated from phage antibody libraries using the techniques described in Clackson et al. (1991) Nature 352:624-628 and Marks et al. (1991) J. Mol. Biol. 222:581-597. See also Presta (2005) J. Allergy Clin. Immunol. 116:731.

As used herein, a “humanized ISVD” or “humanized antibody” refers to forms of spike-protein binders that contain sequences from both human and non-human (e.g., llama, murine, rat) antibodies. In general, the humanized spike-protein binders will comprise all of at least one variable domain in which the hypervariable loops correspond to those of a non-human immunoglobulin, and all or substantially all of the framework (FR) regions are those of a human immunoglobulin sequence. The humanized spike-protein binder may optionally comprise at least a portion of a human immunoglobulin constant region (Fc).

“Humanization” (also called reshaping or CDR-grafting) is now a well-established technique for reducing the immunogenicity of monoclonal antibodies (mAbs) from xenogeneic sources (commonly rodent or camelids) and for improving the effector functions (ADCC, complement activation, C1q binding). The engineered mAb is engineered using the techniques of molecular biology, however simple CDR-grafting of the rodent complementarity-determining regions (CDRs) into human frameworks often results in loss of binding affinity and/or specificity of the original mAb. In order to humanize an antibody, the design of the humanized antibody includes variations such as conservative amino acid substitutions in residues of the CDRs, and back substitution of residues from the rodent mAb into the human framework regions (backmutations). The positions can be discerned or identified by sequence comparison for structural analysis or by analysis of a homology model of the variable regions' 3D structure. The process of affinity maturation has most recently used phage libraries to vary the amino acids at chosen positions. Similarly, many approaches have been used to choose the most appropriate human frameworks in which to graft the rodent CDRs. As the datasets of known parameters for antibody structures increase, so do the sophistication and refinement of these techniques. Consensus or germline sequences from a single antibody or fragments of the framework sequences within each light or heavy chain variable region from several different human mAbs can be used. Another approach to humanization is to modify only surface residues of the rodent sequence with the most common residues found in human mAbs and has been termed “resurfacing” or “veneering.” Known human Ig sequences are disclosed, e.g., in World Wide Web at ncbi.nlm.nih.gov/entrez/query.fcgi; ncbi.nih.gov/igblast; atcc.org/phage/hdb.html; kabatdatabase.com/top.html; antibodyresource.com/onlinecomp.html; appliedbiosystems.com; biodesign.com; antibody.bath.ac.uk; unizh.ch; cryst.bbk.ac.uk/.about.ubcg07s; Kabat et al., Sequences of Proteins of Immunological Interest, U.S. Dept. Health (1983), each entirely incorporated herein by reference. Often, the human or humanized antibody is substantially non-immunogenic in humans.

As used herein, a “non-human amino acid sequence” with respect to antibodies or immunoglobulins refers to an amino acid sequence that is characteristic of the amino acid sequence of a non-human mammal. The term does not include amino acid sequences of antibodies or immunoglobulins obtained from a fully human antibody library where diversity in the library is generated in silico (See e.g., U.S. Pat. No. 8,877,688 or 8,691,730).

As used herein, “effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

As used herein, “conservatively modified variants” or “conservative substitution” refers to substitutions of amino acids with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table B below.

TABLE B

| Original | Conservative | Original | Conservative |
|---|---|---|---|
| residue | substitution | residue | substitution |
| Ala (A) | Gly; Ser | Leu (L) | Ile; Val |
| Arg (R) | Lys; His | Lys (K) | Arg; His |
| Asn (N) | Gln; His | Met (M) | Leu; Ile; Tyr |
| Asp (D) | Glu; Asn | Phe (F) | Tyr; Met; Leu |
| Cys (C) | Ser; Ala | Pro (P) | Ala |
| Gln (Q) | Asn | Ser (S) | Thr |
| Glu (E) | Asp; Gln | Thr (T) | Ser |
| Gly (G) | Ala | Trp (W) | Tyr; Phe |
| His (H) | Asn; Gln | Tyr (Y) | Trp; Phe |
| Ile (I) | Leu; Val | Val (V) | Ile; Leu |

As used herein, “mutations” include substitutions (e.g., conservative substitutions). Mutations also include deletions and insertions (e.g., appearing as gaps in a sequence alignment).

As used herein, the term “epitope” or “antigenic determinant” refers to a site on an antigen (e.g., spike-protein) to which a binder specifically binds. Epitopes within protein antigens can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. A contiguous linear epitope comprises a peptide domain on an antigen comprising at least 3, 4, 5, 6, 7, 8, 9, 10, 11, 12, 13, 14 or 15 amino acids. A noncontiguous conformational epitope comprises one or more peptide domains or regions on antigen bound by a binder interspersed by one or more amino acids or peptide domains not bound by the binder, each domain independently comprises at least 3, 4, 5, 6, 7, 8, 9, 10, 11, 12, 13, 14 or 15 amino acids. Methods for determining what epitopes are bound by a given binder (i.e., epitope mapping) are well known in the art and include, for example, immunoblotting and immunoprecipitation assays, wherein overlapping or contiguous peptides (e.g., from spike-protein) are tested for reactivity with a given binder. Methods of determining spatial conformation of epitopes include techniques in the art and those described herein, for example, x-ray crystallography, 2-dimensional nuclear magnetic resonance, and HDX-MS (see, e.g., Epitope Mapping Protocols in Methods in Molecular Biology, Vol. 66, G. E. Morris, Ed. (1996)).

The term “epitope mapping” refers to the process of identification of the molecular determinants on the antigen involved in antibody-antigen recognition.

The term “binds to the same epitope” with reference to two or more binders means that the binders bind to the same segment of amino acid residues on a target, as determined by a given method. Techniques for determining whether a particular binder binds to the “same epitope” as the spike-protein binders described herein include, for example, epitope mapping methods, such as, x-ray analyses of crystals of spike-protein: spike-protein-binder complexes, which provides atomic resolution of the epitope, and hydrogen/deuterium exchange mass spectrometry (HDX-MS). Other methods that monitor the binding of the antibody to antigen fragments (e.g., proteolytic fragments) or to mutated variations of the antigen where loss of binding due to a modification of an amino acid residue within the antigen sequence is often considered an indication of an epitope component (e.g., alanine scanning mutagenesis—Cunningham & Wells (1985) Science 244:1081). In addition, computational combinatorial methods for epitope mapping can also be used. These methods rely on the ability of the binder of interest to affinity isolate specific short peptides from combinatorial phage display peptide libraries.

Binders that “compete with a binder of the invention for binding to a target antigen” refer to binders that inhibit (partially or completely) the binding of the spike-protein binder of the invention to spike-protein. Whether two binders compete with each other for binding to the target antigen, i.e., whether and to what extent one binder inhibits the binding of the other binder to the target antigen, may be determined using known competition experiments. In certain embodiments, a binder competes with, and inhibits binding of a binder of the invention to the target antigen by at least 10%, 20%, 30%, 40%, 50%, 60%, 70%, 80%, 90% or 100%. The level of inhibition or competition may be different depending on which binder is the “blocking binder” (i.e., the unlabeled binder that is incubated first with the target antigen). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing spike-protein binders bind to the same epitope (e.g., one of the nine described epitopes) as defined herein.

Other competitive binding assays include solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, “specifically binds” refers, with respect to a target antigen, to the preferential association of a binder, in whole or part, with the target antigen and not to other molecules, particularly molecules found in human blood or serum. Binders as shown herein typically bind specifically to the target antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, a binder that “specifically binds” or “binds specifically” to a target antigen refers to a binder that binds to the target antigen with high affinity, which means having a K_{D} of 10⁻⁷ M or less, in particular embodiments a K_{D} of 10⁻⁸ M or less, or 5×10⁻⁹ M or less, or between 10⁻⁸ M and 10⁻¹¹ M or less, as determined in a cell ELISA or Surface Plasmon Resonance assay (SPR; e.g., Biacore™) using 10 μg/mL antibody.

As used herein, a K_{D} is “numerically lower” in its conventional sense: when the affinity is stronger. For example, a K_{D} of 1 pM is numerically lower than a K_{D} of 1 nM.

As used herein, an antigen is “substantially identical” to a given antigen if it exhibits a high degree of amino acid sequence identity to the given antigen, for example, if it exhibits at least 80%, at least 90%, at least 95%, at least 97%, or at least 99% or greater amino acid sequence identity to the amino acid sequence of the given antigen.

As used herein, “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

“Treat” or “treatment” means to alleviate one or more disease symptoms in the treated subject or population, whether by inducing the regression of or inhibiting, delaying or slowing the progression of such symptom(s) by any clinically measurable degree. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom. Typically, treatment is achieved via administration of a therapeutic agent, such as a composition containing any of the single-domain antibodies, antibodies, or antigen binding fragments of the invention, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject.

As used herein, the term “subject” (alternatively referred to as “patient” or “individual” herein) refers to an organism, typically a mammal (e.g., rat, mouse, dog, cat, rabbit, human, in some embodiments including prenatal human forms) capable of being treated with a polypeptide, single-domain antibody, composition, or method of the disclosure, most preferably a human. In some embodiments, the subject is an adult patient. In other embodiments, the subject is a pediatric patient. A subject “in need of treatment” means that the subject has been identified as having a need for the particular composition or treatment and may benefit from treatment with the methods disclosed herein, e.g. a subject suffering from or at risk of developing a disease or disorder, e.g. COVID-19. In some embodiments, a subject displays one or more symptoms or characteristics of a disease, disorder or condition that any polypeptide, single domain antibody, or composition of the disclosure is meant to treat (e.g. SARS, MERS or COVID-19). In some embodiments, a subject does not display any symptom or characteristic of such disease, disorder, or condition, but has tested positive for infection of a coronavirus, has been diagnosed with a coronavirus infection, or is at risk for developing a coronavirus infection.

As used herein, “therapeutically effective amount” refers to a quantity of a specific substance sufficient to achieve a desired effect in an individual being treated. For instance, this may be the amount necessary to inhibit or reduce the effects or severity of SARS, MERS, or COVID-19 or any symptom thereof in an individual, e.g. decrease shortness of breath or reduce difficulty breathing.

As used herein, the term “effector-silent” refers to an antibody, antibody fragment, HC constant domain, or Fc domain thereof that displays (i) no measurable binding to one or more Fc receptors (FcRs) as may be measured in a surface plasmon resonance (SPR) assay (e.g., Biacore™ assay) wherein an association constant in the micromolar range indicates no measurable binding or (ii) measurable binding to one or more FcRs as may be measured in SPR assay that is reduced compared to the binding that is typical for an antibody, antibody fragment, HC constant domain, or Fc domain thereof the same isotype. In particular embodiments, the antibody, antibody fragment, HC constant domain, or Fc domain thereof may comprise one or more mutations in the HC constant domain and the Fc domain in particular such that the mutated an antibody, antibody fragment, HC constant domain or Fc domain thereof has reduced or no measurable binding to FcγRIIIa, FcγRIIa, and FcγRI compared to a wild-type antibody of the same isotype as the mutated antibody. In particular embodiments, the affinity or association constant of an effector-silent antibody, antibody fragment, HC constant domain or Fc domain thereof to one or more of FcγRIIIa, FcγRIIa, and FcγRI is reduced by at least 1000-fold compared to the affinity of the wild-type isotype; reduced by at least 100-fold to 1000-fold compared to the affinity of the wild-type isotype reduced by at least 50-fold to 100-fold compared to the affinity of the wild-type isotype; or at least 10-fold to 50-fold compared to the affinity of the wild-type isotype. In particular embodiments, the effector-silent antibody, antibody fragment, HC constant domain, or Fc domain thereof has no detectable or measurable binding to one or more of the FcγRIIIa, FcγRIIa, and FcγRI as compared to binding by the wild-type isotype. In general, an effector-silent antibody, antibody fragment, HC constant domain, or Fc domain thereof will lack measurable antibody-dependent cell-mediated cytotoxicity (ADCC) activity. An ISVD fused or linked to an effector-silent HC constant domain or Fc domain thereof displays no detectable or measurable binding to one or more of FcγRIIIa, FcγRIIa, or FcγRI. SPR assays measure binding of an effector-silent antibody, antibody fragment, HC constant domain or Fc domain thereof, against human FcRs.

The term “congruent with each other in modulo 30” has its customary mathematical meaning. As a simple illustration of this concept in the context of hours in a day, one may think of “1” and “13” as being congruent with each other in modulo 12, since 13 of a 24-hour system is the same as the 1 μm of a 12-hour system. More precisely, in this example “1” and “13” are congruent with each other since when divided by 12 (of “modulo 12”), the remainder in each case is the same (“1”). Another illustration is provided in Table D, in which one can see that the SEQ ID NOs for the CDR1, CDR2, and CDR3 of a specified construct in a row are congruent with each other in modulo 30. For example, the following SEQ ID NO's for CDR1, CDR2, and CDR3 are congruent with each other in modulo 30:

TABLE C

| | | | | |
|---|---|---|---|---|
| 31, 61, and 91 | 32, 62, and 92 | 33, 63, and 93 | 34, 64, and 94 | 35, 65, and 95 |
| 36, 66, and 96 | 37, 67, and 97 | 38, 68, and 98 | 39, 69, and 99 | 40, 70 and 100 |
| 41, 71, and101 | 42, 72, and 102 | 43, 73, and 103 | 44, 74, and 104 | 45, 75, and 105 |
| 46, 76, and 106 | 47, 77, and 107 | 48, 78, and 108 | 49, 79, and 109 | 50, 80, and 110 |
| 51, 81, and 111 | 52, 82, and 112 | 53, 83, and 113 | 54, 84, and 114 | |

Table D also illustrates that the SEQ ID NOs for the CDR1, CDR2, and CDR3 of constructs from different rows are not congruent with each other in modulo 30, since the number of constructs in that table, 24, is less than 30.

Two coronaviruses are “different coronaviruses” if they are of different species (e.g., SARS-CoV, SARS-CoV2, and MERS-CoV) or of different variants of the same species (e.g., SARS-CoV2 variants B.1.1.7, B.1.351, P.1, B.1.617.2, and B.1.1.529).

### Coronavirus Spike Proteins

CoV particles consist of a cell-derived lipid membrane containing structural proteins spike(S), membrane (M), envelope (E), and nucleocapsid (N) (Fields Virology Emerging Viruses Vol. 1 2021 pp. 416-417). The virion also contains a large (25-32 kb) non-segmented positive-sense single-strand viral RNA genome that, similar to cellular mRNAs, is 5′-capped, contains 5′ and 3′ untranslated regions (UTRs) and a 3′ polyadenylated tail. All CoV viral genomes contain six basic common genes: two long open reading frames (1a and 1b) that encode two polypeptides that constitute the non-structural proteins (nsps) that form the multiprotein replicase-transcription complex (RTC) and four open reading frames for the structural proteins S, M, E and N that make up the virion. Depending on the CoV, one to eight additional genes, called accessory genes, can be encoded in the genome. The genomic organization amongst all CoVs is conserved and invariant across different genera such that the gene sequence is always 1a, 1b, S, M, E and N.

CoV replication is initiated through binding of the S protein to a specific cell surface receptor. SARS-CoV and SARS-CoV-2, for example, engage the angiotensin converting enzyme 2 (ACE-2) on cells of the upper respiratory tract (Lu R, Zhao X, Li J, et al. 2020. Lancet; 395 (10224): 565-574). Viral attachment leads to either viral endocytosis followed by fusion of the viral and endosome membranes, or direct fusion of the viral and cellular plasma members at the cell surface, to release virions into the cytoplasm. After entry, the viral genomic RNA is uncoated and serves as a template for cap-dependent translation of Orf 1a and Orf 1b to produce the viral polypeptides pp1a and pp1ab (Fung S, Liu D, 2019. Annu. Rev. Microbiol. 73:529-57).

Cleavage of the viral polypeptides to yield the individual replisome proteins is carried out by the viral papain-like protease (PLPro or nsp3) and 3CL main protease (Mpro or nsp5). The nsps form double-membraned vesicles and assemble to form RTCs responsible for genome replication, sub-genomic RNA (sgRNA) synthesis and transcription of the sgRNAs. The sgRNA serve as templates from which the mRNAs encoding for the structural and accessory proteins are translated. Assembly of new viral particles occurs in the endoplasmic reticulum-golgi intermediate complex and mature particles are released through secretory vesicles.

Vaccines for prevention of COVID-19 have been developed using the S protein of SARS-CoV-2 as an antigen to elicit a protective immune response (Kryikidis et. al. npj Vaccines 28 (2021) 6:28). Vaccines based on mRNA/lipid nanoparticle and replication-defective adenoviruses vectored platforms have both been demonstrated to be highly effective for prevention of serious illness. However, there is limited data on the effectiveness of these vaccines for transmission of SARS-CoV-2. A liability of using the S protein for vaccine development is that the amino acid sequence is highly variable, enabling the SARS-CoV-2 to adapt to immune pressure (Chen R E et al. 2021 Nature Medicine 27 (4): 717-726). Multiple independent spike mutations have been detected, even in the absence of vaccine selective pressure, and some variants will likely lead to reduced efficacy in vaccine clinical trials conducted where those variants are circulating.

The single-domain antibodies and other polypeptides of the invention, including polypeptides with multiple spike-protein binders, are able to bind to two or more spike proteins from different coronaviruses, and thus do not have the described limitations of the vaccines.

### Spike-Protein Binders

Among the provided embodiments are 24 single-domain antibodies, which are identified via a sample name in the first column of Table D, and via a VHH name in the second column of Table D. Subsequent columns of Table D also provide SEQ ID NOs of the variable domains as well as of certain CDR1, CDR2, and CDR3s of these embodiments. A sample ID for the clones would include “BCC.AS008” before the clone name (e.g., the Sample ID for Clone “10B8” is “BCC.AS008.10B8”).

TABLE D

| | | VHH | CDR1 | CDR2 | CDR3 |
|---|---|---|---|---|---|
| | VHH | SEQ ID | SEQ ID | SEQ ID | SEQ ID |
| Clone Name: | Name: | NO: | NO: | NO: | NO: |
| 10B8 | VHH1 | 1 | 31 | 61 | 91 |
| 1E4 | VHH2 | 2 | 32 | 62 | 92 |
| 16H7 | VHH3 | 3 | 33 | 63 | 93 |
| 5G11 | VHH4 | 4 | 34 | 64 | 94 |
| 13C8 | VHH5 | 5 | 35 | 65 | 95 |
| 3D4 | VHH6 | 6 | 36 | 66 | 96 |
| 15H1 | VHH7 | 7 | 37 | 67 | 97 |
| S3_44 | VHH8 | 8 | 38 | 68 | 98 |
| 11F5 | VHH9 | 9 | 39 | 69 | 99 |
| 1A11 | VHH10 | 10 | 40 | 70 | 100 |
| 14F3 | VHH11 | 11 | 41 | 71 | 101 |
| 15H7 | VHH12 | 12 | 42 | 72 | 102 |
| 11F8 | VHH13 | 13 | 43 | 73 | 103 |
| 19C10 | VHH14 | 14 | 44 | 74 | 104 |
| S3_29 | VHH15 | 15 | 45 | 75 | 105 |
| 8E9 | VHH16 | 16 | 46 | 76 | 106 |
| 7A9 | VHH17 | 17 | 47 | 77 | 107 |
| 5D7 | VHH18 | 18 | 48 | 78 | 108 |
| 20D11 | VHH19 | 19 | 49 | 79 | 109 |
| 9B2 | VHH20 | 20 | 50 | 80 | 110 |
| 19B8 | VHH21 | 21 | 51 | 81 | 111 |
| 5A6 | VHH22 | 22 | 52 | 82 | 112 |
| 19E10 | VHH23 | 23 | 53 | 83 | 113 |
| 6A1 | VHH24 | 24 | 54 | 84 | 114 |

Even though the sequences identified in the third column of Table D would form single-domain antibodies, one may alternatively start with one, two, or three of the CDRs without the rest of the VHHs, and then construct using suitable approaches a spike-protein binder that is not a single-domain antibody. For example, one may even generate specific antibodies based on CDR3 of the heavy chain (e.g., Beiboer et al., J. Mol. Biol. 296:833-849 (2000); Klimka et al., British J. Cancer 83:252-260 (2000); Rader et al., Proc. Natl. Acad. Sci. USA 95:8910-8915 (1998); Xu et al., Immunity 13:37-45 (2000)).

In some embodiments, the spike-protein binders (e.g., single-domain antibodies, conventional antibodies) comprise the CDR1, CDR2, and CDR3 provided in any row of Table D with one to three total (i.e., 1, 2, or 3) residue mutations among themselves. When the total residue mutations is three, the number of mutations in CDR1, CDR2, and CDR3, respectively and with respect to the SEQ ID NOs provided in Table D, can be 3, 0, 0; 2, 1, 0; 2, 0, 1; 1, 2, 0; 1, 1, 1; 1, 0, 2; 0, 3, 0; 0, 2, 1; 0, 1, 2; or 0, 0, 3.

In some embodiments, the spike-protein binders (e.g., single-domain antibodies, conventional antibodies) comprise the CDR1, CDR2, and CDR3 provided in any row of Table D with four to six (i.e., 4, 5, or 6) total residue mutations among themselves.

In some embodiments, the spike-protein binders comprise the FR1, FR2, FR3, and FR4 having the sequence of any one of SEQ ID NOs 121 to 144, 151 to 174, 181 to 204, and 211 to 234 respectively with one to nine (i.e., 1, 2, 3, 4, 5, 6, 7, 8, or 9) total residue mutations among themselves (e.g., 3 in FR1, 2 in FR2, 3 in FR3, and 1 in FR4 when there are nine total residue mutations among these). In some embodiments, the spike-protein binders comprise the FR1, FR2, FR3, and FR4 respectively having the sequence of any one of SEQ ID NOs 121 to 144, 151 to 174, 181 to 204, and 211 to 234 with ten to sixteen (i.e., 10, 11, 12, 13, 14, 15, 16) total residue mutations among themselves. In some embodiments, these framework regions have the sequences that correspond to the parts of VHHs provided in Table D that are outside of the CDRs. As an example, for VHH1, the FR1, FR2, FR3, and FR4 would have the sequences with SEQ ID NOs 121, 151, 181, and 211, and for VHH24, the FR1, FR2, FR3, and FR4 would have the sequences with SEQ ID NOs 144, 174, 204, and 234. In addition, in some embodiments, the FR1, FR2, FR3, and FR4 have a sequence (e.g., SEQ ID NOs 146, 176, 206, and 236, respectively) that has residues with the highest occurrences in their positions in the set of Table D, or a sequence (e.g., SEQ ID NOs 145, 175, 205, and 235, respectively) that is a consensus sequence that allows for redundant substitutions. Some of these embodiments are represented in Table E.

TABLE E

| | FR1 | FR2 | FR3 | FR4 |
|---|---|---|---|---|
| | SEQ ID | SEQ ID | SEQ ID | SEQ ID |
| VHH Name: | NO: | NO: | NO: | NO: |
| VHH1 | 121 | 151 | 181 | 211 |
| VHH2 | 122 | 152 | 182 | 212 |
| VHH3 | 123 | 153 | 183 | 213 |
| VHH4 | 124 | 154 | 184 | 214 |
| VHH5 | 125 | 155 | 185 | 215 |
| VHH6 | 126 | 156 | 186 | 216 |
| VHH7 | 127 | 157 | 187 | 217 |
| VHH8 | 128 | 158 | 188 | 218 |
| VHH9 | 129 | 159 | 189 | 219 |
| VHH10 | 130 | 160 | 190 | 220 |
| VHH11 | 131 | 161 | 191 | 221 |
| VHH12 | 132 | 162 | 192 | 222 |
| VHH13 | 133 | 163 | 193 | 223 |
| VHH14 | 134 | 164 | 194 | 224 |
| VHH15 | 135 | 165 | 195 | 225 |
| VHH16 | 136 | 166 | 196 | 226 |
| VHH17 | 137 | 167 | 197 | 227 |
| VHH18 | 138 | 168 | 198 | 228 |
| VHH19 | 139 | 169 | 199 | 229 |
| VHH20 | 140 | 170 | 200 | 230 |
| VHH21 | 141 | 171 | 201 | 231 |
| VHH22 | 142 | 172 | 202 | 232 |
| VHH23 | 143 | 173 | 203 | 233 |
| VHH24 | 144 | 174 | 204 | 234 |
| Consensus | 145 | 175 | 205 | 235 |
| w/alternatives | | | | |
| Consensus w/singles | 146 | 176 | 206 | 236 |

The mutations with respect to any of the disclosed sequences can be deletions (e.g., represented as a gap in the mutated sequence when aligned to the reference sequence), insertions (e.g., represented as a gap in the reference sequence when aligned to the mutated sequence), or substitutions. The substitutions are, in some embodiments, conservative substitutions (e.g., those shown in Table B).

Additional embodiments can be created by varying the provided framework regions. In some embodiments, the ISVD framework comprises one or more substitutions to minimize binding to pre-existing antibodies. Pre-existing antibodies are antibodies existing in the body of a patient prior to receipt of an ISVD and are immunoglobulins mainly of the IgG class that are present in varying degrees in up to 50% of the human population and that bind to critical residues clustered at the C-terminal region of ISVDs. The ISVDs of the invention are based, in part, in llama antibodies whose C-terminal constant domains have been removed; thus, exposing the neo-epitopes in the C-terminus of the resulting VHH to pre-existing antibody binding. It has been discovered that the combination of mutations of residues 11 and 89 (e.g., L11V and I89L or V89L) led to a surprising lack of pre-existing antibody binding. Mutations in residue 112 have also been shown to remarkably reduce pre-existing antibody binding. Buyse & Boutton (WO2015/173325) included data showing that the combination of an L11V and V89L mutation provided a remarkable improvement in reducing pre-existing antibody binding compared to an L11V mutation alone or a V89L mutation alone. For example, Table H of Buyse & Boutton on page 97 showed comparative data for an ISVD with a V89L mutation alone (with or without C-terminal extension) and the same ISVD with a V89L mutation in combination with an L11V mutation (again, with or without a C-terminal extension). Also, although generated in two separate experiments, comparing the L11V/V89L combination to the L11V mutation alone (in the same ISVD) showed that the pre-existing antibody binding reduction that is obtained by the L11V/V89L combination was greater than that for the LIIV mutation alone. Since the llama antibody scaffold structure is known to be very highly conserved, the effect of the mutations at positions 11 and 89 is very likely to exist for any ISVD. Thus, in embodiments herein, the ISVD comprises at least the L11V/V89L substitutions in the framework regions.

In a further embodiment, FR1 comprises at least an L11V substitution and FR3 comprises at least a V89L substitution. In any one of the above embodiments, the FR1 may further comprise a Q1E or a Q1D amino acid substitution.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689.

In particular embodiments, it will be desirable to change certain amino acids containing exposed side-chains to another amino acid residue in order to provide for greater chemical stability of the final antibody, as follows. The deamidation of asparagine may occur on N-G or D-G sequences and result in the creation of an isoaspartic acid residue that introduces a kink into the polypeptide chain and decreases its stability (isoaspartic acid effect). In certain embodiments, the single-domain antibodies, antibodies, or antigen-binding fragments of the present disclosure do not contain asparagine isomerism sites.

For example, an asparagine (Asn) residue may be changed to Gln or Ala to reduce the potential for formation of isoaspartate at any Asn-Gly sequences, particularly within a CDR. A similar problem may occur at a Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734. In one embodiment, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe in order to reduce the possibility that the methionine sulfur would oxidize, which could reduce antigen binding affinity and also contribute to molecular heterogeneity in the final antibody preparation. Id. In one embodiment, the methionine is changed to alanine (Ala). Additionally, in order to prevent or minimize potential scissile Asn-Pro peptide bonds, it may be desirable to alter any Asn-Pro combinations found in a CDR to Gln-Pro, Ala-Pro, or Asn-Ala. Single-domain antibodies, antibodies, or antigen-binding fragments with such substitutions are subsequently screened to ensure that the substitutions do not decrease the affinity or specificity of the single-domain antibodies, antibodies, or antigen-binding fragments for the target, or other desired biological activity to unacceptable levels.

TABLE F
Exemplary stabilizing CDR variants

| CDR Residue | Stabilizing Variant Sequence |
|---|---|
| Asn-Gly | Gln-Gly, Ala-Gly, or Asn-Ala |
| (N-G) | (Q-G), (A-G), or (N-A) |
| Asp-Gly | Glu-Gly, Ala-Gly or Asp-Ala |
| (D-G) | (E-G), (A-G), or (D-A) |
| Met (typically solvent exposed) | Lys, Leu, Ala, or Phe |
| (M) | (K), (L), (A), or (F) |
| Asn | Gln or Ala |
| (N) | (Q) or (A) |
| Asn-Pro | Gln-Pro, Ala-Pro, or Asn-Ala |
| (N-P) | (Q-P), (A-P), or (N-A) |

In some embodiments, the disclosed single-domain antibodies or other polypeptides, in their monovalent forms, bind to at least two spike proteins with an EC50 value that is numerically lower than any of these values in nanomolar: 400, 350, 300, 250, 200, 150, 100, 90, 80, 70, 60, 50, 40, 30, 20, 10, 5, 1, 0.5, 0.4, 0.3, 0.2, 0.1, 0.05, or 0.01 as measured by an ELISA assay.

In some embodiments, the disclosed single-domain antibodies or other polypeptides, in their monovalent forms, bind to at least two spike proteins with a K_{D} value that is numerically lower than any of these values in nanomolar: 400, 350, 300, 250, 200, 150, 100, 90, 80, 70, 60, 50, 40, 30, 20, 10, 5, 1, 0.5, 0.4, 0.3, 0.2, 0.1, 0.05, or 0.01 as measured by SPR.

In some embodiments, the disclosed single-domain antibodies or other polypeptides, in their bivalent forms (e.g., when expressed as a fusion protein with an Fc, so that it forms a dimer), bind to at least two spike proteins with an EC50 value that is numerically lower than any of these values in nanomolar: 10, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, or 0.001 as measured by an ELISA assay.

In some embodiments, the disclosed single-domain antibodies or other polypeptides, in their bivalent forms (e.g., when expressed as a fusion protein with an Fc, so that it forms a dimer), bind to at least two spike proteins with a K_{D} value that is numerically lower than any of these values in nanomolar: 10, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, or 0.001 as measured by SPR.

In some embodiments, the disclosed single-domain antibodies or other polypeptides, when in monovalent or in divalent form, separately inhibit infection of Vero-E6 cells by said at least two different coronaviruses with an IC50 value that is numerically lower than any of these values in nanomolar: 400, 350, 300, 250, 200, 150, 100, 90, 80, 70, 60, 50, 40, 30, 20, 10, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, or 0.001.

Also disclosed are polypeptides that contain multimers of spike-protein binders (e.g., a first spike-protein binder, a second spike-protein binder, and a third spike-protein binder). In some embodiments, the spike-protein binder is a single-domain antibody (e.g., one of those disclosed in Table D). In such embodiments, each spike-protein binder can be independently selected from any of the polypeptides or single-domain antibodies disclosed herein. By such an independent selection, one may have a polypeptide in which all spike-protein binders are the same (e.g., to enhance affinity to multiple copies of the same binding site) or some or all can be different (e.g., to ensure binding to at least one of the different binding sites in case some binding sites are unknown or are mutated). Thus, in some embodiments, the spike-protein binders independently bind to an NTD, an S2, or an RBD of the spike protein (e.g., they all individually bind to RBD; they collectively bind to each of NTD, S2, and RBD; they collectively bind to RBD and S2).

In some embodiments, the polypeptides that comprise multiple spike-protein binders further comprise linkers between one or more (e.g., all) of the spike-protein binders. The linkers can contain 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 residues (e.g., amino acids).

As an example, for a polypeptide with three spike-protein binders, each of the two linkers can contain 14 to 26 residues (e.g., 20 amino acids each). Such a polypeptide can be useful, for example, when all three of the spike-protein binders are single-domain antibodies that bind to an epitope on the RBD.

As another example, for a polypeptide with three spike-protein binders, the first linker can contain 14 to 26 residues (e.g., 20 amino acids) while the other linker contains 40 to 60 residues (e.g., 50 amino acids). Such a polypeptide can be useful, for example, when all three of the spike-protein binders are single-domain antibodies, and the first one binds the RBD, the middle on binds the NTD, and the third one (linked via the longer linker) binds the S2 of a spike protein.

Various such constructs can be generated, for example based on the data provided in the Examples (e.g., Tables 1A, 1B, 2, 3, 4, 5, 7, and 8).

Spike-protein binders of the invention can be fused or linked to one or more other amino acid sequences, chemical entities or moieties by a peptide or non-peptide linker. These other amino acid sequences, chemical entities or moieties can confer one or more desired properties to the resulting spike-protein binders of the invention, for example, to provide the resulting spike-protein binders of the invention with affinity against another therapeutically relevant target such that the resulting polypeptide becomes “bispecific” with respect to spike-protein and that other therapeutically relevant target), or to provide a desired half-life, to provide a cytotoxic effect and/or to serve as a detectable tag or label. Some non-limiting examples of such other amino acid sequences, chemical entities or moieties are:
one or more suitable peptide or polypeptide linkers, such as a (G) nS, where n is 4 to 40;
one or more binding moieties, directed against a target other than spike-protein or epitope thereof, for example, against a different epitope of spike-protein;
one or more binding domains or binding units that provide for an increase in half-life (for example, a binding domain or binding unit that can bind against a serum protein;
a binding domain, binding unit or other chemical entity that allows for the spike-protein binder (e.g., an ISVD) to be internalized into a desired cell (for example, an internalizing anti-EGFR Nanobody® molecule as described in WO05044858);
a chemical moiety that improves half-life such as a suitable polyethyleneglycol group (i.e., PEGylation) or an amino acid sequence that provides for increased half-life such as human serum albumin or a suitable fragment thereof (i.e., albumin fusion);
a payload such as a cytotoxic payload;
a detectable label or tag, such as a radiolabel or fluorescent label;
a tag that can help with immobilization, detection and/or purification of the binder (e.g.,
an ISVD such as a Nanobody® ISVD), such as a HISn, wherein n is 6 to 18, or FLAG tag or combination thereof;
a tag that can be functionalized, such as a C-terminal GGC tag; or
a C-terminal extension X (n) (e.g.,-Ala), which may be as further described herein for the spike-protein binders (e.g., an ISVD such as a Nanobody® ISVD) of the invention and/or as described in WO12175741 or WO2015173325.

The invention further provides spike-protein binders that comprise a C-terminal extension. The invention provides, for example, C-terminal extensions such as X (n), wherein X comprises Ala, Gly, Val, Leu, and/or Ile and n can be 1 to 3.

In some embodiments, any C-terminal extension present in a spike-protein binder does not contain a free cysteine residue (unless said cysteine residue is used or intended for further functionalization, for example for PEGylation).

The spike-protein binders disclosed herein may also be conjugated to a chemical moiety. Such conjugated binders are an embodiment of the invention. The chemical moiety may be, inter alia, a polymer, a radionuclide, or a cytotoxic factor. In particular embodiments, the chemical moiety is a polymer that increases the half-life of the spike-protein binder in the body of a subject. Suitable polymers include, but are not limited to, hydrophilic polymers, which include but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The spike-protein binders disclosed herein may also be conjugated with labels such as ⁹⁹Tc, ⁹⁰Y, ¹¹¹ In, ³²p, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹ At, ²¹²Pb, ⁴⁷Sc, ¹⁰⁹Pd, ²³⁴Th, and ⁴⁰K, ¹⁵⁷Gd, ⁵⁵Mn, ⁵²Tr, and ⁵⁶Fe.

The spike-protein binders may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The spike-protein binder may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating a spike-protein binder to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating binders are conventional and very well known in the art.

The invention further provides nucleic acid molecules encoding any one of the spike-protein binders disclosed herein. In some embodiments, the nucleic acids (e.g., DNA) encode for the VHHs provided by SEQ ID NOs 1 to 24. In some embodiments, the nucleic acids (e.g., DNA) encode for a variable chain (e.g., of an antibody or a single-domain antibody) that comprises the CDR1, CDR2, and CDR3 of any row of Table D.

This invention also provides expression vectors comprising the isolated nucleic acids of the invention, wherein the nucleic acid is operably linked to control sequences that are recognized by a host cell when the host cell is transfected with the vector. Also provided are host cells comprising an expression vector of the invention and methods for producing the single-domain antibody, antibody, or antigen binding fragment thereof disclosed herein comprising culturing a host cell harboring an expression vector encoding the single-domain antibody, antibody, or antigen binding fragment thereof in culture medium, and isolating the single-domain antibody, antibody, or antigen binding fragment thereof from the host cell or culture medium.

### Compositions

To prepare pharmaceutical or sterile compositions of the spike-protein binders, the single-domain antibody, antibody, or antigen binding fragment thereof is admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the antibody compositions, administered alone or in combination with another agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). In particular aspects, antibodies exhibiting high therapeutic indices are desirable. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration.

In a further embodiment, a composition comprising a single-domain antibody, antibody, or antigen binding fragment thereof disclosed herein is administered to a subject in accordance with the Physicians' Desk Reference 2003 (Thomson Healthcare; 57th edition (Nov. 1, 2002)).

The mode of administration can vary. Some suitable routes of administration include transmucosal, intestinal, parenteral; intramuscular, subcutaneous, intradermal, intravenous, intraperitoneal, intranasal, inhalation, insufflation, topical, cutaneous, transdermal, or intra-arterial.

In particular embodiments, the spike-protein binder or antigen binding fragment thereof can be administered by an invasive route such as by injection (see above). In further embodiments of the invention, a spike-protein binder or antigen binding fragment thereof, or pharmaceutical composition thereof, is administered intravenously, subcutaneously, intramuscularly, intra-arterially, intra-articularly (e.g., in arthritis joints), or by inhalation, aerosol delivery. Administration by non-invasive routes (e.g., orally; for example, in a pill, capsule or tablet) is also within the scope of the invention.

Compositions can be administered with medical devices known in the art. For example, a pharmaceutical composition of the invention can be administered by injection with a hypodermic needle, including, e.g., a prefilled syringe or autoinjector.

The pharmaceutical compositions disclosed herein may also be administered by infusion.

Alternately, one may administer the antibody in a local rather than systemic manner, for example, via injection of the single-domain antibody, antibody, or antigen binding fragment thereof directly into an arthritic joint or pathogen-induced lesion characterized by immunopathology, often in a depot or sustained release formulation.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies may be desirable.

Further provided are kits comprising one or more components that include, but are not limited to, a single-domain antibody, antibody, or antigen binding fragment, as discussed herein, which binds the spike protein in association with one or more additional components including, but not limited to a pharmaceutically acceptable carrier and/or another therapeutic agent, as discussed herein.

In one embodiment, the kit includes a composition of the invention (e.g., a spike protein binder) or a pharmaceutical composition thereof in one container (e.g., in a sterile glass or plastic vial) and a pharmaceutical composition thereof and/or another therapeutic agent in another container (e.g., in a sterile glass or plastic vial).

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely.

The kit can further include a second therapeutic agent or vaccine.

### Methods and Uses

The spike-protein binders disclosed herein can be used in a variety of ways. In some embodiments, one administers to a subject in need thereof an effective amount of the polypeptide or single-domain antibody of any one of the disclosed embodiments to treat a coronavirus infection (e.g., by SARS-CoV, SARS-CoV2, or MERS-CoV) or a coronavirus disease (e.g., SARS, MERS, or COVID-19) in the subject.

In some embodiments, the polypeptide or single-domain antibody of any one of the disclosed embodiments is for the treatment of a coronavirus infection (e.g., by SARS-CoV, SARS-CoV2, or MERS-CoV) or a coronavirus disease (e.g., SARS, MERS, or COVID-19) or is for the preparation of a medicament for such a treatment.

Since the polypeptides and single-domain antibodies of the disclosed embodiments were created to bind to multiple spike proteins from different coronaviruses, they can potentially be used against future coronaviruses that are yet to emerge.

The spike-protein binders disclosed herein may be used as affinity purification agents. In this process, the spike-protein binders are immobilized on a solid phase such a Sephadex resin or filter paper, using methods well known in the art. The immobilized spike-protein binder is contacted with a sample containing the spike protein (or fragment thereof) to be purified, and thereafter the support is washed with a suitable solvent that will remove substantially all the material in the sample except the spike protein, which is bound to the immobilized antibody or fragment. Finally, the support is washed with a solvent which elutes the bound spike protein from the column (e.g., protein A). Such immobilized spike-protein binders form part of the invention.

Further provided are antigens for generating secondary antibodies which are useful for example for performing Western blots and other immunoassays discussed herein. in particular, polypeptides are disclosed which comprise the variable regions and/or CDR sequences of a therapeutic spike-protein binders disclosed herein, and which may be used to generate an anti-idiotypic antibodies for use in specifically detecting the presence of the antibody, e.g., in a therapeutic context.

Anti-target antibodies or fragments thereof may also be useful in diagnostic assays for the target protein, e.g., detecting its expression in specific cells, tissues, or serum. Such diagnostic methods may be useful in various disease diagnoses. For example, particular embodiments include ELISA assays (enzyme-linked immunosorbent assay) incorporating the use of an spike-protein binder or fragment thereof disclosed herein. An anti-target antibody of the invention may be used in a Western blot or immune-protein blot procedure.

The spike-protein binders and antigen-binding fragments thereof disclosed herein may also be used for immunohistochemistry. Such a method forms part of the invention and comprises, e.g., (1) contacting a cell to be tested for the presence of target with a spike-protein binder or antigen-binding fragment thereof of the invention; and (2) detecting the spike-protein binder or fragment on or in the cell. If the spike-protein binder or fragment itself is detectably labeled, it can be detected directly. Alternatively, the antibody or fragment may be bound by a detectably labeled secondary antibody which is detected.

Standard methods in molecular biology are described in Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA. Standard methods also appear in Ausubel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (e.g., Coligan, et al. (2000) Current Protocols in Protein Science, Vol. 1, John Wiley and Sons, Inc., New York). Chemical analysis, chemical modification, post-translational modification, production of fusion proteins, and glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (e.g., Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2nd ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (e.g., Molecular Probes (2003) Catalogue, Molecular Probes, Inc., Eugene, OR; Sigma-Aldrich (2003) Catalogue, St. Louis, MO).

### Exemplification

The following examples are provided to promote a further understanding of the invention.

### Example 1: Discovery and Multimerization of Cross-Reactive Single-Domain Antibodies Against SARS-Like Viruses to Enhance Potency and Address Emerging SARS-CoV-2 Variants

Below, we report the discovery of multiple VHHs that bind distinct cross-reactive epitopes on the spike protein. We further demonstrate that the neutralization potencies of multimeric VHHs are greatly enhanced compared to the monovalent forms. The enhanced potency and potential to protect against escape mutations make this approach valuable in addressing emerging SARS-CoV-2 variants as well as SARS-like viruses that may emerge in the future.

### Identification of Cross-Reactive VHHs

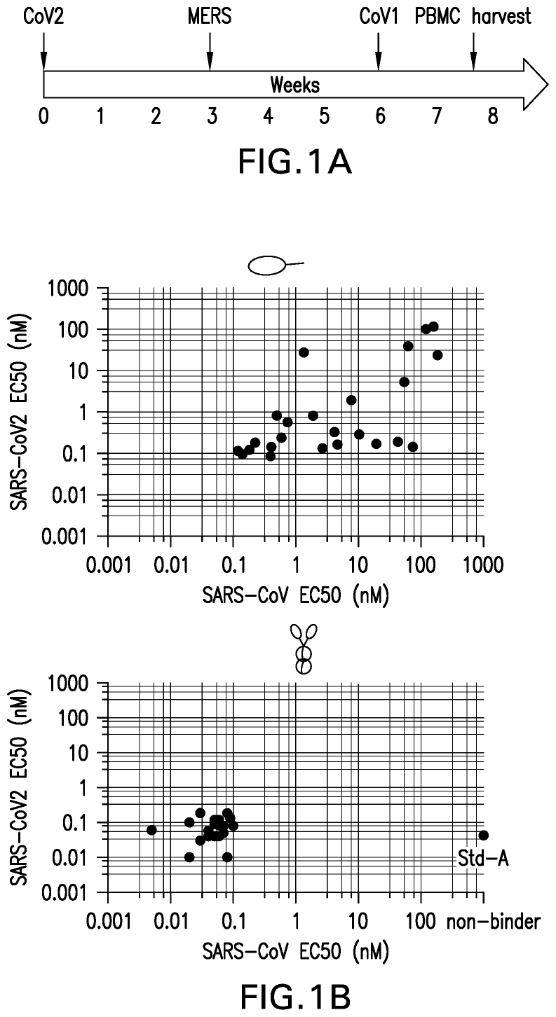

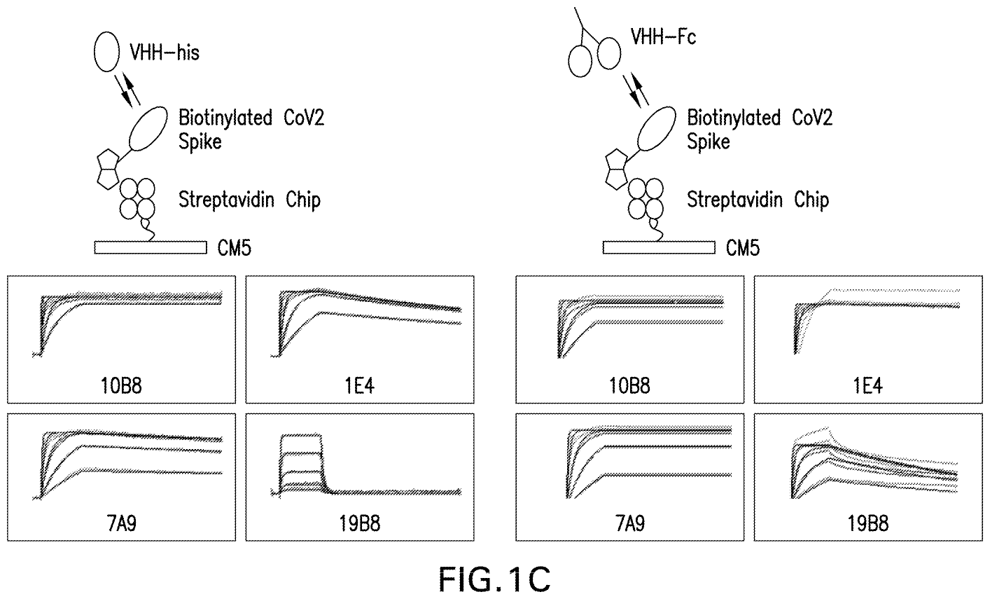

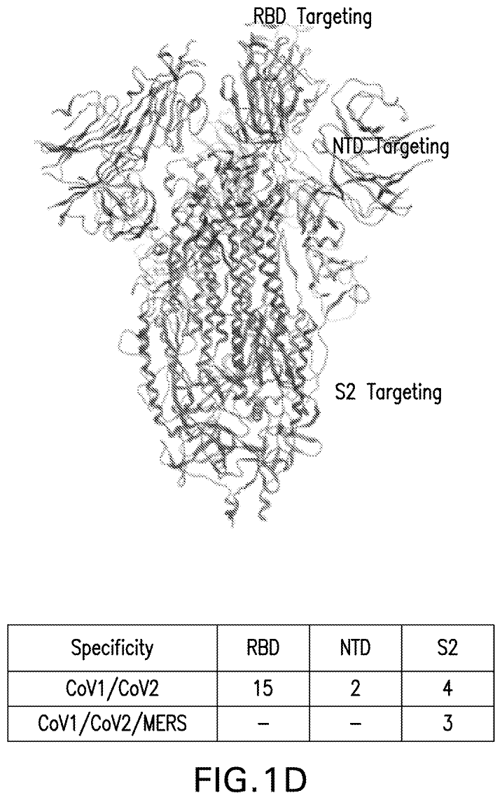

We used a heterologous immunization approach to identify heavy-chain variable domains (VHHs) with broad cross-reactivity (FIG. 1A). We hypothesized that cross-reactive B cells will be enriched after three immunizations with related but not identical spike proteins. To that end, llamas were first immunized with SARS-CoV2 spike protein followed by spike proteins from MERS and SARS-CoV viruses with three-week intervals between the immunizations. Peripheral blood mononuclear cells (PBMCs) were harvested 10 days after the final immunization. B cells binding to SARS-CoV-2 spike protein were isolated and cultured, and supernatants were used to screen for binding to spike proteins from the three viruses. Twenty-four clones that bound to both SARS-CoV and SARS-CoV2 spikes were sequenced, recombinantly expressed in either monomeric or bivalent formats, and validated for binding by ELISA (FIG. 1B, Table 1) and flow cytometry (Table 2). All 24 constructs bound with similar affinity to SARS-CoV and SARS-CoV-2 with significant affinity improvement observed in the bivalent format (Table 3). Many bivalent VHHs showed affinity to SARS-CoV-2 spike similar to that of a potent benchmark antibody Std-A. Binding affinity mostly remained unchanged for SARS-CoV2 variants Alpha, Beta, Gamma, and Delta. However, 8 of the candidates lost binding to the Omicron variants BA.1 and BA.2 (Table 1). In addition, a subset the 24 VHHs also bound to spike proteins from MERS and endemic beta human coronaviruses OC43 and HKU1 (Table 1). Direct affinity measurement using Biacore™ (FIG. 1C) validated ELISA results. Candidates were further evaluated for binding to different domains of the spike protein (RBD, NTD, S2) using ELISA (FIG. 1D). Using this approach, we were able to identify SARS-CoV and SARS-CoV-2 double cross-reactive clones targeting all 3 domains of the spike protein. In contrast, triple cross-reactive clones that bound to all three spike proteins (SARS-CoV, SARS-CoV-2, MERS) were restricted to the S2 domain.

TABLE 1
ELISA binding of VHH-Fcs (EC50 values)

| | Binding EC50 (nM) | | | | | | | | | | |
|---|---|---|---|---|---|---|---|---|---|---|---|
| Clone | WT | Alpha \| | Beta \| | Gamma \| | Delta \| | Omicron | Omicron \| | WT | | | |
| Name | Cov2 | B.1.1.7 | B.1.351 | P.1 | B.1.617.2 | \|BA1.1 | BA.2 | Cov1 | MERS | OC43 | HKU1 |
| 8E9 | 0.08 | 0.06 | 0.10 | 0.15 | 0.06 | 0.60 | 0.60 | 0.08 | NB | NA | NA |
| 9B2 | 0.10 | 0.06 | 0.07 | 0.05 | 0.06 | 0.18 | NB | 0.12 | NB | NA | NA |
| 11F8 | 0.19 | 0.08 | 0.21 | 0.14 | 0.11 | NB | NB | 0.18 | NB | NA | NA |
| 13C8 | 0.09 | 0.11 | 0.24 | 0.23 | 0.18 | NB | NB | 0.10 | NB | NA | NA |
| 15H1 | 0.14 | 0.05 | 0.10 | 0.09 | 0.10 | NB | NB | 0.06 | NB | NA | NA |
| 15H7 | 0.05 | 0.05 | 0.07 | 0.06 | 0.06 | NB | NE | 0.08 | NE | NA | NA |
| 1A11 | 0.18 | 0.04 | 0.62 | 0.24 | 0.20 | 0.64 | 0.64 | 0.05 | NB | NA | NA |
| 1E4 | 0.04 | 0.62 | 0.03 | 0.03 | 0.06 | NB | NB | 0.04 | NB | NA | NA |
| 19E10 | 0.03 | 0.06 | 0.05 | 0.03 | 0.04 | 0.09 | 0.09 | 0.03 | NB | NA | NA |
| 5D7 | 0.11 | 0.74 | 0.22 | 0.09 | 0.06 | 0.22 | 0.22 | 0.04 | NB | NA | NA |
| 10B8 | 1.35 | 0.05 | 0.03 | 1.11 | 0.85 | 2.81 | 2.81 | 0.13 | NB | NA | NA |
| 3D4 | 0.13 | 0.11 | 0.09 | 0.09 | 0.08 | NB | NE | 0.12 | NB | NA | NA |
| 5A6 | 0.14 | 0.11 | 0.15 | 0.10 | 0.08 | NB | NE | 0.09 | NB | NA | NA |
| 19C10 | 0.06 | 0.18 | 0.04 | 0.14 | 0.10 | NB | NB | 0.08 | NB | NA | NA |
| 7A9 | 0.16 | 0.13 | 0.13 | 0.12 | 0.17 | 0.19 | 0.19 | 0.06 | NB | NA | NA |
| 19B8 | 0.06 | 0.63 | 0.26 | 0.17 | 0.08 | 0.04 | 0.04 | 0.04 | NB | NA | NA |
| 16H7 | 0.08 | 0.06 | 0.18 | 0.04 | 0.62 | 0.03 | 0.51 | 0.18 | NB | NA | NA |
| 20D11 | 0.62 | 0.03 | 0.06 | 0.56 | 0.69 | 0.53 | 0.53 | 0.04 | NB | NA | NA |
| 5G11 | 0.40 | 0.22 | 0.23 | 0.21 | 0.36 | 0.26 | 0.26 | 0.18 | NB | NA | NA |
| 11F5 | 0.12 | 0.05 | 0.06 | 0.05 | 0.05 | 0.04 | 0.04 | 0.09 | NB | NA | NA |
| 14F3 | 0.05 | 0.03 | 0.08 | 0.03 | 0.03 | 0.03 | 0.03 | 0.05 | NB | NA | NA |
| 6A1 | 0.44 | 0.20 | 0.20 | 0.18 | 0.27 | 0.20 | 0.20 | 0.10 | 0.08 | 0.08 | 1.60 |
| S3_44 | WB | WB | WB | WB | WB | WB | WB | WB | NB | WB | NA |
| S3_29 | WB | WB | WB | WB | WB | WB | WB | WB | NB | WB | NA |
| Std-D | 0.04 | 0.06 | 0.09 | 0.07 | 0.06 | NB | NB | NA | NA | NA | NA |
| Std-A | 0.09 | 0.10 | 0.13 | 0.13 | 0.15 | NB | NB | NA | NA | NA | NA |
| Std-B | 0.10 | 0.09 | 8.28 | 6.50 | 0.15 | NB | NB | NA | NA | NA | NA |
| Std-C | 0.40 | 0.33 | 0.69 | 0.61 | 0.43 | 9.94 | 9.94 | NA | NA | NA | NA |

NB No binding
WB Weakly binding,
EC50 not calculated
NA Not available

TABLE 2
FACS binding of VHH-Fcs (EC50 values)

Binding EC50 (nM)

| Clone | WT | WT | |
|---|---|---|---|
| Name | Cov2 | Cov1 | MERS |
| 8E9 | 0.31 | 0.17 | NB |
| 9B2 | 0.21 | 0.13 | NB |
| 11F8 | 0.08 | 0.09 | NB |
| 13C8 | 0.22 | 0.11 | NB |
| 15H1 | 0.25 | 0.14 | NB |
| 15H7 | 0.28 | 0.14 | NB |
| 1A11 | 0.13 | 0.06 | NB |
| 1E4 | 0.06 | 0.06 | NB |
| 19E10 | 0.14 | 0.08 | NB |
| 5D7 | 0.12 | 0.09 | NB |
| 10B8 | 0.19 | 0.14 | NB |
| 3D4 | 0.15 | 0.12 | NB |
| 5A6 | 0.43 | 0.21 | NB |
| 19C10 | 0.17 | 0.13 | NB |
| 7A9 | 0.08 | 0.05 | NB |
| 19B8 | 0.04 | 0.06 | NB |
| 16H7 | 0.31 | 0.10 | NB |
| 20D11 | 0.08 | 0.10 | NB |
| 5G11 | NA | NA | NA |
| 11F5 | 0.04 | 0.05 | NB |
| 14F3 | 0.06 | 0.09 | NB |
| 6A1 | 0.14 | 0.19 | 0.13 |
| S3_44 | 0.08 | 0.09 | 0.11 |
| S3_29 | 0.03 | 0.04 | 0.05 |

NB No binding
NA Not available

TABLE 3
Apparent affinities of VHH and VHH-
Fcs to SARS-CoV2 spike protein

| | |
|---|---|
| Clone | Affinity KD (M) |

| Name | VHH | VHH-Fc |
|---|---|---|
| 8E9 | 5.7E−09 | <2.0E−10 |
| 9B2 | 6.1E−10 | <2.0E−10 |
| 11F8 | 9.5E−10 | <2.0E−10 |
| 13C8 | 1.2E−08 | <2.0E−10 |
| 15H1 | 6.0E−10 | <2.0E−10 |
| 15H7 | 3.3E−10 | <2.0E−10 |
| 1A11 | <2.0E−10 | <2.0E−10 |
| 1E4 | 2.5E−10 | <2.0E−10 |
| 19E10 | 7.3E−10 | <2.0E−10 |
| 5D7 | 1.5E−09 | <2.0E−10 |
| 10B8 | <2.0E−10 | <2.0E−10 |
| 3D4 | <2.0E−10 | <2.0E−10 |
| 5A6 | 4.6E−10 | <2.0E−10 |
| 19C10 | <5.0E−08 | <2.0E−10 |
| 7A9 | 2.2E−10 | <2.0E−10 |
| 19B8 | 8.7E−08 | 1.1E−09 |
| 16H7 | 2.3E−07 | 3.0E−09 |
| 20D11 | 2.2E−09 | <2.0E−10 |
| 5G11 | 9.3E−08 | 1.5E−09 |
| 11F5 | 8.0E−09 | <2.0E−10 |
| 14F3 | 3.0E−09 | <2.0E−10 |
| 6A1 | >5.0E−07 | 1.6E−09 |
| S3_44 | NB | NB |
| S3_29 | NB | NB |

NB No binding

### Potent Cross-Neutralization of SARS-CoV and SARS-CoV2

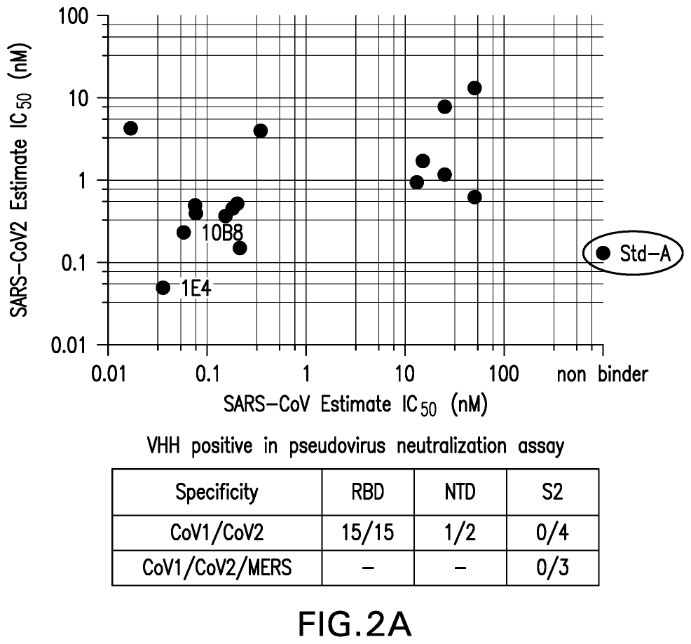

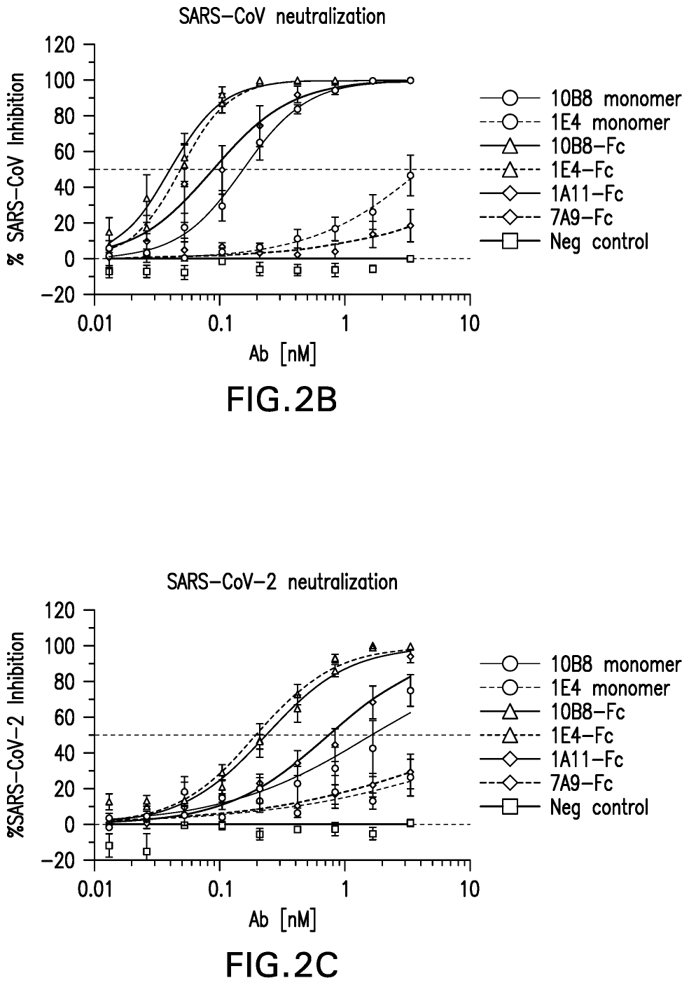

We initially evaluated neutralization potency of candidate VHHs (i.e., ability to inhibit infection) in the bivalent format using VSV-based pseudovirus neutralization assays and observed cross-neutralization of SARS-CoV and SARS-CoV-2 with several candidates (FIG. 2A, Table 4). In particular, 8 of the candidates, including 10B8 and 1E4, showed potent neutralization for both SARS-CoV and SARS-CoV2. This contrasts with the Std-A antibody that only neutralizes SARS-CoV2. Neutralization was observed with all RBD-binding and some NTD-binding antibodies. None of the S2-binding antibodies showed neutralization in SARS-CoV, SARS-CoV2, and MERS pseudovirus assays (Table 4). Importantly, neutralization potency remained similar for the SARS-CoV2 variants Alpha, Beta and Delta. While potency was reduced against the Omicron variant for some candidates, it remained high for several-particularly 10B8 (Table 4).

TABLE 4
Pseudovirus neutralization potency of VHH-Fcs

| | Neutralization potency IC50 (nM) | | | | | | |
|---|---|---|---|---|---|---|---|
| Clone | WT | WT | | Alpha \| | Beta \| | Gamma \| | Omicron |
| Name | Cov2 | Cov1 | MERS | B.1.1.7 | B.1.351 | P.1 | \|BA1.1 |
| 8E9 | 12.4 | 2.94 | NN | NA | NA | NA | 429 |
| 9B2 | 1.93 | 0.19 | NN | 22 | 4.2 | 21 | 52 |
| 11F8 | 1.07 | 206.8 | NN | NA | NA | NA | NN |
| 13C8 | 6.69 | 4.98 | NN | NA | NA | NA | NA |
| 15H1 | 5.04 | 12.31 | NN | NA | NA | NA | NA |
| 15H7 | 1.33 | 1.71 | NN | NA | NA | NA | NA |
| 1A11 | 0.57 | 0.18 | NN | 6.6 | 4.1 | 6.4 | 42 |
| 1E4 | 0.31 | 0.06 | NN | 2.2 | 0.58 | 2.6 | NA |
| 19E10 | 1.53 | 0.12 | NN | 7 | 3.1 | 5.9 | 309 |
| 5D7 | 6.52 | 220.3 | NN | NA | NA | NA | 221 |
| 10B8 | 0.68 | 0.09 | NN | 0.38 | 0.23 | 0.21 | 4.1 |
| 3D4 | 1.22 | 0.40 | NN | 1.5 | 1.1 | 0.59 | NA |
| 5A6 | 8.57 | 6.30 | NN | 26 | 54 | 3.1 | NA |
| 19C10 | 1.49 | 0.45 | NN | 0.85 | 0.66 | 0.8 | NA |
| 7A9 | 110 | WN | NN | NA | NA | NA | 44 |
| 19B8 | 53.45 | 42.03 | NN | NA | NA | NA | 47 |
| 16H7 | NN | NN | NN | NA | NA | NA | NA |
| 20D11 | NN | NN | NN | NA | NA | NA | NA |
| 5G11 | NN | NN | NN | NA | NA | NA | NA |
| 11F5 | NN | NN | NN | NA | NA | NA | NA |
| 14F3 | NN | NN | NN | NA | NA | NA | NA |
| 6A1 | NN | NN | NN | NA | NA | NA | NA |
| S3_44 | NN | NN | NN | NA | NA | NA | NA |
| S3_29 | NN | NN | NN | NA | NA | NA | NA |
| Std-A | 1.9 | NN | NA | 0.39 | 1.7 | 0.75 | NA |
| Std-F | 5.0 | NA | NA | NA | NA | NA | NN |
| Std-C | NN | NA | NA | NA | NA | NA | 645 |

NN Non-neutralizing
WN Weakly-neutralizing,
IC50 not calculated

Next, neutralization potencies of select candidates were evaluated in SARS-CoV and SARS-CoV-2 authentic virus assays. Overall neutralization potency remained similar to what was observed in pseudovirus assays, with two of the potent candidates 10B8 and 1E4 showing IC50 values at sub-nM levels for both SARS-CoV and SARS-CoV-2 (FIG. 2B and FIG. 2C). The bivalent format of 10B8 showed IC50 values of 40 pM and 300 pM for SARS-CoV and SARS-CoV2, respectively. Even in the monovalent format, IC50 values of 10B8 remained potent (150 pM for SARS-CoV and 2.9 nM for SARS-CoV2). While the bivalent format of 1E4 remained potent (IC50 values of 50 pM and 200 pM for SARS-CoV and SARS-CoV2 respectively), the monovalent format of this construct was less potent and IC50 values could not be calculated with the concentrations used in the experiment.

### Epitope Mapping and Structural Analysis

We used an Octet-based binning assay to evaluate the epitopes of candidate VHHs. Briefly, the first VHH was incubated with the Cov-2 spike protein, which was captured on the biosensor tip, followed by binding of a second antibody. Several benchmark antibodies against the RBD (Std-B, Class 1; Std-A, Class 3; Std-C, Class 3; Std-D, Class 4; and Std-G, Class 4) were included in some experiments, for example to classify the RBD-targeting VHHs we discovered. Strikingly, fourteen out of 15 RBD binders binned with Std-D, which binds to a cryptic cross-reactive Class 4 epitope on the RBD (Table 5A). In contrast, one RBD binder (7A9) did not bin with any of the other candidates tested (Class 1, 3 or 4), indicating a novel epitope. We did not find any cross-reactive RBD-targeting VHHs that bound to either the Class 1 or Class 2 epitopes, consistent with literature that epitopes on the top of the RBD are more variable and strain-specific. Likewise, both NTD binding VHHs (19B8 and 16H7) binned together (Table 5B). With regard to S2 binders, four of the VHHs that did not bind to MERS binned together, while one triple cross-reactive VHH (6A1) binned separately from the four double cross-reactive VHHs, indicating that they indeed target distinct epitopes on the S2 domain. The other two triple cross-reactive VHHs were not evaluated in this assay since they bound well to cell surface expressed spike but poorly to recombinant proteins.

TABLE 5A
Binning of candidate VHHs-RBD binders

| | Antibody 1 bound to SARS-CoV-2 Spike ECD | | | | | | | | | | |
|---|---|---|---|---|---|---|---|---|---|---|---|
| | | | | | | | | | | | RBD |
| | RBD | RBD | | | | | | | | | Class |
| | Class | Class | | | | | | | | | (not |
| Antibody 2 | 1 | 3 | RBD Class 4 | | | | | | | | 1/3/4) |
| flowed | Std-B | Std-A | Std-D | 8E9 | 9B2 | 11F8 | 13C8 | 15H1 | 15H7 | 1A11 | 7A9 |
| Std-D | 0.25 | 0.25 | 0.03 | −0.01 | −0.01 | −0.01 | 0.00 | −0.01 | −0.02 | −0.04 | 0.19 |
| 8E9 | 0.34 | 0.36 | 0.04 | 0.08 | 0.11 | 0.11 | 0.11 | 0.13 | 0.11 | 0.09 | 0.39 |
| 9B2 | 0.32 | 0.32 | 0.05 | 0.09 | 0.10 | 0.11 | 0.12 | 0.13 | 0.11 | 0.10 | 0.36 |
| 11F8 | 0.29 | 0.17 | 0.08 | 0.12 | 0.13 | 0.14 | 0.15 | 0.15 | 0.14 | 0.12 | 0.35 |
| 13C8 | 0.31 | 0.34 | 0.04 | 0.09 | 0.11 | 0.11 | 0.10 | 0.11 | 0.10 | 0.11 | 0.37 |
| 15H1 | 0.30 | 0.35 | 0.05 | 0.08 | 0.08 | 0.09 | 0.10 | 0.11 | 0.11 | 0.08 | 0.35 |
| 15H7 | 0.32 | 0.36 | 0.08 | 0.06 | 0.08 | 0.11 | 0.08 | 0.09 | 0.10 | 0.09 | 0.37 |
| 1A11 | 0.34 | 0.35 | 0.06 | 0.10 | 0.13 | 0.13 | 0.11 | 0.14 | 0.14 | 0.10 | 0.38 |
| 1E4 | 0.24 | 0.28 | 0.08 | 0.09 | 0.11 | 0.11 | 0.12 | 0.11 | 0.11 | 0.09 | 0.26 |
| 19E10 | 0.22 | 0.26 | 0.06 | 0.09 | 0.11 | 0.12 | 0.11 | 0.12 | 0.11 | 0.08 | 0.30 |
| 5D7 | 0.25 | 0.28 | 0.12 | 0.10 | 0.12 | 0.12 | 0.15 | 0.16 | 0.15 | 0.12 | 0.31 |
| 10B8 | 0.36 | 0.39 | 0.05 | 0.09 | 0.12 | 0.10 | 0.12 | 0.12 | 0.12 | 0.10 | 0.42 |
| 3D4 | 0.31 | 0.34 | 0.04 | 0.08 | 0.09 | 0.09 | 0.10 | 0.10 | 0.09 | 0.08 | 0.32 |
| 5A6 | 0.35 | 0.39 | 0.03 | 0.09 | 0.11 | 0.09 | 0.09 | 0.12 | 0.10 | 0.09 | 0.37 |
| 19C10 | 0.35 | 0.35 | 0.04 | 0.10 | 0.11 | 0.12 | 0.10 | 0.10 | 0.10 | 0.08 | 0.36 |
| 7A9 | 0.30 | 0.34 | 0.46 | 0.37 | 0.38 | 0.32 | 0.52 | 0.37 | 0.34 | 0.32 | 0.06 |

TABLE 5B
Binning of candidate VHHs-NTD and S2 binders

Antibody 1 bound to SARS-
CoV-2 Spike ECD

| | | |
|---|---|---|
| Antibody 2 | S2 | NTD |

| flowed | 20D11 | 6A1 | 19B8 |
|---|---|---|---|
| 20D11 | 0.04 | 0.41 | 0.43 |
| 5G11 | 0.05 | NT | 0.40 |
| 11F5 | 0.10 | NT | 0.52 |
| 14F3 | 0.04 | NT | 0.46 |
| 6A1 | 0.37 | 0.09 | 0.40 |
| 19B8 | 0.48 | NT | 0.08 |
| 16H7 | 0.56 | NT | 0.19 |

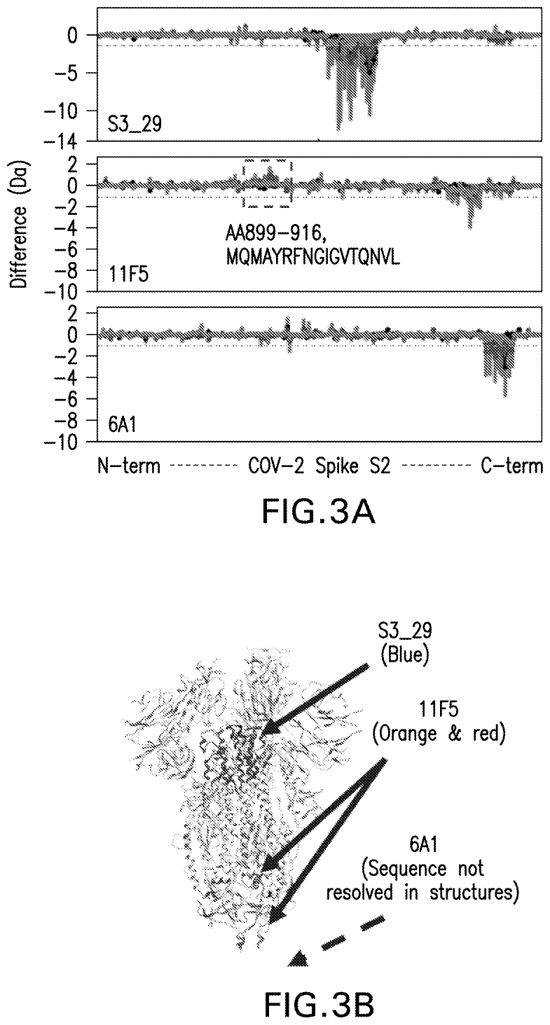

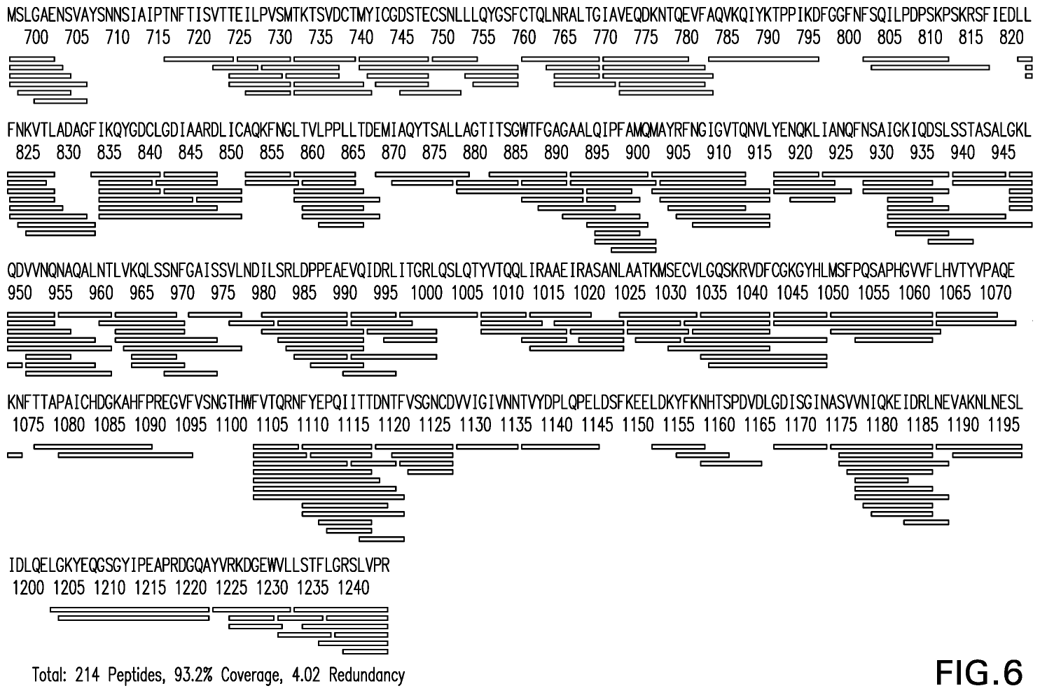

Unlike the well documented cross-reactive antibodies targeting the RBD, benchmark S2-binding antibodies with known epitopes are scarce. Therefore, we next utilized hydrogen-deuterium exchange mass spec (HDX-MS) to map epitopes of the three classes of S2 binders we identified. These included 6A1 (triple cross-reactive), 11F5 (double cross-reactive), and S3-29 which was triple cross-reactive in the FACS binding assay (FIG. 3A, FIG. 3B, FIG. 6). Our HDX data revealed three distinct binding epitopes for these S2-targeting VHHs with high confidence. The first epitope (shown in blue, FIG. 3B) is located at the top of the S2 domain and targeted by the triple cross-reactive VHH S3_29. The second S2 epitope is located at the stem-helix region at the base of the spike protein and targeted by the triple cross-reactive VHH 6A1. The final epitope is also located at the base of the spike protein but above the stem helix region (shown in orange and red, FIG. 3B), targeted by the double cross-reactive VHH 11F5 and likely by a few other VHHs that bin together with it (Table 5B).

### VHH 7A9 Binds a Rare RBD Epitope and Destabilizes the Spike Trimer

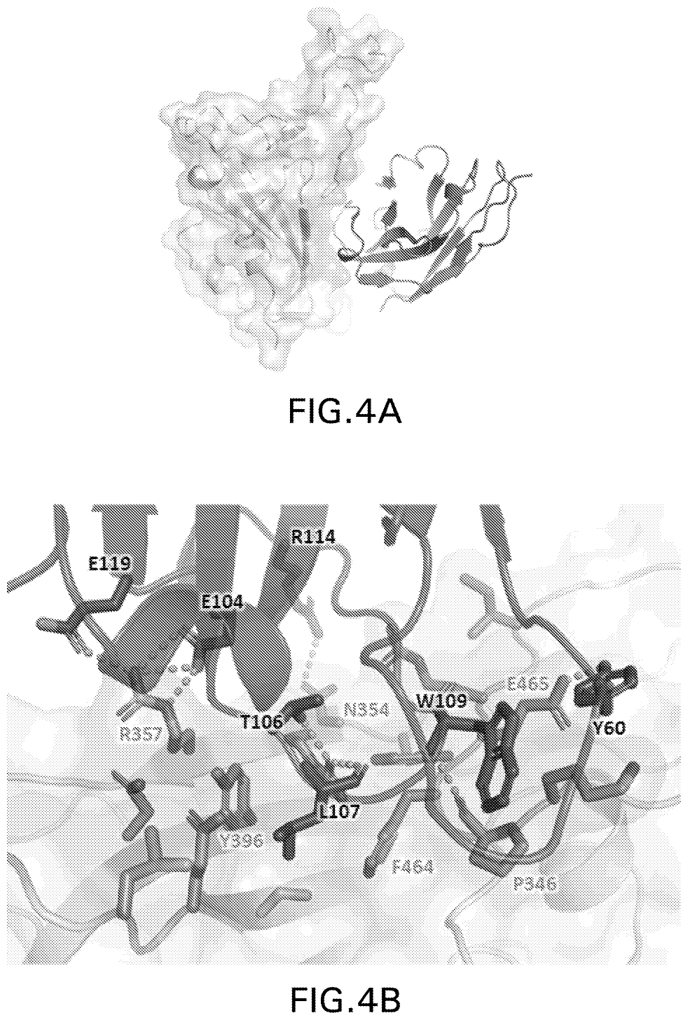

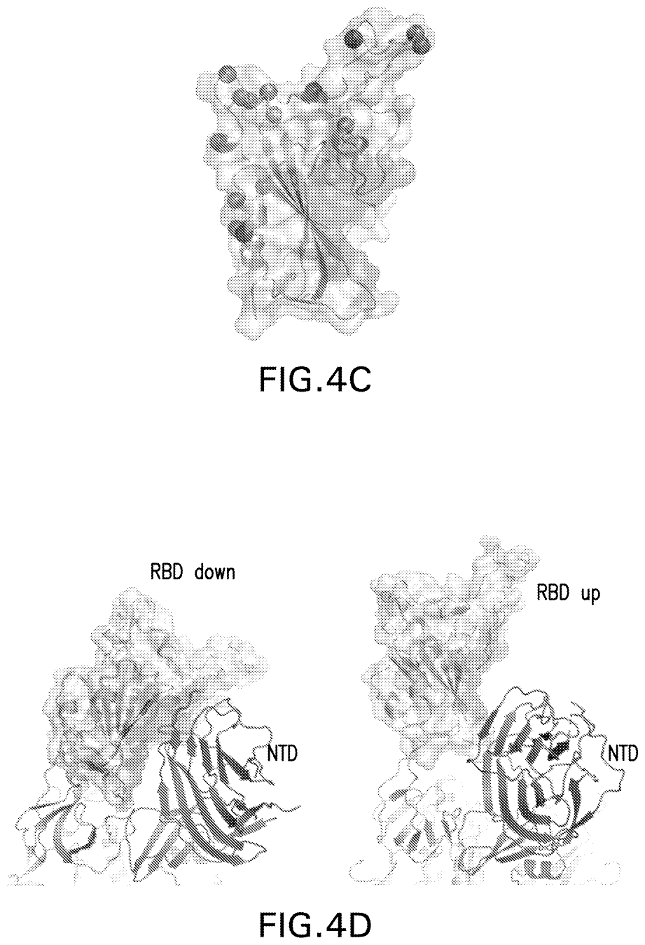

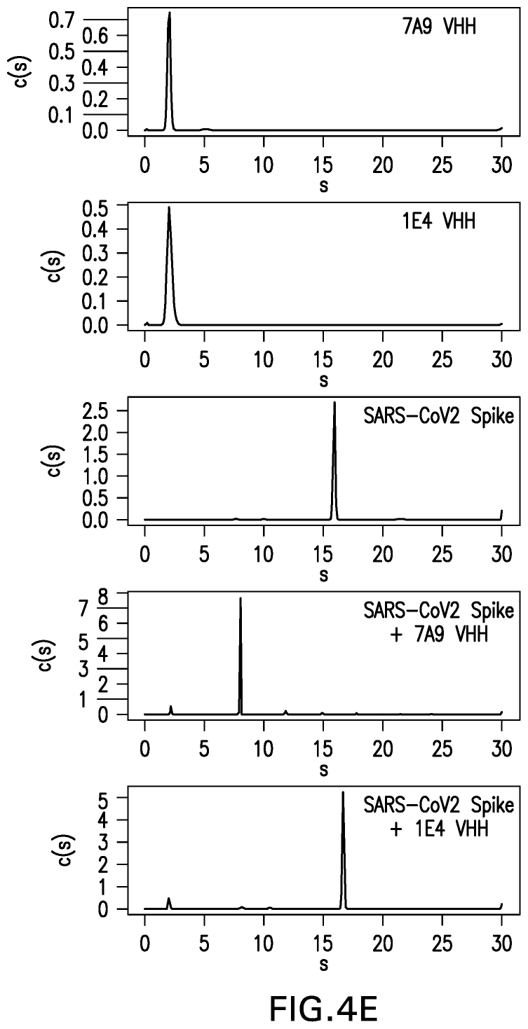

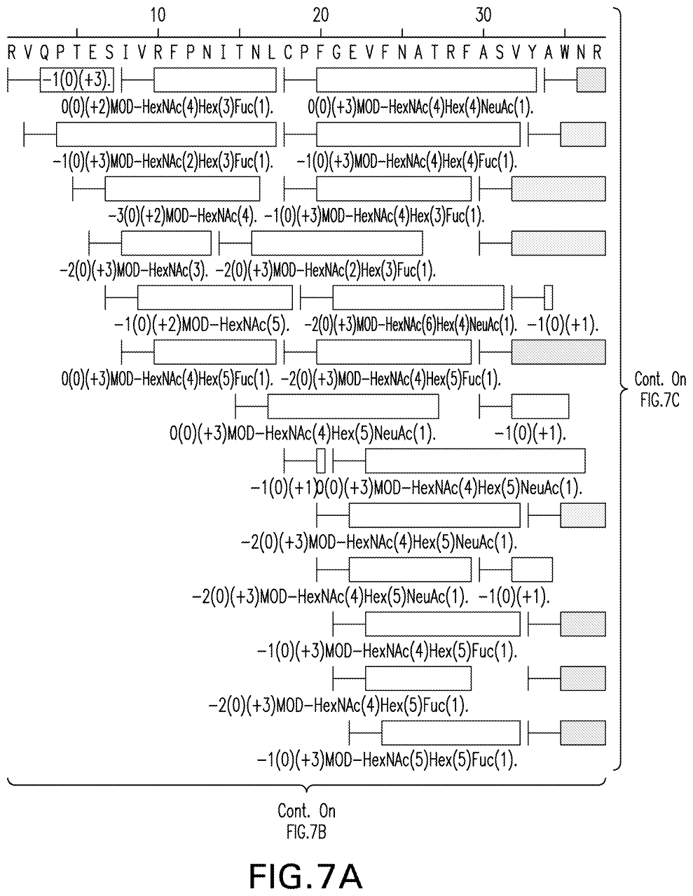

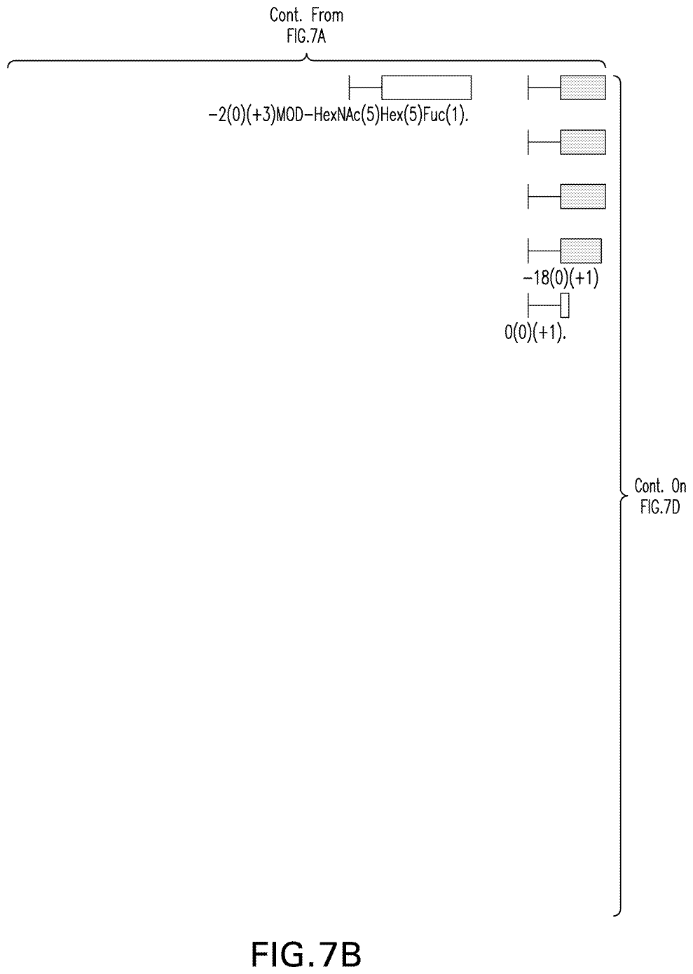

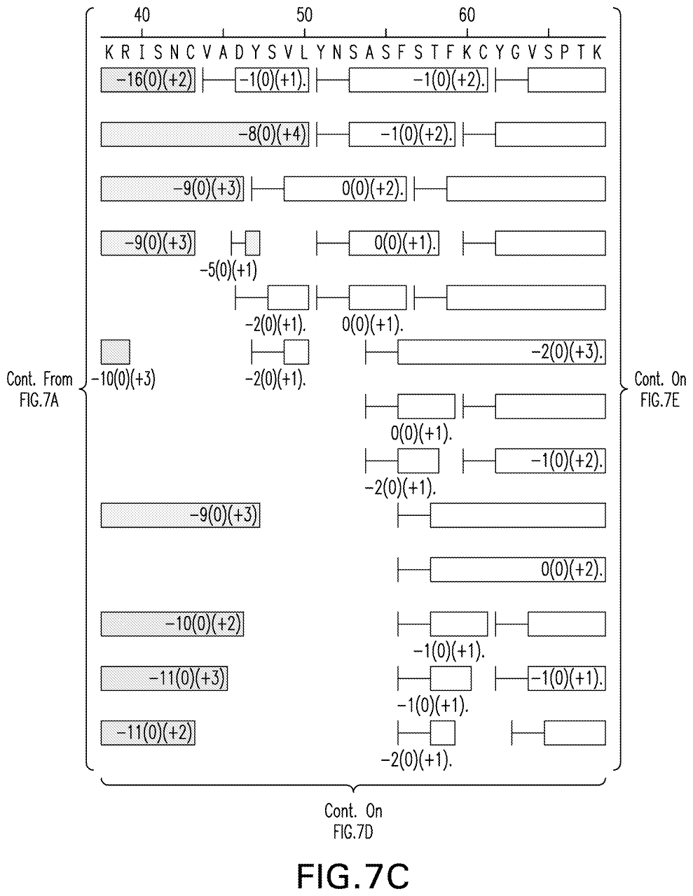

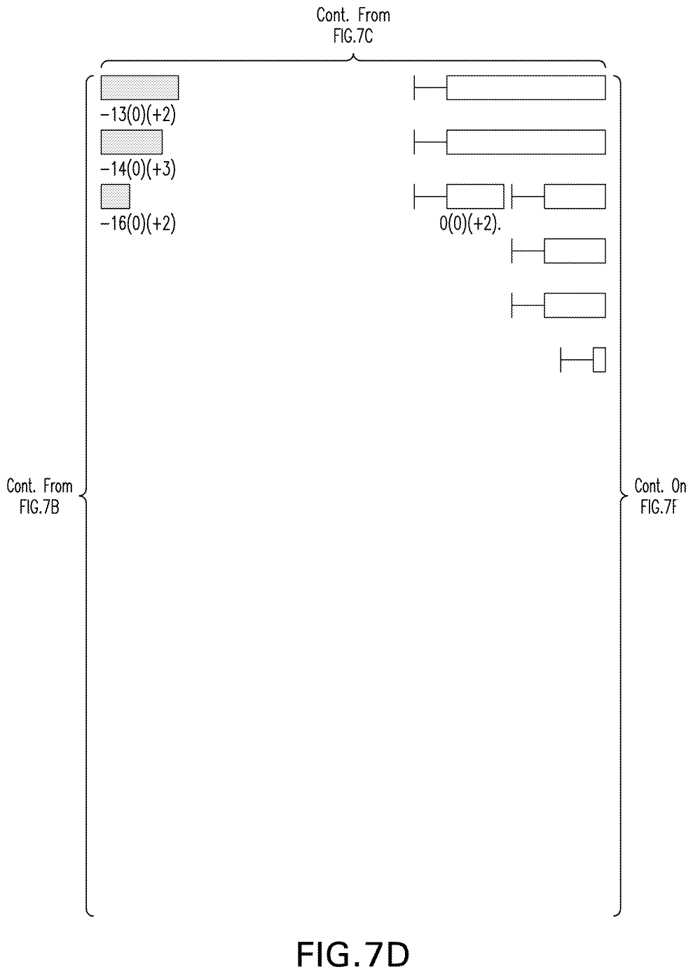

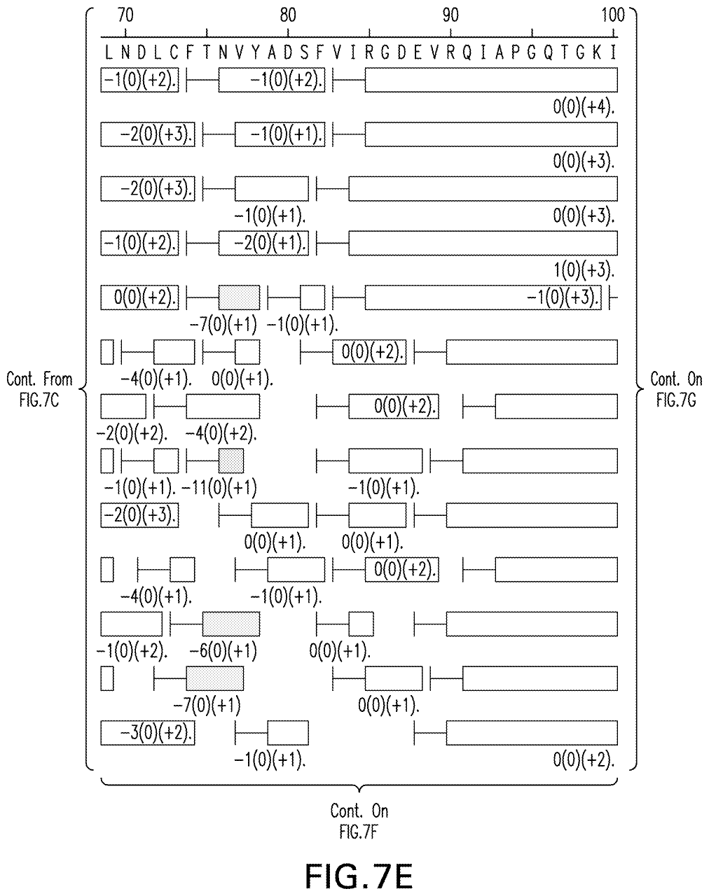

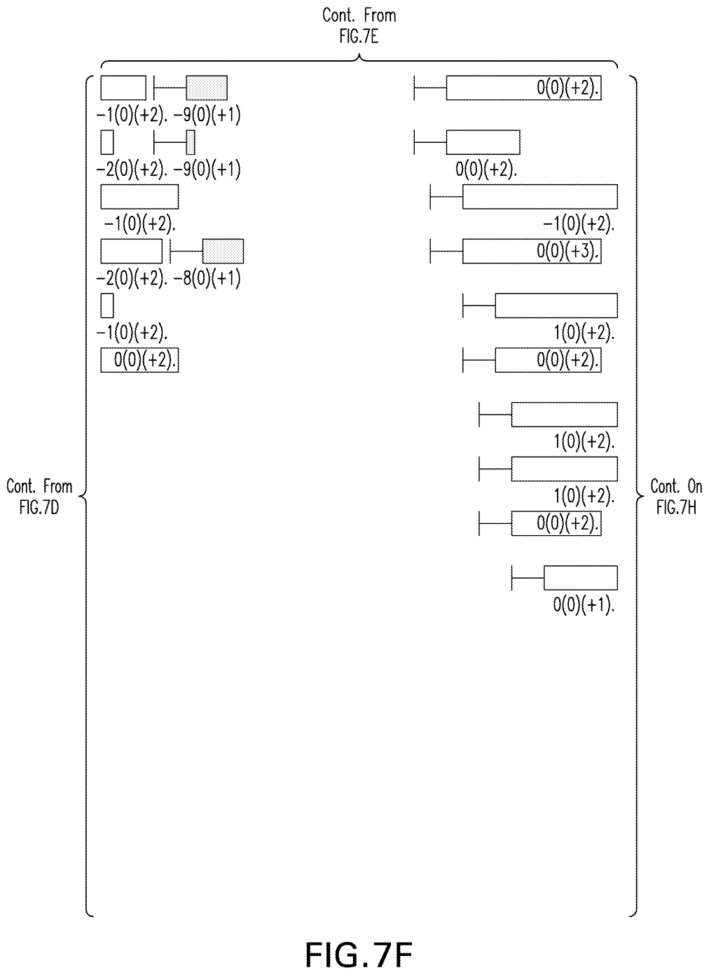

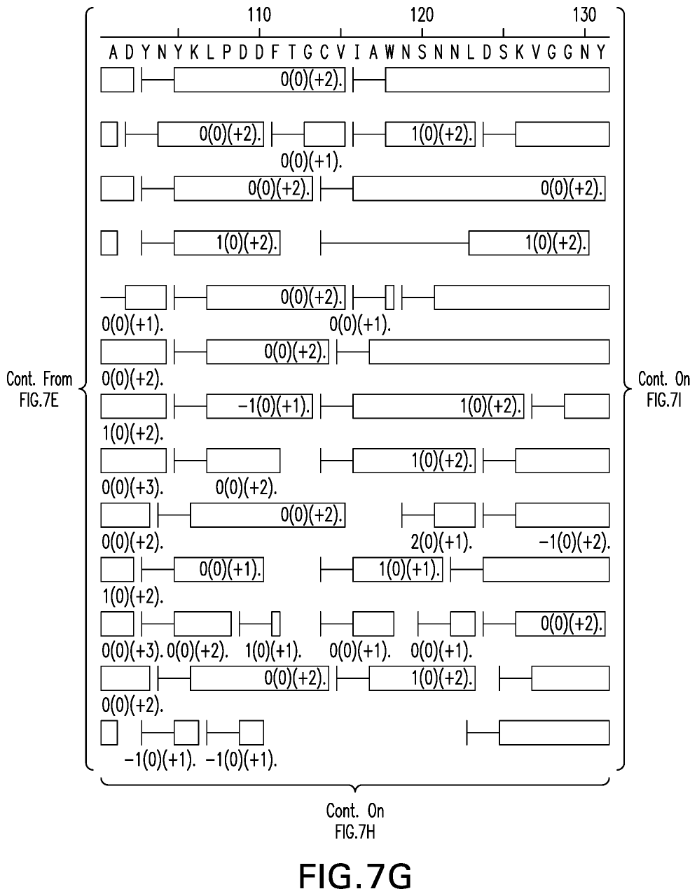

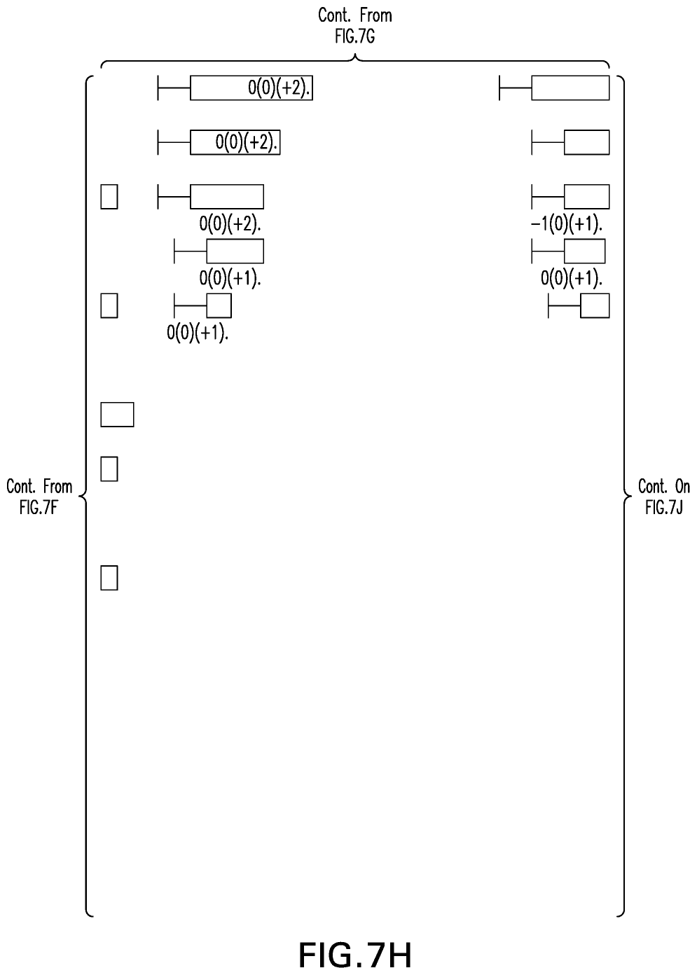

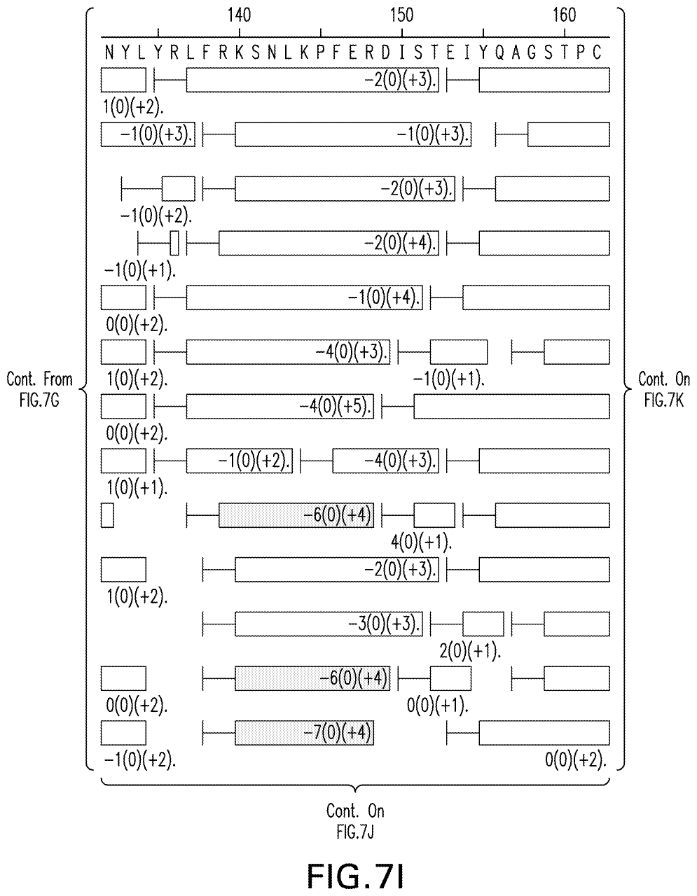

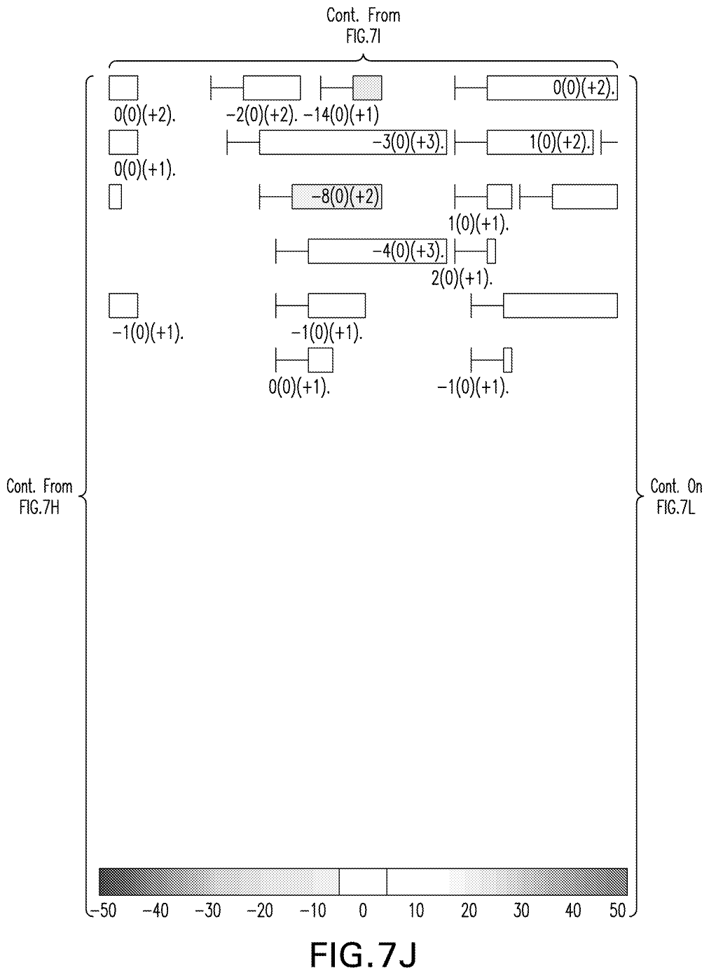

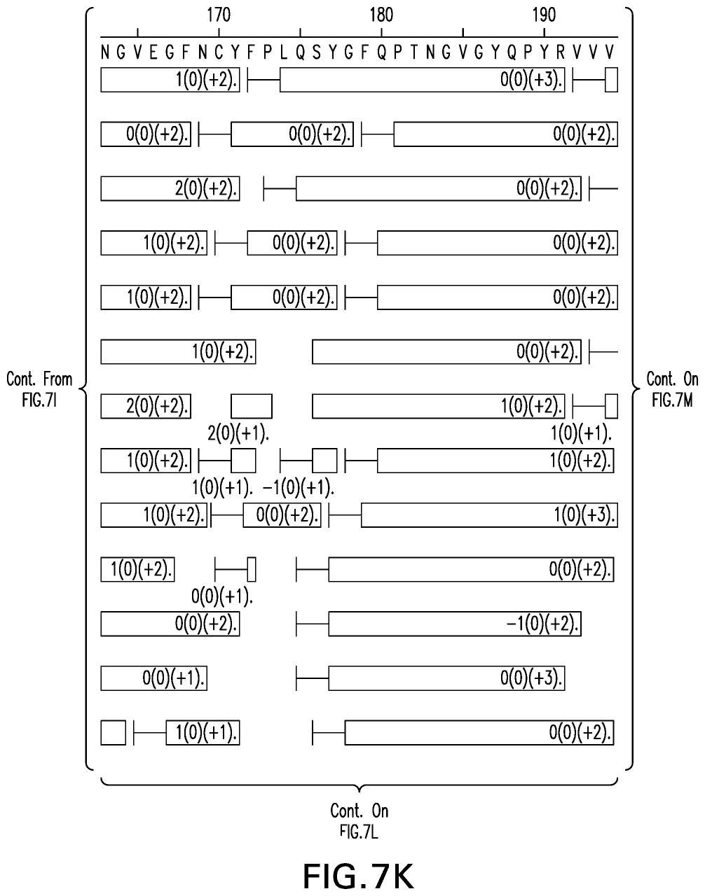

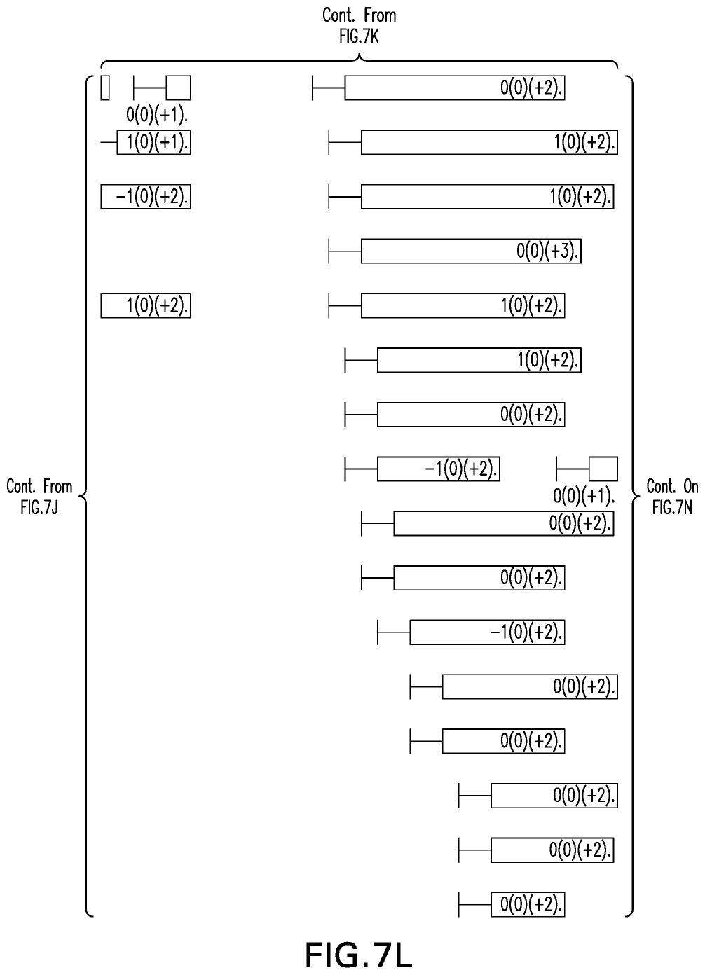

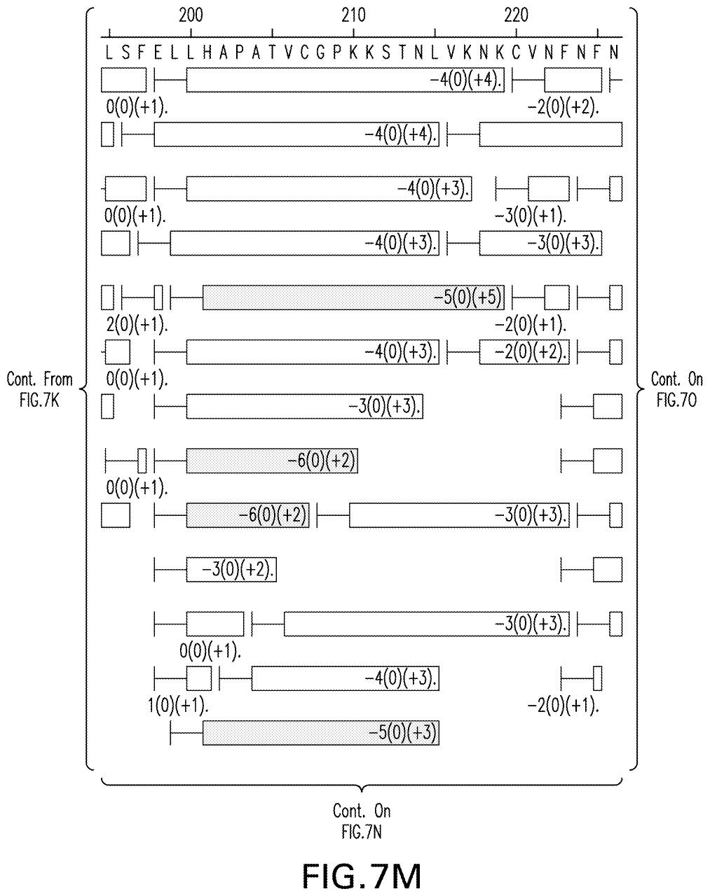

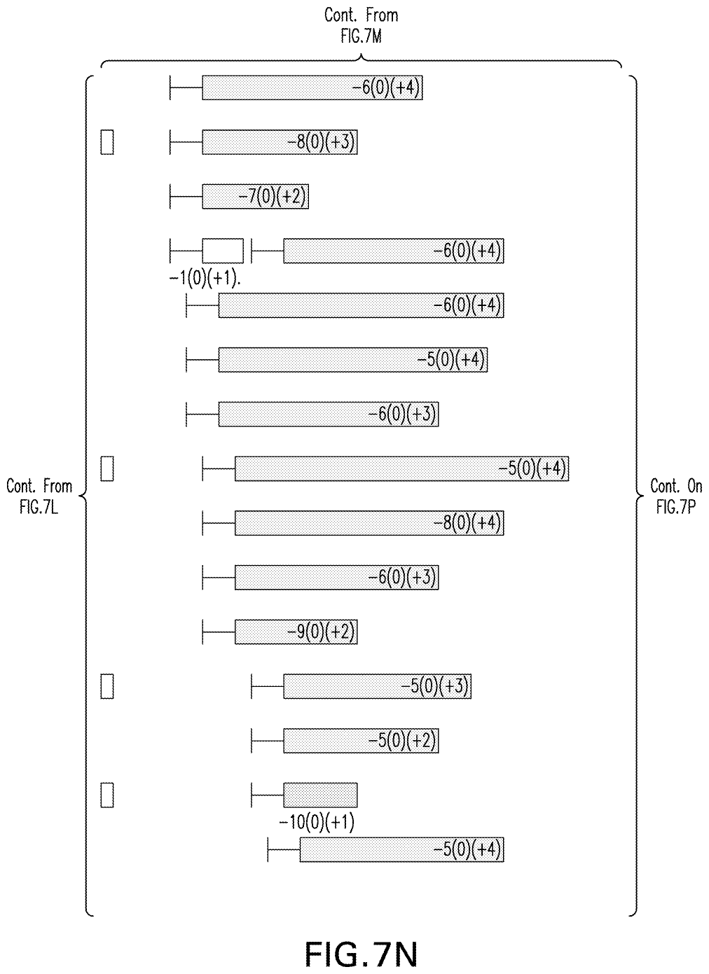

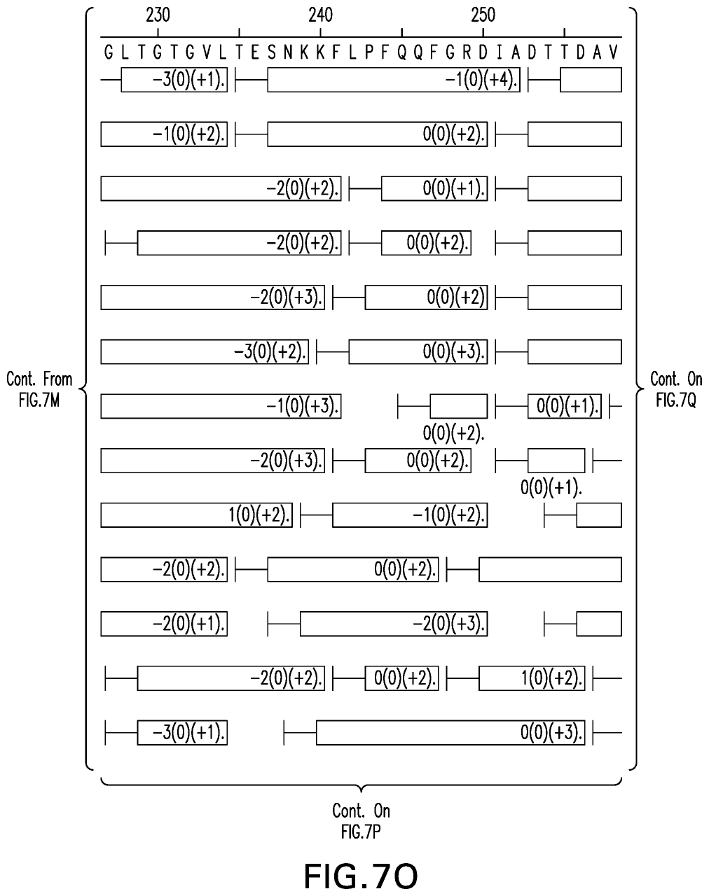

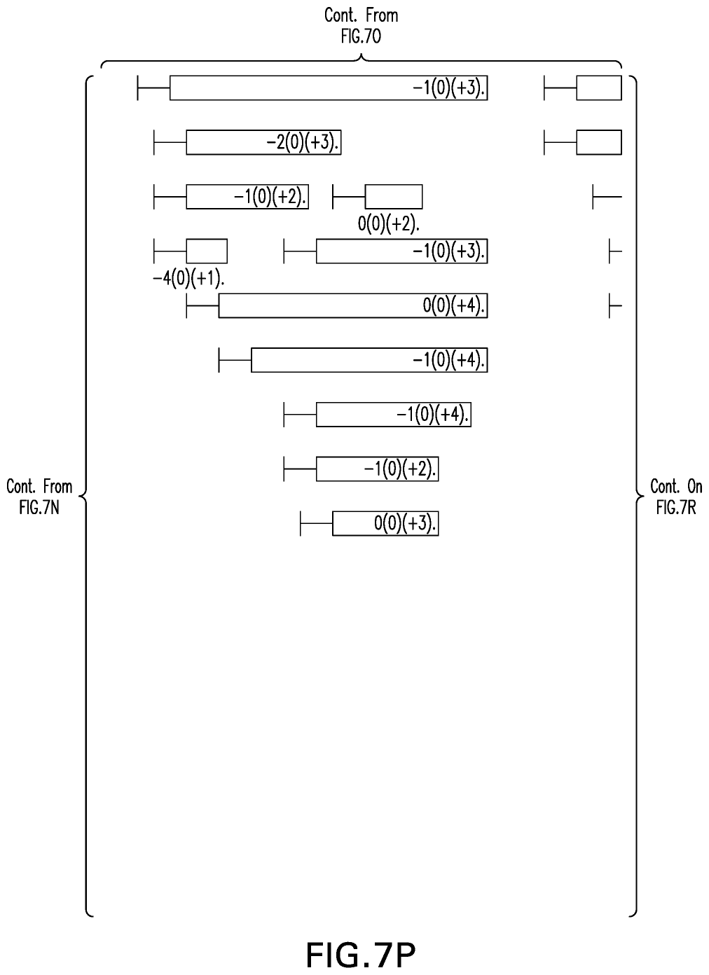

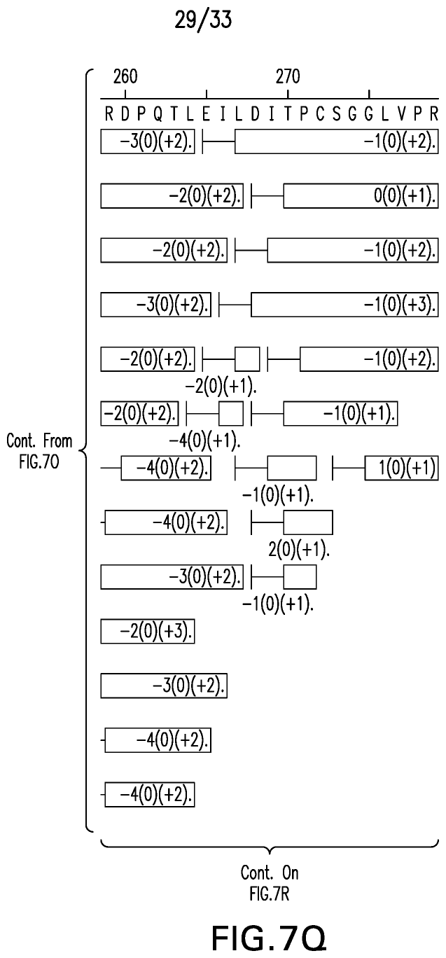

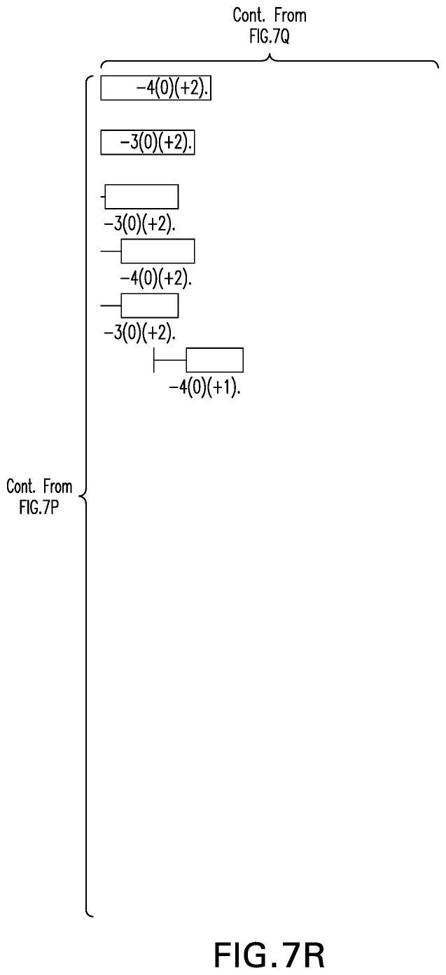

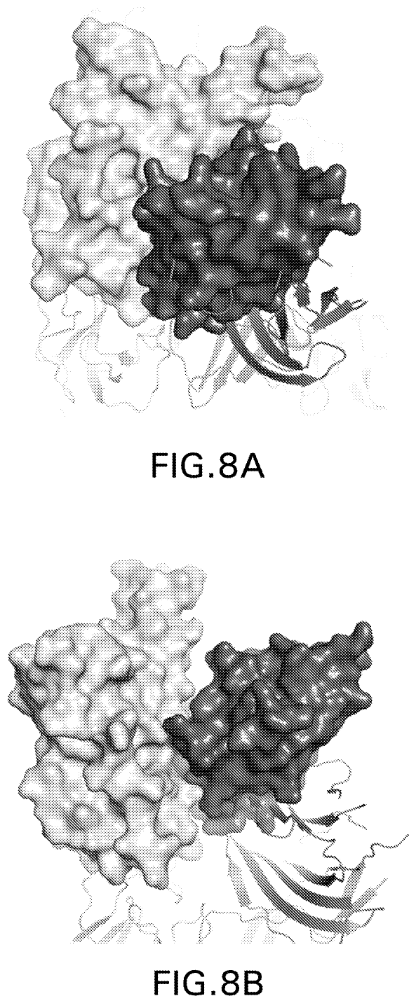

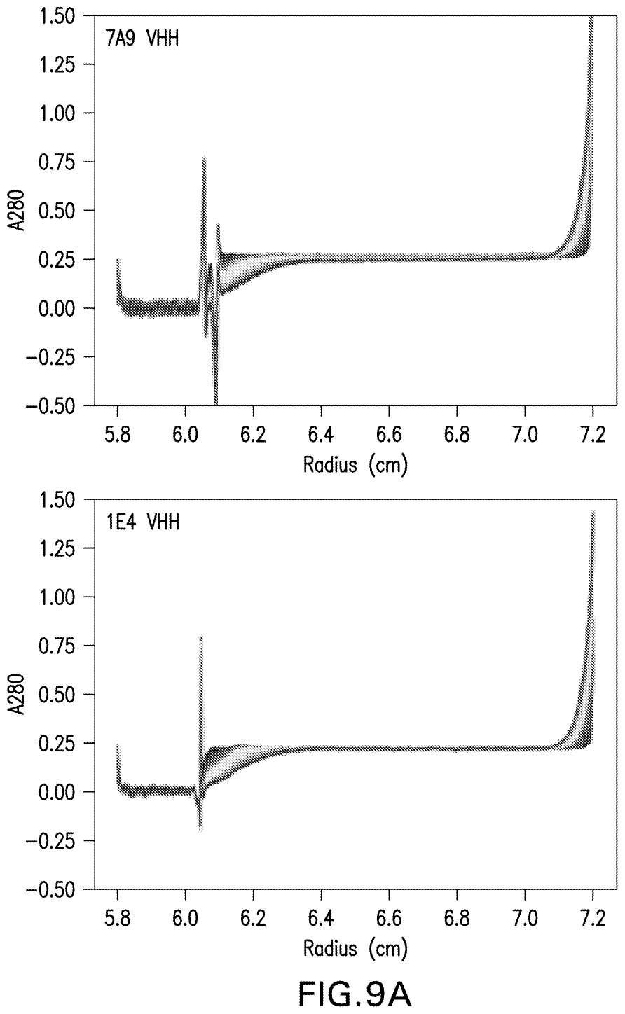

We were intrigued that the RBD-binding VHH 7A9 bound to a cross-reactive epitope that did not bin with any of the benchmark antibodies used in this study. To characterize the 7A9 epitope we used HDX-MS, which indicated that the major binding site of 7A9 is partially occluded by the NTD and spans residues 353-364 of the spike protein (FIGS. 7A-7R). To further analyze this rare antigenic site, we used X-ray crystallography to determine the structure of 7A9 bound to the CoV-2 RBD (FIG. 4A to FIG. 4E). The crystal structure reveals that the third complementarity-determining region (CDR3) of 7A9 forms a platform through which the VHH binds to a concave cleft on the RBD, inserting Leu107 into a small cleft formed by Tyr396 and Phe464. CDR1 and CDR2 contribute only minimally to the interaction, with Arg31 of CDR1 forming a network of Van der Waals interactions and Tyr60 of CDR2 forming a hydrogen bond with Glu465. The energetic driver of the interaction is likely the salt bridges formed between Arg357 of the RBD and two 7A9 glutamates in CDR3, Glu104 and Glu1 19. A detailed view of the molecular interactions is shown in FIG. 4B. The structure helps to rationalize how 7A9 is still able to bind the Omicron variant, as the epitope does not overlap with any of the 15 RBD mutations in this VOC (FIG. 4C). Notably, the 7A9 epitope is fully occluded in the closed state and partially occluded in the open state of the spike protein (FIG. 4D). Modeling 7A9 binding onto the full CoV-2 spike protein indicates that the VHH would introduce a clash with the NTD from the neighboring spike protomer in either the closed- or open state (FIG. 8A and FIG. 8B). This suggests that either a conformational shift in the RBD relative to the NTD or trimer dissociation would have to occur upon VHH binding.

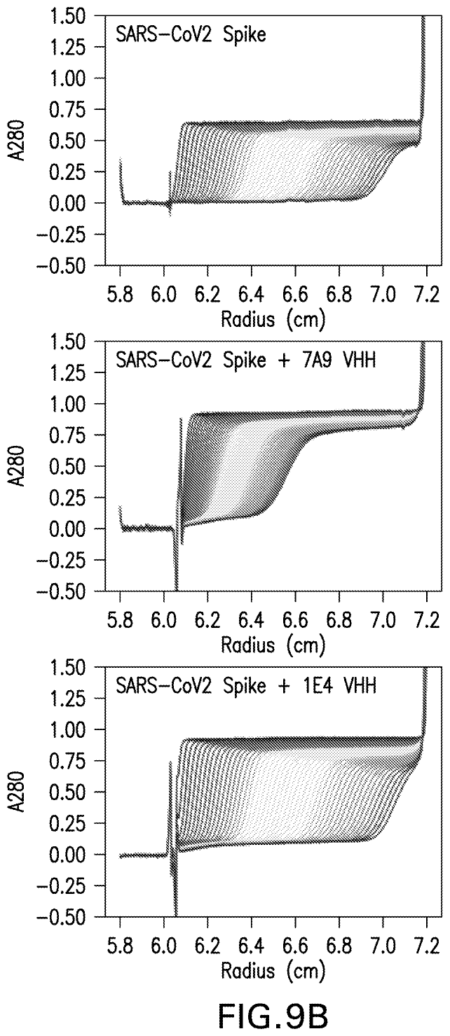

To understand the structural consequences of 7A9 binding to the SARS-CoV2 spike trimer, we conducted a series of analytical ultracentrifugation sedimentation velocity (AUC-SV) experiments, using a continuous distribution (c(s)) analysis to determine the species present in solution (FIG. 4E). The c(s) distribution of the spike alone showed as a single peak (95% of total signal) at 15.9s, corresponding to an apparent mass of 441 kDa, consistent with a stable trimer (estimated mass of 398 kDa). 7A9 VHH showed as a single peak (93% of total signal) at 2.1s, corresponding to an apparent mass of 18 kDa, as expected. 7A9 was then mixed with the spike trimer at a 1.5:1 (monomer: monomer) molar ratio. Compared to the spike trimer alone, this mixture displayed a dramatic shift in the sedimentation profile that is readily apparent from the absorbance scans (FIG. 9A and FIG. 9B). The c(s) distribution revealed 2 major species at 2.2s (7%) and 8.0s (86%), with apparent masses of 22 and 155 kDa, respectively. We interpret these species as excess free 7A9 VHH and 7A9 bound to a spike monomer (theoretical mass of 151 kDa). Strikingly, there was no appreciable intact spike trimer (15.9s) remaining after mixing with 7A9, indicating that binding of 7A9 completely dissociated the spike trimer. To determine if spike trimer dissociation was specifically due to 7A9 binding, we ran identical AUC-SV experiments using the 1E4 VHH, which is predicted to bind to, but not disrupt, the spike trimer. Similar to 7A9 alone, the c(s) distribution of 1E4 alone showed a single peak (98% of total signal) at 2.1s, corresponding to an apparent mass of 19 kDa. Mixing 1E4 with the spike trimer at a 1.5:1 (monomer: monomer) ratio showed 2 major species at 2.0s (8%) and 16.7s (88%), with apparent masses of 20 and 467 kDa, respectively. We interpret these species as excess free 1E4 VHH and 1E4 bound to the spike timer (theoretical mass of 451 kDa). Together, these data clearly indicate that the binding of 7A9 to its cryptic epitope results in the complete dissociation of the SARS-CoV2 spike trimer, whereas the spike trimer remains intact upon 1E4 binding.

### Multimeric Design Enhances Neutralization

It has been reported that multimerization of VHHs can lead to improvement in potency. (Schoof et al. 2020, 370:1473-1479, Xu et al. 2021, 595:278-282; Xiang et al. 2020, Science 370:1479). Given both the size of the spike protein as well as its trimeric nature, we employed two approaches to achieve higher potency and potentially protect against escape mutations. The first approach was to design homotrimers of RBD-binding VHHs (1E4 and 10B8) to engage each monomer of the spike protein simultaneously and improve potency. Alternatively, we also designed heterotrimers targeting three different sites on the spike protein, which would in theory be more resistant to escape mutants, as the loss of affinity at any one site could be compensated for by the binding at the other sites. A number of homo- and hetero-trimeric constructs were designed using the results of domain binning, HDX mapping, and modeling to determine the optimal lengths of the linkers between each VHH (see Example 2).

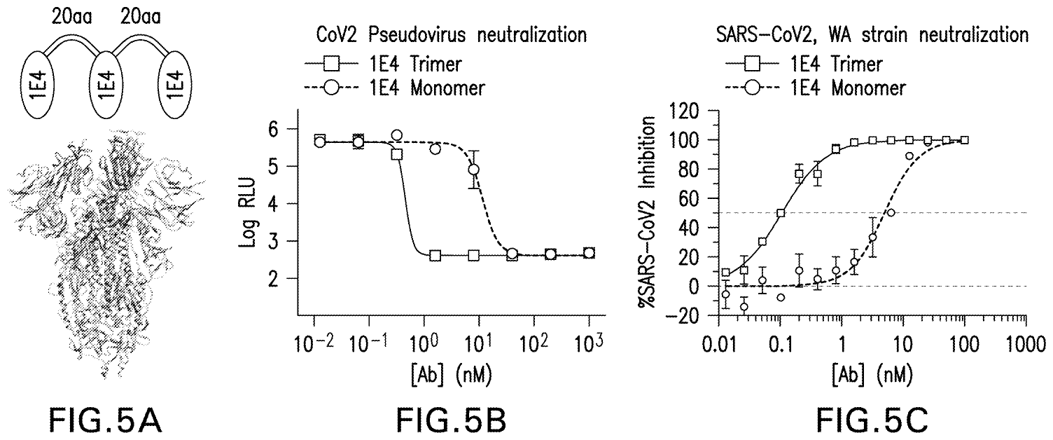

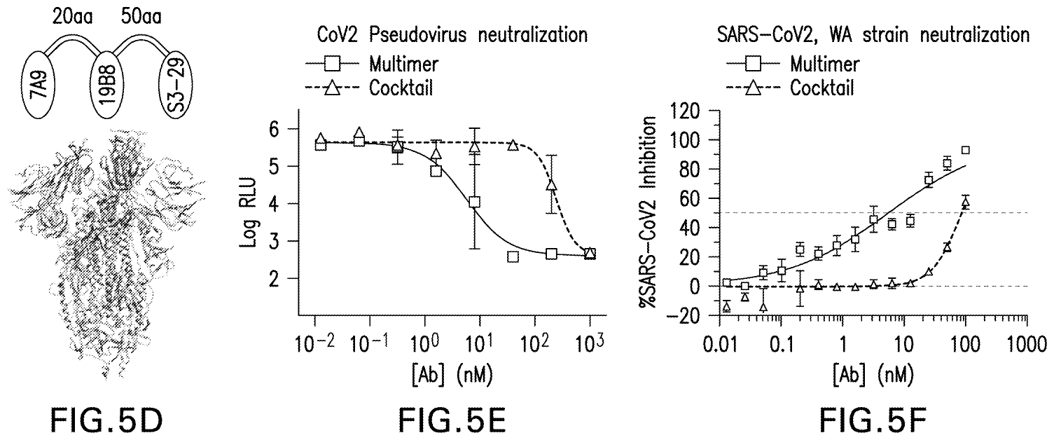

To determine whether multimerization enhances potency, we compared neutralization potency of multimeric constructs with cocktail mixtures of individual VHHs used in the respective multimer (FIG. 5A to FIG. 5F, Table 6). A homotrimer of 1E4 showed about a 26-fold improvement in neutralization SARS-CoV2 pseudovirus neutralization and about 46-fold improvement in neutralization for SARS-CoV2 authentic virus (FIG. 5A to FIG. 5C). Likewise, a heterotrimeric construct targeting RBD, NTD and S2 showed a 43-fold improvement in neutralization over a cocktail of three VHHs in SARS-CoV2 pseudovirus assay (FIG. 5D, FIG. 5E). Importantly, this potency improvement was also observed in authentic SARS-CoV2 neutralization (FIG. 5F). We observed multimerization leading to improved potency with several designs where weakly-neutralizing or non-neutralizing VHHs could be combined to enhance potency (Table 6). Pseudovirus neutralization potencies of multimers against SARS-CoV2 and Omicron variant were also determined (Table 7).

TABLE 6
Pseudovirus neutralization potency of multimer vs cocktail

| | | Multimer | Cocktail | | Multimer | Cocktail | |
|---|---|---|---|---|---|---|---|
| | | CoV2 | CoV2 | | SARS | SARS | |
| | | IC50 | IC50 | Fold | IC50 | IC50 | Fold |
| Clone | Epitopes | (nM) | (nM) | improvement | (nM) | (nM) | improvement |
| 7A9-10B8-19B8 | RBD1-RBD2-NTD | 2.79 | 0.75 | 0.3 | N/A | N/A | N/A |
| 10B8-19B8-20D11 | RBD2-NTD-S2-2 | 1.04 | 2.08 | 2 | N/A | N/A | N/A |
| 7A9-1E4-6A1 | RBD1-RBD2-S2-1 | 1.99 | 3.72 | 1.9 | N/A | N/A | N/A |
| 1E4-7A9-6A1 | RBD2-RBD1-S2-1 | 2.88 | 3.72 | 1.3 | N/A | N/A | N/A |
| 10B8-7A9-S3_29 | RBD2-RBD1-S2-3 | 0.99 | 5.9 | 6 | N/A | N/A | N/A |
| 10B8-19B8-S3_29 | RBD2-NTD-S2-3 | 0.31 | 1.97 | 6.4 | N/A | N/A | N/A |
| 10B8-7A9-19B8 | RBD2-RBD1-NTD | 1.7 | 2.6 | 1.5 | N/A | N/A | N/A |
| 10B8-19B8-6A1 | RBD2-NTD-S2-1 | 1.58 | 2.75 | 1.7 | N/A | N/A | N/A |
| 7A9-10B8-S3_29 | RBD1-RBD2-S2-3 | 2.5 | 5.9 | 2.4 | 0.52 | 0.19 | 0.37 |
| 7A9-19B8-S3_29 | RBD1-NTD-S2-3 | 5.7 | 246 | 43 | 1.3 | 344 | 265 |
| 7A9-19B8-20D11 | RBD1-NTD-S2-2 | 29 | 136 | 4.7 | 11 | 947 | 86 |
| 7A9-19B8-6A1 | RBD1-NTD-S2-1 | 69 | 233 | 3.4 | 6.81 | 1205 | 177 |
| 19B8-S3_29-7A9 | NTD-S2-3-RBD1 | 1.14 | 87 | 76 | 6.09 | 431 | 71 |
| 19B8-S3_29-S3_29 | NTD-S2-3-S2-3 | 15 | 436 | 29 | 95 | 1156 | 12 |
| S3_29-S3_29-7A9 | S2-3-S2-3-RBD1 | 53 | 162 | 3 | 104 | 1092 | 10 |
| S3_29-19B8-6A1 | S2-3-NTD-S2-1 | 20 | 763 | 39 | 16 | 1197 | 73 |
| S3_29-S3_29-6A1 | S2-3-S2-3-S2-1 | N/A | N/A | N/A | N/A | N/A | N/A |
| S3_29-11F5-6A1 | S2-3-S2-2-S2-1 | N/A | N/A | N/A | N/A | N/A | N/A |
| 10B8-19B8-StdE | RBD2-NTD-HIV | <1 | 1.13* | — | <1 | 0.34* | — |
| 10B8-7A9-StdE | RBD2-RBD1-HIV | <1 | 1.42 | — | <1 | 0.10 | — |
| 7A9-19B8-90aa-StdE | RBD1-NTD-HIV | 77 | 965 | 12 | 8.2 | 1300 | 159 |
| 7A9-19B8-50aa-StdE | RBD1-NTD-HIV | 2780 | 965 | 0.35 | 105 | 1300 | 12.4 |
| 19E10-S3_29-6A1 | RBD2-S2-3-S2-1 | 0.19 | 32 | 170 | 21 | 253 | 12 |
| 19E10-S3_29-7A9 | RBD2-S2-3-RBD1 | <1 | 31 | — | 1.77 | 335 | 189 |
| 19E10-19B8-6A1 | RBD2-NTD-S2-1 | 5.9 | 27 | 4.6 | 0.65 | 298 | 456 |
| 19B8-S3_29-19E10 | NTD-S2-3-RBD2 | 0.05 | 32* | 640 | <1 | 239* | — |
| 19E10-11F5-6A1 | RBD2-S2-2-S2-1 | 3.2 | 37 | 12 | 259 | 272 | 1.05 |
| 19E10-S3_29-19E10 | RBD2-S2-3-RBD2 | <1 | 36 | — | 0.38 | 202 | 531 |

TABLE 7
Pseudovirus neutralization potency of selected
multimers against SARS-Cov2 and Omicron variant

| | | CoV2 | Omicron |
|---|---|---|---|
| | | WT IC50 | IC50 |
| Clone Name | Epitopes | (nM) | (nM) |
| 10B8-7A9-S3_29 | RBD2-RBD1-S2-3 | 0.24 | 2.8 |
| 10B8-19B8-S3_29 | RBD2-NTD-S2-3 | 0.20 | 1.8 |
| 10B8-7A9-19B8 | RBD2-RBD1-NTD | 0.31 | 3.1 |
| 7A9-19B8-S3_29 | RBD1-NTD-S2-3 | 4.1 | 23 |
| 10B8-7A9-StdE | RBD2-RBD1-HIV | 0.41 | 5.0 |
| 19E10-S3_29-6A1 | RBD2-S2-3-S2-1 | 0.74 | 128 |
| 19E10-S3_29-7A9 | RBD2-S2-3-RBD1 | 0.50 | 45 |
| 19E10-19B8-6A1 | RBD2-NTD-S2-1 | 3.2 | 78 |
| 19B8-S3_29-19E10 | NTD-S2-3-RBD2 | 0.21 | 45 |
| 19E10-S3_29-19E10 | RBD2-S2-3-RBD2 | 0.11 | 3.0 |
| 10B8 Trimer | RBD2 | 0.44 | 0.46 |

S3-29 epitope: In the closed state of the Spike protein, this region is not exposed or accessible to VHHs. This region could be exposed for VHH binding in one of two ways; (a) if multiple RBDs were in the open state, or (b) following shedding of S1 but prior to the conformational change that precedes membrane fusion. Intriguingly, there is a previous report of an antibody that targets the same region that was mapped similarly using HDX (Huang et al. 2021 bioRxiv 2021.01.31.428824), providing further evidence that this site is accessible.

6A1 epitope: While not resolved in most spike protein structures, previous studies have found similar antibodies (Wang et al. 2021 Nature Comm 12:1715, Sauer 2021 Nature Struc & Mol Biol 28:478, Li et al. 2022 Cell Reports 38:110210, Pinto et al. 2021 Science 373:1109). This region is exposed regardless of the conformational state of the prefusion Spike protein.

11F5 epitope: This antibody appears to cause both protection and deprotection effects upon binding to the bottom of the spike, suggesting that spike undergoes a local conformational change when this antibody binds.

Multimers: Most of the multimeric VHHs that showed highest fold increase in neutralization potency appear to include the S3-29 component. While not wishing to be bound by theory, we speculate that this epitope, which is located relatively closer to the S1 domain of spike, might help to stabilize the RBD or NTD targeting components to bind their epitopes.

### Example 2: Materials and Methods

### Plasmid Construction, Protein Expression and Protein Purification

### Spike Protein

The SARS-CoV prefusion-stabilized spike protein (SARS-CoV-PreS) includes the SARS-CoV spike protein ectodomain residues 1-1190 (amino acid 1 denotes the starting methionine in the signal peptide) two proline substitutions at K968P and V969P, a C-terminal T4 fibritin trimerization domain, an HRV3C protease cleavage site, an 8x His-tag (SEQ ID NO: 251) as described previously. (Kirchdoerfer et al. 2018 8:15701) The SARS-CoV spike ectodomain protein where the His-tag was not cleaved (CoV-PreS-3C) contained a thrombin cleavage site in place of the HRV3C protease site. The MERS prefusion-stabilized spike protein (MERS-PreS) includes the MERS spike protein ectodomain residues 1-1291, two proline substitutions at V1060P and L1061P, an “ASVG” (SEQ ID NO: 252) substitution at the furin cleavage site (residues 748-751, RSVR (SEQ ID NO: 253)), a C-terminal T4 fibritin trimerization domain, a thrombin cleavage site, and an 8x His-tag (SEQ ID NO: 251) similar to previously described. (Pallesen et al. 2017, 114: E7348-E7357) The SARS-CoV-2 prefusion-stabilized spike protein (SARS-CoV-2-PreS) includes SARS-CoV-2 spike protein ectodomain residues 1-1208 two proline substitutions at K986P and V987P, a “GSAS” (SEQ ID NO: 254) substitution at the furin cleavage site (residues 682-685, RRAR (SEQ ID NO: 255)), a C-terminal T4 fibritin trimerization domain, a thrombin cleavage site, and an 8x His-tag (SEQ ID NO: 251) similar to previously described. (Wrapp et al. 2020 367:1260-1263) The “closed” conformation SARS-CoV-2 spike protein trimer (CoV-2-PreS-Closed) contains the SARS-CoV-2 spike protein ectodomain residues 1-1208, four amino acid substitutions (D614N, A892P, A942P, and V987P), a thrombin cleavage site, and an 8x His-tag (SEQ ID NO: 251) similar to previously described (Juraszek et al. 2021, 12:244). The Receptor Binding Domain (RBD) and N-Terminal Domain (NTD) only protein constructs were designed as follows. The CoV-2-S-RBD-SDI spike and CoV-2-S-NTD Spike constructs contain residues 319-591 and 1-305 respectively cloned in frame with the native CoV-2 signal sequence and appended with a C-terminal T4 fibritin trimerization domain, a thrombin cleavage site, and an 8x His-tag (SEQ ID NO: 251) similar to previously described (Wrapp et al. 2020, 367:1260-1263). The CoV-S-RBD-SD1 and MERS-S-RBD-SD1 spike protein constructs contain residues 306-577 and 367-655 respectively cloned in frame with the native protein's signal peptide, a thrombin cleavage site, and an 8x His-tag (SEQ ID NO: 251). The S2 domain only SARS-CoV-2 spike protein (CoV-2-PreS-S2) construct includes ectodomain residues 697-1208 with two proline substitutions at K968 and V969 cloned in frame immediately downstream of an IgK signal peptide and appended with a T4 fibritin trimerization domain, thrombin cleavage site and 8x His-tag (SEQ ID NO: 251) at the C-terminus. Biotinylated constructs include an Avi-tag sequence immediately upstream of the protease cleavage site flanked by GRS-GG linkers. The CoV-2-S-RBD construct used for x-ray crystallography contains residues 319-541 and was cloned in frame with the native CoV-2 signal sequence and contained a C-terminal SG-6×His. All gene-encoding regions were mammalian codon-optimized and subcloned into a eukaryotic-expression vector under the control of the CMV promotor.

Plasmids were transiently transfected into Expi293F™ cells (ThermoFisher) using Expifectamine™ (ThermoFisher) following manufacturer's recommended protocol. Cell supernatants were harvested 72 hours post-transfection and clarified by centrifugation at 10,700×g at 20° C. for 30 minutes. Polysorbate (PS)-20 was added at a final concentration of 0.01% to the clarified supernatants to mitigate aggregation. Clarified supernatants were aliquoted into 250 mL Corning bottles and transferred to −70° C. for storage until purification.

Clarified supernatant was thawed in a 37° C. shaking water bath and carried forward into purification. Spike protein was purified from cell culture supernatant using immobilized metal affinity chromatography (IMAC) on a HisTrap™ Ni Sepharose column (GE Healthcare) and eluted with 300 mM imidazole. The His-tag was removed by overnight digestion at room temperature with either thrombin or HRV3C protease as appropriate. Spike protein was further purified by a second, subtractive affinity chromatography step (IMAC2) to remove protease, IMAC contaminants, cleaved His-tags and uncleaved Spike. Final purification of the sample was achieved with size exclusion chromatography (SEC) using a Superdex™ 200 column (GE Healthcare) for SARS-CoV2-PreS and MERS-PreS and using a Sephacryl™ S-300 column (GE Healthcare) for SARS-CoV-PreS. Prominent protein containing fractions were pooled, concentrated, and dialyzed into 50 mM 4-(2-hydroxyethyl)-1-piperazineethanesulfonic acid (HEPES), pH 7.5 and 300 mM NaCl. Final protein concentration was determined by A280 analysis using protein-specific reduced extinction coefficients calculated from amino acid sequence. Protein purity was estimated by Coomassie-stained gel. Protein oligomerization state and size were confirmed using size-exclusion chromatography combined with multiangle laser light scattering (SEC-MALLS).

For biotinylated SARS-CoV-2 ectodomains, purification was completed as described. Following SEC, protein was biotinylated using BirA Ligase (Avidity LLC) at a 1:400 BirA: Protein ratio. Protein was further purified and BirA Ligase was removed by a second SEC step (SEC2).

For biotinylated CoV-2-S-RBD-SD1 and CoV-2-S-NTD proteins, purification included IMAC1 and SEC1. Protein was biotinylated using BirA ligase (Avidity LLC) at a 1:60 BirA: Protein ratio. Spike protein was further purified by a second, subtractive affinity chromatography step (IMAC2) to remove BirA ligase. The His-tag was removed by overnight digestion at room temperature with CleanCleave™ immobilized thrombin resin (Sigma-Aldrich) followed by centrifugation to remove the resin and a third IMAC step (IMAC3) to remove uncleaved protein and cleaved His-tags. Final purification of the sample was achieved with a second size exclusion chromatography (SEC2) step using a Superdex™ 200 column (GE Healthcare).

Construct design, expression and purification of counterscreen protein human MPV post-fusion F trimer has been published previously (Xiao et al. 2022 13:2546).

For the x-ray structure sample, CoV-2-S-RBD (519-541) was harvested 72 hours post-transfection, supernatant was clarified by centrifugation, and concentrated/buffer exchanged by tangential flow filtration into 25 mM HEPES pH 7.5, 300 mM NaCl. Sample was purified by IMAC over a HisTRAP™ FF column (Cytiva) and subsequent Superdex™ 75 column in 25 mM HEPES pH 7.5, 150 mM NaCl. Sample was concentrated to 10 mg/mL and flash frozen. The 7A9 VHH was harvested 6 days post-transfection and clarified. Supernatant was purified by IMAC over a HisTRAP™ Excel column (Cytiva) in 25 mM HEPES pH 7.5, 300 mM NaCl and subsequent Superdex™ 75 in 25 mM HEPES pH 7.5, 150 mM NaCl. Sample was concentrated to 10 mg/mL and flash frozen. For the RBD/7A9 VHH complex, samples were thawed and mixed using excess VHH 1:1.5 (RBD: VHH) and incubated at 4C for one hour. Complex was then purified over a Superdex™ 75 column in 25 mM HEPES pH 7.5, 150 mM NaCl. Final purified complex was concentrated to 10.7 mg/mL and flash frozen.

Recombinant SARS-CoV-2 spike proteins from different variants used in ELISA were obtained from Acro Biosystems-B.1.1.7 variant (SPN-C52H6), B.1.351 variant (SPN-C52Hk), P.1 variant (SPN-C52Hg), B.1.617.2 variant (SPN-C52He), BA.2 (SPN-C5223), BA1.1 (SPN-C52 Hz).

### VHH-Fc

VHH-Fc, VHH monomer, and VHH multimer (“beads-on-a-string”) antibody chains were cloned into our in-house pTT5 based vector carrying Lonza leader secretion tags and CMV promoter. VHH-Fc and VHH monomer plasmids were transiently transfected into ExpiCHO™ expression system using serum-free defined media, and suspension adapted CHO cells following manufacturer recommendations with Max-Titer protocol. Cells were harvested after 7-days with feeds on day 1 and 5 and temperature shift to 32C on Day1. High-throughput (HT) Protein A (Mabselect) affinity chromatography in miniature columns (Robocolumns) was used for capture and enrichment of recombinant antibodies from clarified harvest cell culture fluid (HCCF). Analytical size-exclusion (aSEC) to characterize solution behavior and capillary electrophoresis (CE-SDS) to characterize under denaturating conditions was used to QC the expressed molecules. An acceptable criteria of overall purity was chosen for selected molecules to be used in downstream assays.

### VHH-Multimer

VHH multimer plasmids were transiently transfected into Expi293™ expression system in suspension using serum-free defined media following manufacturer recommendations. Cell culture supernatants were harvested after 5-days with feed on day 1. Ni affinity (Ni Sepharose Excel-Cytiva) chromatography using gravity flow columns enabled 1-step purification of recombinant antibodies from clarified harvest cell culture fluid. aSEC was used to characterize the multimers.

### Llama Immunization

Four llamas were immunized at Capralogics using a heterologous recombinant protein immunization boost strategy. This approach involved an initial subcutaneous injection of all four llamas with 0.5 mg of SARS CoV2 spike protein mixed with Complete Freund's adjuvant. For the remaining two injections, two llamas incorporated incomplete Freund's adjuvant (IFA) and two llamas used no additional adjuvant. The injections were all three weeks apart and included a second subcutaneous injection of 0.5 mg of MERS protein and then a third subcutaneous injection of 0.5 mg of SARS CoV1 spike protein. Ten days after the final injection, 200 mL of whole blood was collected from the llamas in EDTA blood collection tubes. PBMCs were then isolated from the blood using density gradient centrifugation. Serum titers were also evaluated against SARS CoV2, SARS CoV1, and MERS spike protein by ELISA and two llamas were selected for further processing (one that used an IFA injection strategy and one that used no adjuvant for injections other than the primary injection.)

### Llama B-Cell Sorting, Culture, and Sequencing

Purified llama PBMCs from the immunized animals were cell surface stained using biotinylated SARS CoV2 or biotinylated MERS spike protein, goat anti-llama IgG (H+L) FITC conjugate (Thermo cat #A16061), 7-AAD live/dead cell stain (Biolegend, 420404), and streptavidin BV421 (Biolegend cat #405226). Live B-cells, positive for biotinylated antigen and IgG, were sorted as single cells into 96-well plates or sorted in bulk into tubes using a BD FACSAria Fusion.

The single sorted cells were cultured for two weeks with a gamma irradiated CD40L-EL4 recombinant cell line and internally made llama IL-2/IL-21 cytokines at 37C. After two weeks, B-cell culture supernatants were screened using ELISA and FACS for binding to recombinant or cell expressed proteins. Candidates of interest were then lysed in 85 μL of Qiagen TCL buffer with 1% B-mercaptoethanol and RNA was isolated using Qiagen Turbocapture™ tubes (Qiagen cat #72251).

cDNA was generated using SuperScript™ IV reverse transcriptase (Thermo, cat #18090050) in the presence of a template switching oligo (TSO) for 5′RACE. A first round PCR reaction was then performed using GoTaq® polymerase (Promega, cat #M7422), a llama heavy chain only gene specific constant region reverse primer, and a TSO-compatible forward primer. A second round of PCR, also using GoTaq® polymerase, further amplified the PCR products and introduced Illumina MiSeq™ NGS adaptors and indices for high-throughput, multiplexed NGS sequencing. Individual, indexed PCR products were then pooled and gel purified using a Qiagen gel purification kit (Qiagen cat #28704.) Sequences that showed cross-reactivity between 2 or more strains by ELISA or FACS were identified and recombinantly expressed for further testing.

The bulk sorted B-cells were immediately lysed in Qiagen buffer RLT with 1% B-mercaptoethanol, and RNA was isolated using the Qiagen RNeasy® micro kit (cat #74004.) cDNA was generated using Superscript™ IV reverse transcriptase (Thermo, cat #18090050) in the presence of a template switching oligo (TSO) containing a unique molecular identifier (UMI) to enable 5′RACE and error correction during analysis of the NGS sequencing data. Two rounds of PCR were then performed using KAPA HiFi Polymerase (KAPA, cat #KK2501), a llama heavy chain only gene specific constant region reverse primer, and a TSO-compatible forward primer. The primers also included indices to identify specific samples and Illumina MiSeq™ adaptors. Individual, indexed PCR products were then pooled and gel purified using a Qiagen gel purification kit (Qiagen cat #28704.) Bioinformatic analysis was used to compare sequencing data from B-cells sorted using SARS CoV2 spike protein and MERS spike protein to look for sequences in common between the two libraries, indicating potential cross-reactivity of a sequence for at least two strains. Two candidates identified using this approach were recombinantly expressed.

### Protein ELISA

96-well half area plates were coated with either 25 μL/well of full-length spike proteins or domain proteins (1 μg/mL in PBS buffer) and incubated overnight at 4° C. The next day, plates were washed 3 times with PBST (PBS+0.05% Tween 20) and blocked with 25 μL/well of blocking buffer (PBS with 5% FBS) for 30 minutes at room temperature. B-cell culture supernatant or titrated purified antibody was then transferred at 25 μl/well to the 96-well plates and incubated for 60 minutes at room temperature. The plates were then washed 3 times with PBST. Then 25 μl/well of rabbit anti-llama IgG (H+L) HRP (1:1000 dilution in blocking buffer, Thermo Scientific cat #A16154) or a secondary antibody specific for the Fc of the purified antibody was added to the plates and incubated for 60 minutes at room temperature. Finally, the plates were washed 5 times with PBST and developed by adding TMB reagent (Thermo cat #34029) to the plates for 2-3 minutes. The reactions were stopped with 0.16M sulfuric acid and the absorbance read at 450 nm and 650 nm using a spectrophotometer.

### Multiplex FACS Cell Binding

Recombinant CHO K1 cells lines expressing spike proteins from different virus strains (CoV1, CoV2, MERS, HKU1 and OC43) were harvested using trypsin and washed twice with PBS buffer. Cell trace dye (CellTrace-violet Thermo Scientific cat #C34557 and CellTrace-Far Red Thermo Scientific cat #34564) was diluted at optimized concentrations in PBS (1 ml staining volume/10M cells) and used to resuspend cell pellets prepared from the different recombinant cell lines. The cells were incubated with the dyes at 37° C. for 30 minutes in the dark, with occasional swirling. These staining reactions were stopped by adding warm DMEM/F12 complete medium with 10% FBS, using 5× the original staining volume, and incubated at 37° C. for 10 minutes. Stained cells were spun down and washed once with 10 ml PBS and resuspended in FACS buffer (5% fetal bovine serum in PBS.) Cells were then checked to confirm both positive staining with the dyes and that each recombinant cell line demonstrated a separate fluorescent intensity using an Intellicyt®. The different recombinant cell lines were then mixed by resuspending in FACS buffer and aliquoted into 96 well plates (50 μl/well, 80×10⁴/well). Cells were stained with either 50 μl of titrated, purified antibodies or B-cell culture supernatant for 30 minutes and then spun down and washed 1× with FACS buffer. Finally, the cells were stained with a fluorescently labeled secondary antibody (specific to the Fc domain of the supernatant or recombinant antibody) for 30 minutes and spun down and washed 2× with FACS buffer. Cells resuspended in 50 μl FACS buffer were then analyzed using an Intellicyt®.

### Domain Binning

Tandem binning experiments were performed on Octet® HTX using BLI technology to bin anti-Cov2-spike protein antibodies. Biotinylated CoV2 Spike ECD PreS-AVI protein (20 nM) was captured on a streptavidin (SA) coated biosensors (Sartorius) for 20 minutes to get 1.5-2 nm binding response. First antibody (20-40 μg/mL) was flowed over to the trimer Cov2 spike protein until all the binding sites get saturated, followed by the binding of 20-40 g/mL of 2^{nd} antibody. Two benchmarks, Std-B (class 1 binder) & Std-A (class 3 binder) and Std-D (Class 4 binder) were used to classify RBD domain binder. Four in-house generated antibodies were used to screen NTD and S2 domain binders. Kinetic buffer (1XKB, PBS+0.02% Tween20, 0.1% BSA, 0.05% sodium azide) was used as a running buffer. Biosensors were regenerated (1.7 nM Glycine) followed by a buffer wash twice after each cycle. Black 384 well polypropylene tilted bottom plates were used for binning experiment.

### Pseudovirus Neutralization

### Generation of Recombinant VSVAG-Based Pseudoviruses Carrying Firefly Luciferase (Luc) Reporter Gene and Coronavirus Spike(s) Proteins

Pseudovirus particles were made as previously described by Whitt and Schmidt et al. Briefly, to generate rVSVAG-Luc pseudoviruses, 293T cells were seeded at 7×10⁶ cells/plate in 10 cm dishes. The next day, the cells were transfected with 12.5 μg pCAGGS plasmids encoding coronavirus spike proteins (CoV-2-SA18 WT or variants, SARS-SA19, or MERS-SA16). The following day, transfected cells were infected with rVSVAG-Luc/VSV-G seed virus (Kerafast dot com) at MOI=1. After 24 hrs, supernatant was collected and centrifuged at 1320 g 10 min. Supernatant was aliquoted into single-use vials and stored at −80° C.

rVSVAG-Luc Pseudovirus Neutralization Assay

Pseudovirus neutralization assay and IC50 calculation were performed as previously described in (PMID: 35603164) with slight adaptation: Vero E6 cells (ATCC) were used at 22,000 cells/well for MERS in addition to 293T ACE2 cells used as described for SARS-CoV-2 WT and variants and SARS-CoV.

### Generation of Authentic SARS-CoV and SARS-CoV2

All work with authentic SARS-CoV and SARS-CoV-2 viruses were completed in BSL-3 laboratories at United States Army Medical Research Institute of Infectious Diseases (USAMRIID) in accordance with federal and institutional biosafety standards and regulations as described before (Pande et al. 2022 13:864775).

### Authentic SARS-CoV and SARS-CoV-2 IFA Neutralization Assay

Authentic SARS-CoV/Urbani, and SARS-CoV-2 neutralization assays were completed at USAMRIID as described before. Briefly, viruses at a multiplicity of infection of 0.2, was incubated for 1 h at 37° C. with serial dilutions of monoclonal antibodies. Vero-E6 monolayers were inoculated with the antibody-virus mixture at 37° C. for 1 hour. Following incubation, viral inoculum was removed and fresh cell culture media was added for an additional 23 hours at 37° C. Cells were washed with PBS, fixed in 10% formalin, permeabilized with 0.2% Triton-X for 10 minutes, and treated with blocking solution. Detection of infected cells was accomplished using an anti-SARS-CoV or anti-SARS-CoV-2 nucleocapsid protein (Sino Biological) detection antibodies, and a goat a-rabbit secondary antibody conjugated to AlexaFluor488. Infected cells were determined using the Operetta high content imaging instrument and data analysis was performed using the Harmony software (Perkin Elmer).

### Hydrogen-Deuterium Exchange Mass Spectrometry

To map the binding epitope for S3_29, 11F5 and 6A1, HDX-MS experiments were carried out using a spike protein S2 domain (SARS-CoV2-PreS-S2-His) produced in house. The protein solution was 1.9 mg/mL (MW ˜60 kDa, equivalent to ˜30 μM) in 50 mM HEPES, 300 mM NaCl, and pH 7.5 buffer. To form S2: VHH complex, equal volume of 30 μM S2 was incubated with 30 μM VHH at 4° C. for 30 min as stock solution. HDX-MS experiments were performed using an automated HDX system (Waters Corporation, MA, USA). Six microliters of S2 or S2: VHH stock solution were diluted 10-fold with labeling buffer at 20° C. to initiate the deuterium exchange reaction. The labeling time points were 0, 10 seconds, 1, 10, and 100 minutes. At each time point, an aliquot was removed and an equal volume of quench buffer (8 M urea, 1 M TCEP in 100 mM phosphate buffer, pH 2.5) was mixed at 4° C. and immediately analyzed. Online digestion was performed using an immobilized protease type XIII/pepsin column (w/w, 1:1), 2.1×30 mm (NBA2014002, NovaBioAssays, LLC, MA, USA). The eluent was directed into a SYNAPT® G2 HDMS mass spectrometer for analysis in MS (e) mode over the m/z range of 300-2000. Data acquired from undeuterated samples including three replicates of spike S2 and S2: VHH complex were used to identify peptides through ProteinLynx Global SERVER™ (PLGS) 3.0 software. Raw data from all time points were analyzed using DynamX™ 3.0 to generate relative deuterium uptake level in each peptide, which was used to generate deuterium uptake graphs and difference maps.

To map the binding epitope for 7A9, HDX-MS experiments were performed using a spike CoV-2 RBD domain produced in house. The protein solution was 2.6 mg/mL (MW ˜31 kDa, equivalent to ˜ 83.5 μM) in 10 mM sodium phosphate, 75 mM sodium chloride, 3% sucrose, and pH 7.4 buffer. CoV-2 RBD and 7A9 VHH complex was prepared by mixing 15 μL 2.6 mg/mL recombinant CoV-2 RBD with 11.7 μL 3.89 mg/mL 7A9 VHH and 43.3 μL PBS. The recombinant CoV-2 RBD alone was prepared by mixing 15 μL 2.6 mg/mL recombinant CoV-2 RBD with 55 μL PBS. 10 μL of the recombinant CoV-2 RBD alone or RBD: VHH complex was incubated with 90 μL of control buffer (50 mM phosphate, 100 mM sodium chloride at pH 7.4) or deuterium oxide labeling buffer (50 mM sodium phosphate, 100 mM sodium chloride at pD 7.0). The labeling time was 0 s, 15 s, 60 s, 600 s, or 3600 s at 8° C. Hydrogen/deuterium exchange was quenched by adding 100 μL of 4 M guanidine HCl, 0.85 M TCEP buffer (final pH was 2.5). Then, the mixture was subjected to on-column digestion using the protease type XIII/pepsin column. The resultant peptides were trapped and desalted on a Waters™ ACQUITY UPLC BEH C18 VanGuard pre-column (130 Å, 1.7 μm, 2.1 mm×5 mm, 186003975, Waters) for 3.5 min at 160 μL/min. Peptides were then eluted from the trap using a 2-30% gradient of acetonitrile (with 0.3% formic acid) over 12.5 min at a flow rate of 150 μL/min and are separated on a 50×1 mm C8 column (3 μm, NBA2014015, NovaBioAssays). A UPLC-MS system comprised of a Waters™ Acquity UPLC coupled to a Q Exactive™ HF Hybrid Quadrupole-Orbitrap Mass Spectrometer (Thermo) was used. Solvent A was 0.3% formic acid in water. The injection valve and enzyme column and their related connecting tubings were inside a cooling box maintained at 8° C. The second switching valve, C8 column and their related connecting stainless steel tubings were inside another chilled circulating box maintained at −6° C. Peptide identification was done through searching MS/MS data against the CoV-2 RBD sequence using Byonics (Protein Metrics, CA, USA). The mass tolerance for the precursor and product ions were 10 ppm and 0.02 Da, respectively. The mass spectra for deuterated samples were recorded in MS only mode. Raw MS data was processed using HDX WorkBench, software for the analysis of H/D exchange MS data (Pascal et al. 2012 23:1512-1521). The deuterium levels were calculated using the average mass difference between the deuterated peptide and its undeuterated form (to).

### Crystallization, Data Collection, and Structure Determination

The RBD-7A9 complex at 9.5 mg/mL was treated overnight with EndoH at 4° C. and used in crystallization trials without further purification. Crystals of the space group P3221 were grown at 20° C. by sitting drop vapor diffusion using a drop ratio of 2:1 protein: reservoir solution. Reservoir solution contained 0.2 M sodium malonate pH 6.0 and 18% PEG 3350. Crystals were cryoprotected in reservoir solution supplemented with 25% glycerol.

X-Ray diffraction data were collected at beamline 17-ID at the Advanced Photon Source. Data were processed using autoPROC (Vonrhein et al. 2011 67:293-302) and elliptically truncated using STARANISO (Tickle et al. 2018 Global Phasing Ltd.). The structure was solved by molecular replacement in Phenix (Adams et al. 2010 66:213-221) using the SARS-CoV-2 RBD (PDB 7E7Y; (Cao et al. 2021 31:732-741)) and a homology model of 7A9 with the CDRs removed as search models. There was one copy of the complex in the asymmetric unit. The structure was then rebuilt in Coot (Emsley and Cowtan 2004 60:2126-2132) and subjected to iterative rounds of refinement and rebuilding using Phenix and Coot. Data processing and refinement statistics are summarized in Table 8.

TABLE 8
Crystallographic data collection and refinement statistics

| | RBD-7A9 | |
|---|---|---|
| Data collection | | |
| Space group | P 3₂ 2 1 | |
| Cell dimensions | | |
| a, b, c (Å) | 93.90, 93.91, 121.39 | |
| a, b, g (°) | 90, 90, 120 | |
| Resolution (Å) | 81.32-2.35 (isotropic) | |
| | 81.32-2.01 (ellipsoidal) (2.22-2.01)* | |
| R_{sym} or R_{merge} | 0.069 | (0.959) |
| I/sI | 18.3 | (1.7) |
| Completeness (%) | 95.4 | (74.8) |
| Redundancy | 9.9 | (6.9) |
| Refinement | | |
| Resolution (Å) | 36.23-2.01 (anisotropic) | |
| No. reflections | 29572 | (1476) |

| | |
|---|---|
| R_{work}/R_{free} | 0.1691/0.2004 |
| No. atoms | |
| Protein | 2567 |
| Ligand/ion | 38 |
| Water | 158 |
| B-factors | |
| Protein | 51.20 |
| Ligand/ion | 102.79 |
| Water | 48.61 |
| R.m.s. deviations | |
| Bond lengths (Å) | 0.005 |
| Bond angles (°) | 0.771 |

*Values in parentheses are for highest-resolution shell

### Analytical Ultracentrifugation

For AUC experiments, we used the CoV-2-PreS-Closed Spike protein ectodomain, as described in the “Plasmid Construction, Protein Expression and Protein Purification” Methods section. This construct contains four stabilizing amino acid substitutions (D614N, A892P, A942P, and V987P), and forms a stable trimer without an artificial trimerization sequence. Samples contained 6 μM 7A9 or 1E4 VHH and/or 1.33 μM CoV-2-PreS-Closed Spike trimer (4 μM monomer) in AUC buffer (50 mM HEPES pH 7.5+150 mM NaCl). Samples were mixed and then incubated on a rotating platform at room temperature for 3 hours. 0.4 mL of each sample was loaded into the right side and 0.4 mL of AUC buffer was loaded into the left side of an AUC cell containing sapphire windows and a double sector charcoal EPON centerpiece. Balanced cells were loaded into an An50Ti rotor and thermally equilibrated in the chamber of a Beckman Optima AUC under vacuum at 25° C. for 1 hour. Samples were centrifuged at 40,000 RPM with a total of 300 scans taken at 40 second intervals using absorption optics set at 280 nm. Values for buffer density, viscosity, and partial specific volumes of the proteins were calculated in Sednterp software (Hayes, D.B., Laue, T., and Philo, J. 1999. Sedimentation Interpretation Program. University of New Hampshire.). Every 5th scan (60 total) was loaded into the program Sedfit for continuous distribution (c(s)) analysis (Brown and Schuck 2006 90:4651-4661). Initial fitting of parameters was iteratively performed to minimize r.m.s.d. values. After testing multiple values, the frictional coefficient was held constant at 1.2 for all samples. After optimizing fit parameters using 60 scans, all 300 scans were analyzed and peaks were integrated in Sedfit. All AUC figures were generated using Matplotlib software (Hunter 2007 9:90-95).

### Multimer Design

Following domain binning and HDX epitope mapping, the approximate epitope and binding location of many candidate monomeric VHHs against the spike protein were determined. To calculate the length of the desired linker between two VHHs, the distance between the center of the known epitope was measured in both the open and closed state of the spike protein in MOE 2020.09. To account for cases where the shortest distance between two sites would result in a clash and to account for the unknown binding orientation of the VHH in relation to the spike protein (where the terminus would be located), an additional buffer distance was added to the initial measurement. Given that an amino acid can cover 3.5-4.0 angstroms in an extended conformation, the measured distance was then converted to a minimum number of amino acids that would be required to bridge such a distance. The resulting number of amino acids was then further converted to the minimum number of linker subunits (e.g., G4S (GGGGS) (SEQ ID NO: 256)) that could connect the two sites.

### Exemplary Embodiments

Some of the contemplated embodiments include the following:
1. A polypeptide that binds to spike proteins from at least two different coronaviruses, the polypeptide comprising, in N to C order, the regions FR1, CDR1, FR2, CDR2, FR3, CDR3, and FR4, wherein
   (a) said CDR1 comprises the sequence of any one of SEQ ID NOs 31 to 54,
   said CDR2 comprises the sequence of any one of SEQ ID NOs 61 to 84, and
   said CDR3 comprises the sequence of any one of SEQ ID NOs 91 to 114; or
   (b) said CDR1, CDR2, and CDR3 respectively comprise the sequence of any one of SEQ ID NOs 31 to 54, 61 to 84, and 91 to 114 with one to three total residue mutations among themselves.
2. The polypeptide of embodiment 1, wherein said one to three residue mutations comprise at least one substitution, wherein said substitution is a conservative substitution.
3. The polypeptide of embodiment 1, wherein each of said one to three residue mutations is a conservative substitution.
4. The polypeptide of embodiment 1, wherein
   said CDR1 comprises the sequence of any one of SEQ ID NOs 31 to 54,
   said CDR2 comprises the sequence of any one of SEQ ID NOs 61 to 84, and
   said CDR3 comprises the sequence of any one of SEQ ID NOs 91 to 114.
5. The polypeptide of any one of embodiments 1 to 4, wherein the SEQ ID NOs of the sequences of said CDR1, CDR2, and CDR3 are congruent with each other in modulo 30.
6. The polypeptide of any one of embodiments 1 to 5, wherein
   (a) said FR1 comprises the sequence of any one of SEQ ID NOs 121 to 146,
   said FR2 comprises the sequence of any one of SEQ ID NOs 151 to 176,
   said FR3 comprises the sequence of any one of SEQ ID NOs 181 to 206, or
   said FR4 comprises the sequence of any one of SEQ ID NOs 211 to 236; or
   (b) said FR1, FR2, FR3, and FR4 respectively comprise the sequence of any one of SEQ ID NOs 121 to 144, 151 to 174, 181 to 204, and 211 to 234 with one to nine total residue mutations among themselves.
7. The polypeptide of any one of embodiments 1 to 5, wherein
   (a) said FR1 comprises the sequence of any one of SEQ ID NOs 121 to 146,
   said FR2 comprises the sequence of any one of SEQ ID NOs 151 to 176,
   said FR3 comprises the sequence of any one of SEQ ID NOs 181 to 206, and
   said FR4 comprises the sequence of any one of SEQ ID NOs 211 to 236; or
   (b) said FR1, FR2, FR3, and FR4 respectively comprise the sequence of any one of SEQ ID NOs 121 to 144, 151 to 174, 181 to 204, and 211 to 234 with one to nine total residue mutations among themselves.
8. The polypeptide of embodiment 6 or 7, wherein said one to nine residue mutations comprise at least one substitution, wherein said substitution is a conservative substitution.
9. The polypeptide of embodiment 6 or 7, wherein each of said one to nine residue mutations is a conservative substitution.
10. The polypeptide of any one of embodiments 1 to 5, wherein
   said FR1 comprises the sequence of any one of SEQ ID NOs 121 to 146,
   said FR2 comprises the sequence of any one of SEQ ID NOs 151 to 176,
   said FR3 comprises the sequence of any one of SEQ ID NOs 181 to 206, and
   said FR4 comprises the sequence of any one of SEQ ID NOs 211 to 236.
11. The polypeptide of any one of embodiments 6 to 10, wherein the SEQ ID NOs of the sequences of said FR1, FR2, FR3, and FR4 are congruent with each other in modulo 30.
12. The polypeptide of any one of embodiments 1 to 11, wherein said regions are in said N to C order contiguously.
13. The polypeptide of any one of embodiments 1 to 5, comprising a sequence that has at least 90, 91, 92, 93, 94, 95, 96, 97, 98, or 99% identity with the entire sequence of any one of SEQ ID NOs 1 to 24.
14. A polypeptide that binds to spike proteins from at least two different coronaviruses, comprising the sequence of any one of SEQ ID NOs 1 to 24.
15. The polypeptide of any one of embodiments 1 to 14, wherein said different coronaviruses comprise different species selected from SARS-CoV, SARS-CoV2, and MERS-CoV.
16. The polypeptide of embodiment 15, wherein said different coronaviruses consist of SARS-CoV and SARS-CoV2.
17. The polypeptide of any one of embodiments 1 to 16, wherein said different coronaviruses comprise different SARS-CoV2 variants selected from B.1.1.7, B.1.351, P.1, B.1.617.2, and B.1.1.529.
18. The polypeptide of any one of embodiments 1 to 17, wherein the polypeptide binds to each of said spike proteins, with an EC50 value that is numerically lower than 100 nM as measured by an ELISA assay.
19. The polypeptide of any one of embodiments 1 to 17, wherein the polypeptide binds to each of said spike proteins with a K_{D} value that is numerically lower than 100 nM as measured by a surface plasmon resonance (SPR) assay.
20. The polypeptide of embodiment 18 or 19, wherein the polypeptide is in a monovalent form.
21. The polypeptide of any one of embodiments 1 to 20, wherein the polypeptide is a single-domain antibody, a single-chain variable fragment, an antibody, a Fab fragment, a F(ab′)2 fragment, a Fab′ fragment, or an Fv fragment.
22. The polypeptide of any one of embodiments 1 to 21, wherein said polypeptide inhibits infection of Vero-E6 cells by said at least two different coronaviruses with an IC50 value that is numerically lower than 100 nM.
23. A single-domain antibody comprising a CDR1 having the sequence of any one of SEQ ID NOs 31 to 54; a CDR2 having the sequence of any one of SEQ ID NOs 61 to 84; and a CDR3 having the sequence of any one of SEQ ID NOs 91 to 114, wherein the SEQ ID NOs of the sequences of said CDR1, CDR2, and CDR3 are congruent with each other in modulo 30.
24. The single-domain antibody of embodiment 23, comprising a sequence that has at least 90, 91, 92, 93, 94, 95, 96, 97, 98, or 99% identity with the entire sequence of any one of SEQ ID NOs 1 to 24.
25. The single-domain antibody of embodiment 23, comprising the sequence of any one of SEQ ID NOs 1 to 24.
26. The single-domain antibody of any one of embodiments 23 to 25, wherein the single-domain antibody, when in a monovalent form, binds to at least two spike proteins with a K_{D} value that is numerically lower than 100 nM as measured by SPR, wherein said at least two spike proteins are from different coronaviruses selected from SARS-CoV, SARS-CoV2, and MERS-CoV.
27. The single-domain antibody of any one of embodiments 23 to 26, wherein the single domain antibody when in a monovalent form, binds to at least two spike proteins with a K_{D} value that is numerically lower than 100 nM as measured by SPR, wherein said at least two spike proteins are from at least two different coronaviruses selected from SARS-CoV2 variants B.1.1.7, B.1.351, P.1, B.1.617.2, and B.1.1.529.
28. The single-domain antibody of any one of embodiments 23 to 25, wherein said single-domain antibody, when in a monovalent form, inhibits infection of Vero-E6 cells by at least two different coronaviruses with an IC50 value that is numerically lower than 100 nM, wherein said coronaviruses are selected from SARS-CoV, SARS-CoV2, and MERS-CoV.
29. The single-domain antibody of any one of embodiments 23 to 25 and 28, wherein said single domain antibody when in a monovalent form separately inhibits infection of Vero-E6 cells by at least two different coronaviruses, with an IC50 value that is numerically lower than 100 nM, wherein said coronaviruses are selected from SARS-CoV2 variants B.1.1.7, B.1.351, P.1, B.1.617.2, and B.1.1.529.
30. A single-domain antibody that binds to the same epitope as the polypeptide of any one of embodiments 1 to 22 or as the single-domain antibody of any one of embodiments 23 to 29.
31. The single-domain antibody of embodiment 30, wherein said epitope is on the N-terminal domain (NTD), S2 domain, or receptor binding domain (RBD) of a coronavirus spike protein.
32. The single-domain antibody of embodiment 30, wherein said epitope is on the apical end of the NTD of a coronavirus spike protein.
33. The single-domain antibody of embodiment 30, wherein said epitope comprises the sequence of any one of SEQ ID NOs 241 to 249 or comprises the sequence of residues 1176-1178 of SEQ ID NO: 240.
34. The single-domain antibody of any one of embodiments 30 to 33, wherein said epitope is determined via hydrogen-deuterium exchange mass spectrometry.
35. A polypeptide comprising two spike-protein binders each independently selected from the polypeptides of any one of embodiments 1 to 22 and the single-domain antibodies of any one of embodiments 23 to 34.
36. The polypeptide of embodiment 35, further comprising a linker between the two spike-protein binders.
37. The polypeptide of embodiment 36, wherein the linker comprises 10 to 90 amino acids.
38. A polypeptide comprising a first spike-protein binder, a second spike-protein binder, and a third spike-protein binder, wherein each spike-protein binder is independently selected from the polypeptides of any one of embodiments 1 to 22 and the single-domain antibodies of any one of embodiments 23 to 34.
39. The polypeptide of embodiment 38, further comprising a first linker between the first spike-protein binder and the second spike-protein binder.
40. The polypeptide of embodiment 38 or 39, further comprising a second linker between the second spike-protein binder and the third spike-protein binder.
41. The polypeptide of embodiment 39 or 40, wherein the first linker comprises 10 to 30 amino acids.
42. The polypeptide of any one of embodiments 38 to 41, wherein the second linker comprises 10 to 70 amino acids.
43. The polypeptide of any one of embodiments 38 to 42, wherein the spike-protein binders independently bind to an NTD, an S2, or an RBD of the spike protein.
44. The polypeptide of embodiment 43, wherein each spike-protein binder binds to an RBD of the spike protein.
45. The polypeptide of embodiment 44, wherein each spike-protein binder binds to the RBD of a different monomer of the spike protein.
46. The polypeptide of any one of embodiments 38 to 43, wherein the spike-protein binders collectively bind to an NTD, an S2, and an RBD of the spike protein.
47. The polypeptide of embodiment 46, wherein the first spike-protein binder binds to an RTD, the second spike-protein binder binds to an NTD, and the third spike-protein binder binds to the S2 of the spike protein.
48. The polypeptide of any one of embodiments 38 to 42, wherein the first spike-protein binder, the second spike-protein binder, and the third spike-protein binder respectively comprise CDR3s having the sequence of the following SEQ ID NOs:
   107-91-111;
   91-111-109;
   107-92-114;
   92-107-114;
   91-107-105;
   91-111-105;
   91-107-111;
   91-111-114;
   107-91-105;
   107-111-105;
   107-111-109;
   107-111-114;
   111-105-107;
   111-105-105;
   105-105-107;
   105-111-114;
   105-105-114;
   105-99-114;
   113-105-114;
   113-105-107;
   113-111-114;
   111-105-113;
   113-99-114; or
   113-105-113,
wherein the first spike-protein binder, the second spike-protein binder, and the third spike-protein binder further comprise CDR1s having the sequence of any one of SEQ ID NOs 31 to 54, and CDR2s having the sequence of any one of SEQ ID NOs 61 to 84, wherein the SEQ ID NOs of the sequences of said CDR1, CDR2, and CDR3 are congruent with each other in modulo 30.
49. The polypeptide of any one of embodiments 38 to 42, wherein the first spike-protein binder, the second spike-protein binder, and the third spike-protein binder respectively comprise the sequences of the following SEQ ID NOS:
   17-1-21;
   1-21-2019;
   17-2-24;
   2-17-2024;
   1-17-2015;
   1-21-2015;
   1-17-2021;
   1-21-2024;
   17-1-15;
   17-21-15;
   17-21-19;
   17-21-24;
   21-15-17;
   21-15-15;
   15-15-17;
   15-21-24;
   15-15-24;
   15-9-24;
   23-15-24;
   23-15-17;
   23-21-24;
   21-15-23;
   23-9-24; or
   23-15-23.
50. A polypeptide comprising four or more spike-protein binders each independently selected from the polypeptides of any one of embodiments 1 to 22 and the single-domain antibodies of any one of embodiments 23 to 34.
51. The polypeptide of any one of embodiments 35 to 50, wherein said polypeptide separately exhibits neutralization potencies against infection of Vero-E6 cells by at least two different coronaviruses selected from SARS-CoV, SARS-CoV2, and MERS-CoV, wherein said neutralization potencies measured as IC50 values are numerically lower than those for a mixture of corresponding spike-protein binders.
52. The polypeptide of any one of embodiments 35 to 50, wherein said polypeptide separately exhibits neutralization potencies against infection of Vero-E6 cells by at least two different coronaviruses selected from SARS-CoV2 variants B.1.1.7, B.1.351, P.1, B.1.617.2, and B.1.1.529, wherein said neutralization potencies measured as IC50 values are numerically lower than those for a mixture of corresponding spike-protein binders.
53. A polypeptide consisting of an antigen-binding fragment of the polypeptide of any one of embodiments 1 to 22 and 35 to 52 or of the single-domain antibody of any one of embodiments 23 to 34.
54. A polypeptide that competes with the polypeptide of any one of embodiments 1 to 22 and 35 to 53 or the single-domain antibody of any one of embodiments 23 to 34 for binding to a coronavirus spike protein.
55. A composition comprising the polypeptide of any one of embodiments 1 to 22 and 35 to 54 or the single-domain antibody of any one of embodiments 23 to 34 and a pharmaceutically acceptable carrier.
56. A kit comprising the composition of embodiment 55.
57. The kit of embodiment 56, wherein the composition is contained within an injection device or glass vial.
58. The kit of embodiment 56 or 57 further comprising a second therapeutic agent or vaccine.
59. An isolated nucleic acid encoding the polypeptide of any one of embodiments 1 to 22 and 35 to 54 or the single-domain antibody of any one of embodiments 23 to 34.
60. The isolated nucleic acid of embodiment 59, wherein said nucleic acid is a DNA.
61. An expression vector comprising the nucleic acid of embodiment 59 or 60.
62. A host cell comprising the expression vector of embodiment 61.
63. A conjugate comprising the polypeptide of any one of embodiments 1 to 22 and 35 to 54 or the single-domain antibody of any one of embodiments 23 to 34, and a therapeutic agent.
64. The conjugate of embodiment 63, wherein the therapeutic agent comprises an antibody or fragment thereof, an immunomodulator, a hormone, a cytotoxic agent, an enzyme, a radionuclide, an antibody conjugated to at least one immunomodulator, enzyme, radioactive label, hormone, antisense oligonucleotide, or cytotoxic agent, or a combination thereof.
65. A conjugate comprising the polypeptide of any one of embodiments 1 to 22 and 35 to 54 or the single-domain antibody of any one of embodiments 23 to 34, and a half-life extender.
66. The conjugate of embodiment 65, wherein the half-life extender comprises a heavy chain constant domain or a crystallizable fragment domain.
67. A method for producing the polypeptide of any one of embodiments 1 to 22 and 35 to 54 or the single-domain antibody of any one of embodiments 23 to 34, comprising cultivating the host cell of embodiment 62 in a medium under conditions suitable for expression of the polypeptide or single-domain antibody by the host cell; and isolating the polypeptide or single-domain antibody from the medium.
68. A method of neutralizing a coronavirus in a sample, comprising contacting the sample with an effective amount of the polypeptide of any one of embodiments 1 to 22 and 35 to 54 or the single-domain antibody of any one of embodiments 23 to 34.
69. The method of embodiment 68, wherein the coronavirus is SARS-CoV, SARS-CoV2, or MERS-CoV.
70. The method of embodiment 68 or 69, wherein the coronavirus is a SARS-CoV2 variant selected from B.1.1.7, B.1.351, P.1, B. 1.617.2, and B.1.1.529.
71. A method of treating a coronavirus infection in a subject, comprising administering to a subject in need thereof an effective amount of the polypeptide of any one of embodiments 1 to 22 and 35 to 54 or the single-domain antibody of any one of embodiments 23 to 34.
72. The method of embodiment 71, wherein the coronavirus infection is of SARS-CoV, SARS-CoV2, or MERS-CoV.
73. A method of treating a coronavirus disease in a subject, comprising administering to a subject in need thereof an effective amount of a composition comprising the polypeptide of any one of embodiments 1 to 22 and 35 to 54 or the single-domain antibody of any one of embodiments 23 to 34.
74. The method of embodiment 73, wherein the coronavirus disease is SARS, MERS, or COVID-19.
75. A polypeptide of any one of embodiments 1 to 22 and 35 to 54 or the single-domain antibody of any one of embodiments 23 to 34 for use in the treatment of a coronavirus infection.
76. The polypeptide or single-domain antibody of embodiment 75, wherein the coronavirus infection is of SARS-CoV, SARS-CoV2, or MERS-CoV.
77. A composition comprising the polypeptide of any one of embodiments 1 to 22 and 35 to 54 or the single-domain antibody of any one of embodiments 23 to 34 for use in the treatment of a coronavirus disease.
78. The composition of embodiment 77, wherein the coronavirus disease is SARS, MERS, or COVID-19.
79. Use of a polypeptide of any one of embodiments 1 to 22 and 35 to 54 or the single-domain antibody of any one of embodiments 23 to 34 for the treatment of a coronavirus infection.
80. The use of embodiment 79, wherein the coronavirus infection is of SARS-CoV, SARS-CoV2, or MERS-CoV.
81. Use of a composition comprising the polypeptide of any one of embodiments 1 to 22 and 35 to 54 or the single-domain antibody of any one of embodiments 23 to 34 for the treatment of a coronavirus disease.
82. The use of embodiment 81, wherein the coronavirus disease is SARS, MERS, or COVID-19.
83. Use of a polypeptide of any one of embodiments 1 to 22 and 34 to 54 or the single-domain antibody of any one of embodiments 23 to 34 in the manufacture of a medicament for the treatment of a coronavirus infection.
84. The use of the polypeptide or single-domain antibody of embodiment 83, wherein the coronavirus infection is of SARS-CoV, SARS-CoV2, or MERS-CoV.
85. Use of a composition comprising the polypeptide of any one of embodiments 1 to 22 and 35 to 54 or the single-domain antibody of any one of embodiments 23 to 34 in the manufacture of a medicament for the treatment of a coronavirus disease.
86. The use of embodiment 85, wherein the coronavirus disease is SARS, MERS, or COVID-19.

### SEQUENCE LISTING

A sequence listing file with SEQ ID NOs 1 to 256 is separately filed on the same date as the filing of this application, which is part of the specification as stated in the section titled “reference to sequence listing submitted electronically.” The alternatively formatted listing below only provides the sequences of the SEQ ID NOs 1 to 24.

>SEQ ID NO: 1
QVQLVESGGGLVQPGGSLRLSCAASGITLDYYAIAWFRQAPGKEREGVSCFCSSDGDIR
YADSVKGRFTISRDNAKNTVYLQMNSLKPEDTAVYYCATERVYSSTWFFHRYSELDFG
SWGRGTQVTVSS
>SEQ ID NO: 2
QVLLVESGGGLVQAGGLLRLACSASGRTFTENVMGWFRQAPGKEREFVAAINWNTGST
YYSDSRKGRFTISKDNAKNTMFLQINNLKPEDTAVYYCGGTSSSYYYIGEFIPDYWGQG
TQVTVSS
>SEQ ID NO: 3
QVQLVESGGGLVQAGGSLRLSCAASGRAFSKDTMGWFRQAPGKEREFVAVISASGDST
YYADFVKGRFTISRDNAKNTEYLQMNSLKPEDTAVYHCAADGARAYYSINAHRYSWA
YNYWGQGTQVTVSS
>SEQ ID NO: 4
QVQLVESGGGLVQAGGSLRLSCAASGRTFSIYAVGWFRQAPGKERDFVAGITWSGGTT
YYSDSVKGRFTISRDNAKNTVYLQMNNLKPEDTAIYYCAVDTYSMTLSARSYDYWGQG
TQVTVSS
>SEQ ID NO: 5
QVQLVESGGGLVQAGGSLRLSCAASGRTVNKYMTGWFRQTPGKEREFVASIDWSSTTT
HYANSVKGRFTISRDKVKNTVYLQMNGLKPEDTAVYYCAAHQDSSYGYTILPIDYDSW
GQGTQVTVSS
>SEQ ID NO: 6
QVQLVESGGGLVQPGGSLRLSCAASGFTLDLNSIGWFRQAPGKEREGVSCISPSGSRMH
YADSVKGRFTISRDNAKNTVYLQMNSLKPEDTAVYYCATGSPSYHYCSMYGLEYDSW
GQGTQVTVPS
>SEQ ID NO: 7
QVQLVESGGGLAQAGGSLRLSCASSGLIFSGSAMGWFRQAPGKEREFVGVISWNGRTTY
ADSVKGRFTISRDDAVNMVWLQMNNLKPEDTAVYYCAADHLNYGSGDVNPRTYDYW
GQGTQVTVSS
>SEQ ID NO: 8
QVQLVESGGGLVQPGGSLRLSCARSENNFSINAMAWYRQAPGKQRELVAAITTAGTTN
YADSVKGRFTISRDNAKTTIYLQMNSLKPEDTAVYYCNLWAGTRDDLNEYWGQGTQV
TVSS
>SEQ ID NO: 9
QVQLVESGGGAVQAGDSLRLSCTASGRTFSTYAVGWFRQAPGKERELLAAITGSGRNP
YYPDSLKGRFTISREYAKLPVYLQMNSLKPEDTAVYYCAARRGAIATMPLDYDHWGQG
TQVTVSS
>SEQ ID NO: 10
QVQLVESGGGLVQAGGSLRLSCEVSGSIYSINSMGWYRQAPGKQRELVAGIVSDGRINY
ADSVKGRFTISRAKNTVYLQMNSLKPEDTAVYYCAADRAFVYAGDYDYLGQGTQVTV
SS
>SEQ ID NO: 11
QVQLVESGGGLVQAGGSLRLSCAASGRTSSIYGVGWFRQAPGKERELVALITWSGGTTY
YSDSVKGRFTISKDNAKNTVFLQMNNLKPEDTAIYYCAVDTYSMTMSARDYEYWGQG
TQVTVSS
>SEQ ID NO: 12
QVQLVESGGRLVQAGGSLRLSCAAPGRTFRNLTLGWFRQAPGKEREFVASTSWPDDEM
TYYTDSVKGRFTISRDNAKNTVYLQMNSLKPEDTAVYYCAAASWSGRSTLSYDYDYW
GQGTQVTVST
>SEQ ID NO: 13
QVQLVESGGGLVQAGGSLRLSCAASGRTVGYYAVGWFRQAPGKERDFVAAISWSGRN
TNYADSVTGRFTIFRDNAKNTVDLQMNSLKPEDTAVYYCAADYWNYGSGSEVPNNYD
TWGQGTQVTVSS
>SEQ ID NO: 14
QVQLVESGGGLVQPGGSLRLSCAASGFTLDLNSIGWFRQAPGKEREGVSCISPSASSTHY
ADSVKGRFTISRDNAKNTVYLQMNSLKPEDTAVYYCATGTPSYHYCSMYGLEYDDWG
QGTQVTVSS
>SEQ ID NO: 15
QVQLVESGGGLVQPGGSLRLSCARSENNFSINAMAWYRQAPGKQRELVAAITTSGTTNY
ADSVKGRFTISRDNAKTTIYLQMNSLKPEDTAVYYCNLWAGTRDDLNEYWGQGTQVT
VSS
>SEQ ID NO: 16
QVQLVESGGGLVEAGGSLRLSCVASGSSSSINAMGWYRQAPGKQRELVASISHDGETKY
ANSVKGRFTISRGNAKNTIYLQMNSLKPEDTAVYYCVGDVAWVVVAGEYDYWGQGTQ
VTVSS
>SEQ ID NO: 17
QVQLVESGGGLVQAGGSLRLSCSASGGTASRSAMGWFRQAPGKEREFVAGISRRNSGST
YVADSYEDSVKGRFTISRDNAKNTIYLQMNSLKPEDTAVYYCAAEPTLGWYVPRRSVE
YEYWGQGTQVTVSS
>SEQ ID NO: 18
QVQLVESGGGLVQAGGSLRLSCAASGRTGSRYIMGWFRQAPGKEREFVAAINWRGDST
YYADSVKGRFTIARDNAKNTAYLQMNSLKPEDTAVYYCVADRGESYYYTRSTEYTYW
GQGTQVTVSS
>SEQ ID NO: 19
QVQLVESGGGLVQAGGSLRLSCAASGRTSSIYSVGWFRQAPGKERELVAGITWSGGTTY
YSDSVKGRFTISRDNAKNTVNLQMNNLKPEDTAIYYCAVDRYSMTMSARDYDYWGQG
TQVTVSS
>SEQ ID NO: 20
QVQLVESGGGLVQPGGSLRLSCAASGFTLDNYAIGWFRQAPGKEREGVSCISSSDGDTQ
YADSVKGRFTISRDNAKNTVYLQMNSLKPEDTAVYYCAVEKVYARSWFFHLCSELDFG
SWGQGTQVTVSS
>SEQ ID NO: 21
QVQLVESGGGLVQAGGSLRLSCAASGRTFTNYAMGWFRQAPGKEREFVAAISWSGGGT
NYADSVKGRFTISRDNAKNTVYLQMNSPKPEDTAVYYCATTLGYYGSSSRLYEYWGQG
TQVTVSS
>SEQ ID NO: 22
QVQLVESGGGLAQAGGSLRLSCATSGLTFSMSAMGWFRQAPGNEREFVGVINWNGGSS
RYADSVRGRFTISRDNAKNTVYLQMNSLKPEDTGVYYCAADALNIGTGEVNPRLYDYW
GQGTQVMVSS
>SEQ ID NO: 23
QVQLVESGGGLVQPGGSLRLSCVASGSTISINSMGWYRQAPGKAREMVATITNDGPIKY
ADPVKGRFTISRDNPKNTVYLQMNSLKPEDTAVYYCVADRAYWLAGEWEYWGQGTQ
VTVSS
>SEQ ID NO: 24
QVQLVESGGGLVQAGGSLRLSCTASGNIFSVNVMGWHRQAPGKRRELVAALTNDGRR
NYADSVKGRFTISRDNAKNTIYLEMNSLKPEDTAVYYCGTGTLESGENSWGQGLQVTVS
S

All references cited herein are incorporated by reference to the same extent as if each individual publication, database entry (e.g., GenBank sequences or GeneID entries), patent application, or patent, was specifically and individually indicated to be incorporated by reference. This statement of incorporation by reference is intended by Applicant, pursuant to 37 C.F.R. § 1.57 (b) (1), to relate to each and every individual publication, database entry (e.g., GenBank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A single-domain antibody comprising a CDR1 having the sequence of any one of SEQ ID NOs 31 to 54; a CDR2 having the sequence of any one of SEQ ID NOs 61 to 84; and a CDR3 having the sequence of any one of SEQ ID NOs 91 to 114, wherein the SEQ ID NOs of the sequences of said CDR1, CDR2, and CDR3 are congruent with each other in modulo 30, and wherein the single-domain antibody, when in a bivalent form, can separately bind to at least two spike proteins with a K_{D} value that is numerically lower than 1 nanomolar as measured by surface plasmon resonance.

2. The single-domain antibody of claim 1, comprising a sequence that has at least 90% identity with the entire sequence of any one of SEQ ID NOs 1 to 24 or the sequence of any one of SEQ ID NOs 1 to 2.

3. (canceled)

4. The single-domain antibody of claim 1, wherein said at least two spike proteins are from different coronaviruses selected from SARS-CoV, SARS-CoV2, and MERS-CoV.

5. The single-domain antibody of claim 1, wherein said at least two spike proteins are from at least two different coronaviruses selected from SARS-CoV2 variants B.1.1.7, B.1.351, P.1, B.1.617.2, and B.1.1.529.

6. The single-domain antibody of claim 1, wherein the single-domain antibody, when in a bivalent form, can separately inhibit infection of Vero-E6 cells by at least two different coronaviruses with an IC50 value that is numerically lower than 10 nanomolar, wherein said coronaviruses are selected from SARS-CoV, SARS-CoV2, and MERS-CoV or said coronaviruses are selected from SARS-CoV2 variants B.1.1.7, B.1.351, P.1, B.1.617.2, and B.1.1.529.

7. (canceled)

8. A polypeptide comprising a first spike-protein binder, a second spike-protein binder, and a third spike-protein binder, wherein each spike-protein binder is independently selected from the single-domain antibodies of claim 1.

9. The polypeptide of claim 8, further comprising a first linker between the first spike-protein binder and the second spike-protein binder, optionally wherein the first linker comprises 10 to 30 amino acids.

10. (canceled)

11. The polypeptide of claim 9, further comprising a second linker between the second spike-protein binder and the third spike-protein binder, optionally wherein the second linker comprises 10 to 70 amino acids.

12. (canceled)

13. The polypeptide of claim 11, wherein the first spike-protein binder, the second spike-protein binder, and the third spike-protein binder respectively comprise CDR3s having the sequence of the following SEQ ID NOs:
107-91-111;
91-111-109;
107-92-114;
92-107-114;
91-107-105;
91-111-105;
91-107-111;
91-111-114;
107-91-105;
107-111-105;
107-111-109;
107-111-114;
111-105-107;
111-105-105;
105-105-107;
105-111-114;
105-105-114;
105-99-114;
113-105-114;
113-105-107;
113-111-114;
111-105-113;
113-99-114; or
113-105-113,
wherein the first spike-protein binder, the second spike-protein binder, and the third spike-protein binder further comprise CDR1s having the sequence of any one of SEQ ID NOs 31 to 54, and CDR2s having the sequence of any one of SEQ ID NOs 61 to 84, wherein the SEQ ID NOS of the sequences of said CDR1, CDR2, and CDR3 are congruent with each other in modulo 30.

14. The polypeptide of claim 13, wherein the first spike-protein binder, the second spike-protein binder, and the third spike-protein binder respectively comprise the sequences of the following SEQ ID NOs:
17-1-21;
1-21-2019;
17-2-24;
2-17-2024;
1-17-2015;
1-21-2015;
1-17-2021;
1-21-2024;
17-1-15;
17-21-15;
17-21-19;
17-21-24;
21-15-17;
21-15-15;
15-15-17;
15-21-24;
15-15-24;
15-9-24;
23-15-24;
23-15-17;
23-21-24;
21-15-23;
23-9-24; or
23-15-23.

15. The polypeptide of claim 11, wherein the spike-protein binders independently bind to an N-terminal domain (NTD), an S2 domain, or a receptor binding domain (RBD) of the spike protein.

16. A polypeptide that binds to spike proteins from at least two different coronaviruses, comprising, in N to C order, the regions FR1, CDR1, FR2, CDR2, FR3, CDR3, and FR4, wherein
said CDR1 comprises the sequence of any one of SEQ ID NOs 31 to 54,
said CDR2 comprises the sequence of any one of SEQ ID NOs 61 to 84, and
said CDR3 comprises the sequence of any one of SEQ ID NOs 91 to 114,
wherein the SEQ ID NOs of the sequences of said CDR1, CDR2, and CDR3 are congruent with each other in modulo 30.

17. The polypeptide of claim 16, comprising a sequence that has at least 95% identity with the entire sequence of any one of SEQ ID NOs 1 to 24 or the sequence of any one of SEQ ID NOs 1 to 24.

18-20. (canceled)

21. A composition comprising the single-domain antibody of claim 1 and a pharmaceutically acceptable carrier.

22. A kit comprising the composition of claim 21.

23. An isolated nucleic acid encoding the single-domain antibody of claim 1.

24. An expression vector comprising the nucleic acid of claim 23.

25. A host cell comprising the expression vector of claim 24.

26. A method of producing the single-domain antibody comprising
cultivating the host cell of claim 25 in a medium under conditions suitable for expression of the polypeptide or single-domain antibody by the host cell; and
isolating the polypeptide or single-domain antibody from the medium.

27. A method of neutralizing or treating a coronavirus in a sample, comprising contacting the sample with an effective amount of the single-domain antibody of claim 1 or a composition comprising the single-domain antibody of claim 1.

28-29. (canceled)


SEQUENCE LISTING (256 sequences)

SEQ ID NO: 1 - protein, 129 aa, synthetic construct
QVQLVESGGGLVQPGGSLRLSCAASGITLDYYAIAWFRQAPGKEREGVSCFCSSDGDIRYADSVKGRFTISRDNAKNTVYLQMNSLKPEDTAVYYCATERVYSSTWFFHRYSELDFGSWGRGTQVTVSS

SEQ ID NO: 2 - protein, 124 aa, synthetic construct
QVLLVESGGGLVQAGGLLRLACSASGRTFTENVMGWFRQAPGKEREFVAAINWNTGSTYYSDSRKGRFTISKDNAKNTMFLQINNLKPEDTAVYYCGGTSSSYYYIGEFIPDYWGQGTQVTVSS

SEQ ID NO: 3 - protein, 129 aa, synthetic construct
QVQLVESGGGLVQAGGSLRLSCAASGRAFSKDTMGWFRQAPGKEREFVAVISASGDSTYYADFVKGRFTISRDNAKNTEYLQMNSLKPEDTAVYHCAADGARAYYSINAHRYSWAYNYWGQGTQVTVSS

SEQ ID NO: 4 - protein, 123 aa, synthetic construct
QVQLVESGGGLVQAGGSLRLSCAASGRTFSIYAVGWFRQAPGKERDFVAGITWSGGTTYYSDSVKGRFTISRDNAKNTVYLQMNNLKPEDTAIYYCAVDTYSMTLSARSYDYWGQGTQVTVSS

SEQ ID NO: 5 - protein, 126 aa, synthetic construct
QVQLVESGGGLVQAGGSLRLSCAASGRTVNKYMTGWFRQTPGKEREFVASIDWSSTTTHYANSVKGRFTISRDKVKNTVYLQMNGLKPEDTAVYYCAAHQDSSYGYTILPIDYDSWGQGTQVTVSS

SEQ ID NO: 6 - protein, 126 aa, synthetic construct
QVQLVESGGGLVQPGGSLRLSCAASGFTLDLNSIGWFRQAPGKEREGVSCISPSGSRMHYADSVKGRFTISRDNAKNTVYLQMNSLKPEDTAVYYCATGSPSYHYCSMYGLEYDSWGQGTQVTVPS

SEQ ID NO: 7 - protein, 125 aa, synthetic construct
QVQLVESGGGLAQAGGSLRLSCASSGLIFSGSAMGWFRQAPGKEREFVGVISWNGRTTYADSVKGRFTISRDDAVNMVWLQMNNLKPEDTAVYYCAADHLNYGSGDVNPRTYDYWGQGTQVTVSS

SEQ ID NO: 8 - protein, 119 aa, synthetic construct
QVQLVESGGGLVQPGGSLRLSCARSENNFSINAMAWYRQAPGKQRELVAAITTAGTTNYADSVKGRFTISRDNAKTTIYLQMNSLKPEDTAVYYCNLWAGTRDDLNEYWGQGTQVTVSS

SEQ ID NO: 9 - protein, 123 aa, synthetic construct
QVQLVESGGGAVQAGDSLRLSCTASGRTFSTYAVGWFRQAPGKERELLAAITGSGRNPYYPDSLKGRFTISREYAKLPVYLQMNSLKPEDTAVYYCAARRGAIATMPLDYDHWGQGTQVTVSS

SEQ ID NO: 10 - protein, 118 aa, synthetic construct
QVQLVESGGGLVQAGGSLRLSCEVSGSIYSINSMGWYRQAPGKQRELVAGIVSDGRINYADSVKGRFTISRAKNTVYLQMNSLKPEDTAVYYCAADRAFVYAGDYDYLGQGTQVTVSS

SEQ ID NO: 11 - protein, 123 aa, synthetic construct
QVQLVESGGGLVQAGGSLRLSCAASGRTSSIYGVGWFRQAPGKERELVALITWSGGTTYYSDSVKGRFTISKDNAKNTVFLQMNNLKPEDTAIYYCAVDTYSMTMSARDYEYWGQGTQVTVSS

SEQ ID NO: 12 - protein, 125 aa, synthetic construct
QVQLVESGGRLVQAGGSLRLSCAAPGRTFRNLTLGWFRQAPGKEREFVASTSWPDDEMTYYTDSVKGRFTISRDNAKNTVYLQMNSLKPEDTAVYYCAAASWSGRSTLSYDYDYWGQGTQVTVST

SEQ ID NO: 13 - protein, 126 aa, synthetic construct
QVQLVESGGGLVQAGGSLRLSCAASGRTVGYYAVGWFRQAPGKERDFVAAISWSGRNTNYADSVTGRFTIFRDNAKNTVDLQMNSLKPEDTAVYYCAADYWNYGSGSEVPNNYDTWGQGTQVTVSS

SEQ ID NO: 14 - protein, 126 aa, synthetic construct
QVQLVESGGGLVQPGGSLRLSCAASGFTLDLNSIGWFRQAPGKEREGVSCISPSASSTHYADSVKGRFTISRDNAKNTVYLQMNSLKPEDTAVYYCATGTPSYHYCSMYGLEYDDWGQGTQVTVSS

SEQ ID NO: 15 - protein, 119 aa, synthetic construct
QVQLVESGGGLVQPGGSLRLSCARSENNFSINAMAWYRQAPGKQRELVAAITTSGTTNYADSVKGRFTISRDNAKTTIYLQMNSLKPEDTAVYYCNLWAGTRDDLNEYWGQGTQVTVSS

SEQ ID NO: 16 - protein, 121 aa, synthetic construct
QVQLVESGGGLVEAGGSLRLSCVASGSSSSINAMGWYRQAPGKQRELVASISHDGETKYANSVKGRFTISRGNAKNTIYLQMNSLKPEDTAVYYCVGDVAWVVVAGEYDYWGQGTQVTVSS

SEQ ID NO: 17 - protein, 131 aa, synthetic construct
QVQLVESGGGLVQAGGSLRLSCSASGGTASRSAMGWFRQAPGKEREFVAGISRRNSGSTYVADSYEDSVKGRFTISRDNAKNTIYLQMNSLKPEDTAVYYCAAEPTLGWYVPRRSVEYEYWGQGTQVTVSS

SEQ ID NO: 18 - protein, 125 aa, synthetic construct
QVQLVESGGGLVQAGGSLRLSCAASGRTGSRYIMGWFRQAPGKEREFVAAINWRGDSTYYADSVKGRFTIARDNAKNTAYLQMNSLKPEDTAVYYCVADRGESYYYTRSTEYTYWGQGTQVTVSS

SEQ ID NO: 19 - protein, 123 aa, synthetic construct
QVQLVESGGGLVQAGGSLRLSCAASGRTSSIYSVGWFRQAPGKERELVAGITWSGGTTYYSDSVKGRFTISRDNAKNTVNLQMNNLKPEDTAIYYCAVDRYSMTMSARDYDYWGQGTQVTVSS

SEQ ID NO: 20 - protein, 129 aa, synthetic construct
QVQLVESGGGLVQPGGSLRLSCAASGFTLDNYAIGWFRQAPGKEREGVSCISSSDGDTQYADSVKGRFTISRDNAKNTVYLQMNSLKPEDTAVYYCAVEKVYARSWFFHLCSELDFGSWGQGTQVTVSS

SEQ ID NO: 21 - protein, 123 aa, synthetic construct
QVQLVESGGGLVQAGGSLRLSCAASGRTFTNYAMGWFRQAPGKEREFVAAISWSGGGTNYADSVKGRFTISRDNAKNTVYLQMNSPKPEDTAVYYCATTLGYYGSSSRLYEYWGQGTQVTVSS

SEQ ID NO: 22 - protein, 126 aa, synthetic construct
QVQLVESGGGLAQAGGSLRLSCATSGLTFSMSAMGWFRQAPGNEREFVGVINWNGGSSRYADSVRGRFTISRDNAKNTVYLQMNSLKPEDTGVYYCAADALNIGTGEVNPRLYDYWGQGTQVMVSS

SEQ ID NO: 23 - protein, 120 aa, synthetic construct
QVQLVESGGGLVQPGGSLRLSCVASGSTISINSMGWYRQAPGKAREMVATITNDGPIKYADPVKGRFTISRDNPKNTVYLQMNSLKPEDTAVYYCVADRAYWLAGEWEYWGQGTQVTVSS

SEQ ID NO: 24 - protein, 117 aa, synthetic construct
QVQLVESGGGLVQAGGSLRLSCTASGNIFSVNVMGWHRQAPGKRRELVAALTNDGRRNYADSVKGRFTISRDNAKNTIYLEMNSLKPEDTAVYYCGTGTLESGFNSWGQGLQVTVSS

SEQ ID NO: 25-30 - skipped (6 entries)

SEQ ID NO: 31 - protein, 5 aa, synthetic construct
YYAIA

SEQ ID NO: 32 - protein, 5 aa, synthetic construct
ENVMG

SEQ ID NO: 33 - protein, 5 aa, synthetic construct
KDTMG

SEQ ID NO: 34 - protein, 5 aa, synthetic construct
IYAVG

SEQ ID NO: 35 - protein, 5 aa, synthetic construct
KYMTG

SEQ ID NO: 36 - protein, 5 aa, synthetic construct
LNSIG

SEQ ID NO: 37 - protein, 5 aa, synthetic construct
GSAMG

SEQ ID NO: 38 - protein, 5 aa, synthetic construct
INAMA

SEQ ID NO: 39 - protein, 5 aa, synthetic construct
TYAVG

SEQ ID NO: 40 - protein, 5 aa, synthetic construct
INSMG

SEQ ID NO: 41 - protein, 5 aa, synthetic construct
IYGVG

SEQ ID NO: 42 - protein, 5 aa, synthetic construct
NLTLG

SEQ ID NO: 43 - protein, 5 aa, synthetic construct
YYAVG

SEQ ID NO: 44 - protein, 5 aa, synthetic construct
LNSIG

SEQ ID NO: 45 - protein, 5 aa, synthetic construct
INAMA

SEQ ID NO: 46 - protein, 5 aa, synthetic construct
INAMG

SEQ ID NO: 47 - protein, 5 aa, synthetic construct
RSAMG

SEQ ID NO: 48 - protein, 5 aa, synthetic construct
RYIMG

SEQ ID NO: 49 - protein, 5 aa, synthetic construct
IYSVG

SEQ ID NO: 50 - protein, 5 aa, synthetic construct
NYAIG

SEQ ID NO: 51 - protein, 5 aa, synthetic construct
NYAMG

SEQ ID NO: 52 - protein, 5 aa, synthetic construct
MSAMG

SEQ ID NO: 53 - protein, 5 aa, synthetic construct
INSMG

SEQ ID NO: 54 - protein, 5 aa, synthetic construct
VNVMG

SEQ ID NO: 55-60 - skipped (6 entries)

SEQ ID NO: 61 - protein, 17 aa, synthetic construct
CFCSSDGDIRYADSVKG

SEQ ID NO: 62 - protein, 17 aa, synthetic construct
AINWNTGSTYYSDSRKG

SEQ ID NO: 63 - protein, 17 aa, synthetic construct
VISASGDSTYYADFVKG

SEQ ID NO: 64 - protein, 17 aa, synthetic construct
GITWSGGTTYYSDSVKG

SEQ ID NO: 65 - protein, 17 aa, synthetic construct
SIDWSSTTTHYANSVKG

SEQ ID NO: 66 - protein, 17 aa, synthetic construct
CISPSGSRMHYADSVKG

SEQ ID NO: 67 - protein, 16 aa, synthetic construct
VISWNGRTTYADSVKG

SEQ ID NO: 68 - protein, 16 aa, synthetic construct
AITTAGTTNYADSVKG

SEQ ID NO: 69 - protein, 17 aa, synthetic construct
AITGSGRNPYYPDSLKG

SEQ ID NO: 70 - protein, 16 aa, synthetic construct
GIVSDGRINYADSVKG

SEQ ID NO: 71 - protein, 17 aa, synthetic construct
LITWSGGTTYYSDSVKG

SEQ ID NO: 72 - protein, 18 aa, synthetic construct
STSWPDDEMTYYTDSVKG

SEQ ID NO: 73 - protein, 17 aa, synthetic construct
AISWSGRNTNYADSVTG

SEQ ID NO: 74 - protein, 17 aa, synthetic construct
CISPSASSTHYADSVKG

SEQ ID NO: 75 - protein, 16 aa, synthetic construct
AITTSGTTNYADSVKG

SEQ ID NO: 76 - protein, 16 aa, synthetic construct
SISHDGETKYANSVKG

SEQ ID NO: 77 - protein, 22 aa, synthetic construct
GISRRNSGSTYVADSYEDSVKG

SEQ ID NO: 78 - protein, 17 aa, synthetic construct
AINWRGDSTYYADSVKG

SEQ ID NO: 79 - protein, 17 aa, synthetic construct
GITWSGGTTYYSDSVKG

SEQ ID NO: 80 - protein, 17 aa, synthetic construct
CISSSDGDTQYADSVKG

SEQ ID NO: 81 - protein, 17 aa, synthetic construct
AISWSGGGTNYADSVKG

SEQ ID NO: 82 - protein, 17 aa, synthetic construct
VINWNGGSSRYADSVRG

SEQ ID NO: 83 - protein, 16 aa, synthetic construct
TITNDGPIKYADPVKG

SEQ ID NO: 84 - protein, 16 aa, synthetic construct
ALTNDGRRNYADSVKG

SEQ ID NO: 85-90 - skipped (6 entries)

SEQ ID NO: 91 - protein, 20 aa, synthetic construct
ERVYSSTWFFHRYSELDFGS

SEQ ID NO: 92 - protein, 15 aa, synthetic construct
TSSSYYYIGEFIPDY

SEQ ID NO: 93 - protein, 20 aa, synthetic construct
DGARAYYSINAHRYSWAYNY

SEQ ID NO: 94 - protein, 14 aa, synthetic construct
DTYSMTLSARSYDY

SEQ ID NO: 95 - protein, 17 aa, synthetic construct
HQDSSYGYTILPIDYDS

SEQ ID NO: 96 - protein, 17 aa, synthetic construct
GSPSYHYCSMYGLEYDS

SEQ ID NO: 97 - protein, 17 aa, synthetic construct
DHLNYGSGDVNPRTYDY

SEQ ID NO: 98 - protein, 11 aa, synthetic construct
WAGTRDDLNEY

SEQ ID NO: 99 - protein, 14 aa, synthetic construct
RRGAIATMPLDYDH

SEQ ID NO: 100 - protein, 12 aa, synthetic construct
DRAFVYAGDYDY

SEQ ID NO: 101 - protein, 14 aa, synthetic construct
DTYSMTMSARDYEY

SEQ ID NO: 102 - protein, 15 aa, synthetic construct
ASWSGRSTLSYDYDY

SEQ ID NO: 103 - protein, 17 aa, synthetic construct
DYWNYGSGSEVPNNYDT

SEQ ID NO: 104 - protein, 17 aa, synthetic construct
GTPSYHYCSMYGLEYDD

SEQ ID NO: 105 - protein, 11 aa, synthetic construct
WAGTRDDLNEY

SEQ ID NO: 106 - protein, 13 aa, synthetic construct
DVAWVVVAGEYDY

SEQ ID NO: 107 - protein, 17 aa, synthetic construct
EPTLGWYVPRRSVEYEY

SEQ ID NO: 108 - protein, 16 aa, synthetic construct
DRGESYYYTRSTEYTY

SEQ ID NO: 109 - protein, 14 aa, synthetic construct
DRYSMTMSARDYDY

SEQ ID NO: 110 - protein, 20 aa, synthetic construct
EKVYARSWFFHLCSELDFGS

SEQ ID NO: 111 - protein, 14 aa, synthetic construct
TLGYYGSSSRLYEY

SEQ ID NO: 112 - protein, 17 aa, synthetic construct
DALNIGTGEVNPRLYDY

SEQ ID NO: 113 - protein, 12 aa, synthetic construct
DRAYWLAGEWEY

SEQ ID NO: 114 - protein, 9 aa, synthetic construct
GTLESGFNS

SEQ ID NO: 115-120 - skipped (6 entries)

SEQ ID NO: 121 - protein, 30 aa, synthetic construct
QVQLVESGGGLVQPGGSLRLSCAASGITLD

SEQ ID NO: 122 - protein, 30 aa, synthetic construct
QVLLVESGGGLVQAGGLLRLACSASGRTFT

SEQ ID NO: 123 - protein, 30 aa, synthetic construct
QVQLVESGGGLVQAGGSLRLSCAASGRAFS

SEQ ID NO: 124 - protein, 30 aa, synthetic construct
QVQLVESGGGLVQAGGSLRLSCAASGRTFS

SEQ ID NO: 125 - protein, 30 aa, synthetic construct
QVQLVESGGGLVQAGGSLRLSCAASGRTVN

SEQ ID NO: 126 - protein, 30 aa, synthetic construct
QVQLVESGGGLVQPGGSLRLSCAASGFTLD

SEQ ID NO: 127 - protein, 30 aa, synthetic construct
QVQLVESGGGLAQAGGSLRLSCASSGLIFS

SEQ ID NO: 128 - protein, 30 aa, synthetic construct
QVQLVESGGGLVQPGGSLRLSCARSENNFS

SEQ ID NO: 129 - protein, 30 aa, synthetic construct
QVQLVESGGGAVQAGDSLRLSCTASGRTFS

SEQ ID NO: 130 - protein, 30 aa, synthetic construct
QVQLVESGGGLVQAGGSLRLSCEVSGSIYS

SEQ ID NO: 131 - protein, 30 aa, synthetic construct
QVQLVESGGGLVQAGGSLRLSCAASGRTSS

SEQ ID NO: 132 - protein, 30 aa, synthetic construct
QVQLVESGGRLVQAGGSLRLSCAAPGRTFR

SEQ ID NO: 133 - protein, 30 aa, synthetic construct
QVQLVESGGGLVQAGGSLRLSCAASGRTVG

SEQ ID NO: 134 - protein, 30 aa, synthetic construct; identical to SEQ ID NO: 126

SEQ ID NO: 135 - protein, 30 aa, synthetic construct; identical to SEQ ID NO: 128

SEQ ID NO: 136 - protein, 30 aa, synthetic construct
QVQLVESGGGLVEAGGSLRLSCVASGSSSS

SEQ ID NO: 137 - protein, 30 aa, synthetic construct
QVQLVESGGGLVQAGGSLRLSCSASGGTAS

SEQ ID NO: 138 - protein, 30 aa, synthetic construct
QVQLVESGGGLVQAGGSLRLSCAASGRTGS

SEQ ID NO: 139 - protein, 30 aa, synthetic construct; identical to SEQ ID NO: 131

SEQ ID NO: 140 - protein, 30 aa, synthetic construct; identical to SEQ ID NO: 126

SEQ ID NO: 141 - protein, 30 aa, synthetic construct
QVQLVESGGGLVQAGGSLRLSCAASGRTFT

SEQ ID NO: 142 - protein, 30 aa, synthetic construct
QVQLVESGGGLAQAGGSLRLSCATSGLTFS

SEQ ID NO: 143 - protein, 30 aa, synthetic construct
QVQLVESGGGLVQPGGSLRLSCVASGSTIS

SEQ ID NO: 144 - protein, 30 aa, synthetic construct
QVQLVESGGGLVQAGGSLRLSCTASGNIFS

SEQ ID NO: 145 - protein, 30 aa, synthetic construct
  VARIANT 3: note = Q or L
  VARIANT 10: note = G or R
  VARIANT 11: note = L or A
  VARIANT 12: note = V or A
  VARIANT 14: note = P or A
  VARIANT 16: note = G or D
  VARIANT 17: note = S or L
  VARIANT 21: note = S or A
  VARIANT 23: note = A, S, T, E, or V
  VARIANT 24: note = A, S, R, V, or T
  VARIANT 25: note = S or P
  VARIANT 26: note = G or E
  VARIANT 27: note = I, R, F, L, N, S, or G
  VARIANT 28: note = T, A, I, N, or S
  VARIANT 29: note = L, F, V, Y, S, A, G, or I
  VARIANT 30: note = D, T, S, N, R, or G
QVXLVESGGXXXZXGXXLRLXCXXXXXXXX

SEQ ID NO: 146 - protein, 30 aa, synthetic construct; identical to SEQ ID NO: 124

SEQ ID NO: 147-150 - skipped (4 entries)

SEQ ID NO: 151 - protein, 14 aa, synthetic construct
WFRQAPGKEREGVS

SEQ ID NO: 152 - protein, 14 aa, synthetic construct
WFRQAPGKEREFVA

SEQ ID NO: 153 - protein, 14 aa, synthetic construct
WFRQAPGKEREFVA

SEQ ID NO: 154 - protein, 14 aa, synthetic construct
WFRQAPGKERDFVA

SEQ ID NO: 155 - protein, 14 aa, synthetic construct
WFRQTPGKEREFVA

SEQ ID NO: 156 - protein, 14 aa, synthetic construct
WFRQAPGKEREGVS

SEQ ID NO: 157 - protein, 14 aa, synthetic construct
WFRQAPGKEREFVG

SEQ ID NO: 158 - protein, 14 aa, synthetic construct
WYRQAPGKQRELVA

SEQ ID NO: 159 - protein, 14 aa, synthetic construct
WFRQAPGKERELLA

SEQ ID NO: 160 - protein, 14 aa, synthetic construct
WYRQAPGKQRELVA

SEQ ID NO: 161 - protein, 14 aa, synthetic construct
WFRQAPGKERELVA

SEQ ID NO: 162 - protein, 14 aa, synthetic construct
WFRQAPGKEREFVA

SEQ ID NO: 163 - protein, 14 aa, synthetic construct
WFRQAPGKERDFVA

SEQ ID NO: 164 - protein, 14 aa, synthetic construct
WFRQAPGKEREGVS

SEQ ID NO: 165 - protein, 14 aa, synthetic construct
WYRQAPGKQRELVA

SEQ ID NO: 166 - protein, 14 aa, synthetic construct
WYRQAPGKQRELVA

SEQ ID NO: 167 - protein, 14 aa, synthetic construct
WFRQAPGKEREFVA

SEQ ID NO: 168 - protein, 14 aa, synthetic construct
WFRQAPGKEREFVA

SEQ ID NO: 169 - protein, 14 aa, synthetic construct
WFRQAPGKERELVA

SEQ ID NO: 170 - protein, 14 aa, synthetic construct
WFRQAPGKEREGVS

SEQ ID NO: 171 - protein, 14 aa, synthetic construct
WFRQAPGKEREFVA

SEQ ID NO: 172 - protein, 14 aa, synthetic construct
WFRQAPGNEREFVG

SEQ ID NO: 173 - protein, 14 aa, synthetic construct
WYRQAPGKAREMVA

SEQ ID NO: 174 - protein, 14 aa, synthetic construct
WHRQAPGKRRELVA

SEQ ID NO: 175 - protein, 14 aa, synthetic construct
  VARIANT 2: note = F, Y, or H
  VARIANT 5: note = A or T
  VARIANT 8: note = K or N
  VARIANT 9: note = E, Q, A, or R
  VARIANT 11: note = E or D
  VARIANT 13: note = V or L
  VARIANT 14: note = S, A, or G
  VARIANT 12: note = G, F, L, or M
WXRQXPGXXRXXXX

SEQ ID NO: 176 - protein, 14 aa, synthetic construct
WFRQAPGKEREFVA

SEQ ID NO: 177-180 - skipped (4 entries)

SEQ ID NO: 181 - protein, 32 aa, synthetic construct
RFTISRDNAKNTVYLQMNSLKPEDTAVYYCAT

SEQ ID NO: 182 - protein, 32 aa, synthetic construct
RFTISKDNAKNTMFLQINNLKPEDTAVYYCGG

SEQ ID NO: 183 - protein, 32 aa, synthetic construct
RFTISRDNAKNTEYLQMNSLKPEDTAVYHCAA

SEQ ID NO: 184 - protein, 32 aa, synthetic construct
RFTISRDNAKNTVYLQMNNLKPEDTAIYYCAV

SEQ ID NO: 185 - protein, 32 aa, synthetic construct
RFTISRDKVKNTVYLQMNGLKPEDTAVYYCAA

SEQ ID NO: 186 - protein, 32 aa, synthetic construct; identical to SEQ ID NO: 181

SEQ ID NO: 187 - protein, 32 aa, synthetic construct
RFTISRDDAVNMVWLQMNNLKPEDTAVYYCAA

SEQ ID NO: 188 - protein, 32 aa, synthetic construct
RFTISRDNAKTTIYLQMNSLKPEDTAVYYCNL

SEQ ID NO: 189 - protein, 32 aa, synthetic construct
RFTISREYAKLPVYLQMNSLKPEDTAVYYCAA

SEQ ID NO: 190 - protein, 30 aa, synthetic construct
RFTISRAKNTVYLQMNSLKPEDTAVYYCAA

SEQ ID NO: 191 - protein, 32 aa, synthetic construct
RFTISKDNAKNTVFLQMNNLKPEDTAIYYCAV

SEQ ID NO: 192 - protein, 32 aa, synthetic construct
RFTISRDNAKNTVYLQMNSLKPEDTAVYYCAA

SEQ ID NO: 193 - protein, 32 aa, synthetic construct
RFTIFRDNAKNTVDLQMNSLKPEDTAVYYCAA

SEQ ID NO: 194 - protein, 32 aa, synthetic construct; identical to SEQ ID NO: 181

SEQ ID NO: 195 - protein, 32 aa, synthetic construct; identical to SEQ ID NO: 188

SEQ ID NO: 196 - protein, 32 aa, synthetic construct
RFTISRGNAKNTIYLQMNSLKPEDTAVYYCVG

SEQ ID NO: 197 - protein, 32 aa, synthetic construct
RFTISRDNAKNTIYLQMNSLKPEDTAVYYCAA

SEQ ID NO: 198 - protein, 32 aa, synthetic construct
RFTIARDNAKNTAYLQMNSLKPEDTAVYYCVA

SEQ ID NO: 199 - protein, 32 aa, synthetic construct
RFTISRDNAKNTVNLQMNNLKPEDTAIYYCAV

SEQ ID NO: 200 - protein, 32 aa, synthetic construct
RFTISRDNAKNTVYLQMNSLKPEDTAVYYCAV

SEQ ID NO: 201 - protein, 32 aa, synthetic construct
RFTISRDNAKNTVYLQMNSPKPEDTAVYYCAT

SEQ ID NO: 202 - protein, 32 aa, synthetic construct
RFTISRDNAKNTVYLQMNSLKPEDTGVYYCAA

SEQ ID NO: 203 - protein, 32 aa, synthetic construct
RFTISRDNPKNTVYLQMNSLKPEDTAVYYCVA

SEQ ID NO: 204 - protein, 32 aa, synthetic construct
RFTISRDNAKNTIYLEMNSLKPEDTAVYYCGT

SEQ ID NO: 205 - protein, 32 aa, synthetic construct
  VARIANT 5: note = S, F, or A
  VARIANT 6: note = R or K
  VARIANT 7: note = D, E, G, or no residue
  VARIANT 8: note = N, K, D, Y, or no residue
  VARIANT 9: note = A, V, or P
  VARIANT 10: note = K or V
  VARIANT 12: note = T, M, or P
  VARIANT 13: note = V, M, E, I, or A
  VARIANT 14: note = Y, F, W, D, or N
  VARIANT 17: note = M or I
  VARIANT 19: note = S, N, or G
  VARIANT 20: note = L or P
  VARIANT 26: note = A or G
  VARIANT 27: note = V or I
  VARIANT 29: note = Y or H
  VARIANT 31: note = A, G, N, or V
  VARIANT 32: note = T, G, A, V, or L
  VARIANT 11: note = N, T, or L
RFTIXXXXXXXXXXLZXNXXKPEDTXXYXCXX

SEQ ID NO: 206 - protein, 32 aa, synthetic construct; identical to SEQ ID NO: 192

SEQ ID NO: 207-210 - skipped (4 entries)

SEQ ID NO: 211 - protein, 11 aa, synthetic construct
WGRGTQVTVSS

SEQ ID NO: 212 - protein, 11 aa, synthetic construct
WGQGTQVTVSS

SEQ ID NO: 213 - protein, 11 aa, synthetic construct
WGQGTQVTVSS

SEQ ID NO: 214 - protein, 11 aa, synthetic construct
WGQGTQVTVSS

SEQ ID NO: 215 - protein, 11 aa, synthetic construct
WGQGTQVTVSS

SEQ ID NO: 216 - protein, 11 aa, synthetic construct
WGQGTQVTVPS

SEQ ID NO: 217 - protein, 11 aa, synthetic construct
WGQGTQVTVSS

SEQ ID NO: 218 - protein, 11 aa, synthetic construct
WGQGTQVTVSS

SEQ ID NO: 219 - protein, 11 aa, synthetic construct
WGQGTQVTVSS

SEQ ID NO: 220 - protein, 11 aa, synthetic construct
LGQGTQVTVSS

SEQ ID NO: 221 - protein, 11 aa, synthetic construct
WGQGTQVTVSS

SEQ ID NO: 222 - protein, 11 aa, synthetic construct
WGQGTQVTVST

SEQ ID NO: 223 - protein, 11 aa, synthetic construct
WGQGTQVTVSS

SEQ ID NO: 224 - protein, 11 aa, synthetic construct
WGQGTQVTVSS

SEQ ID NO: 225 - protein, 11 aa, synthetic construct
WGQGTQVTVSS

SEQ ID NO: 226 - protein, 11 aa, synthetic construct
WGQGTQVTVSS

SEQ ID NO: 227 - protein, 11 aa, synthetic construct
WGQGTQVTVSS

SEQ ID NO: 228 - protein, 11 aa, synthetic construct
WGQGTQVTVSS

SEQ ID NO: 229 - protein, 11 aa, synthetic construct
WGQGTQVTVSS

SEQ ID NO: 230 - protein, 11 aa, synthetic construct
WGQGTQVTVSS

SEQ ID NO: 231 - protein, 11 aa, synthetic construct
WGQGTQVTVSS

SEQ ID NO: 232 - protein, 11 aa, synthetic construct
WGQGTQVMVSS

SEQ ID NO: 233 - protein, 11 aa, synthetic construct
WGQGTQVTVSS

SEQ ID NO: 234 - protein, 11 aa, synthetic construct
WGQGLQVTVSS

SEQ ID NO: 235 - protein, 11 aa, synthetic construct
  VARIANT 1: note = W or L
  VARIANT 3: note = R or Q
  VARIANT 5: note = T or L
  VARIANT 8: note = T or M
  VARIANT 10: note = S or P
  VARIANT 11: note = S or T
XGXGXQVXVXX

SEQ ID NO: 236 - protein, 11 aa, synthetic construct
WGQGTQVTVSS

SEQ ID NO: 237-239 - skipped (3 entries)

SEQ ID NO: 240 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 241 - protein, 12 aa, synthetic construct
NRKRISNCVADY

SEQ ID NO: 242 - protein, 5 aa, synthetic construct
FTNVY

SEQ ID NO: 243 - protein, 5 aa, synthetic construct
KPFER

SEQ ID NO: 244 - protein, 7 aa, synthetic construct
ATVCGPK

SEQ ID NO: 245 - protein, 7 aa, synthetic construct
VRDPQTL

SEQ ID NO: 246 - protein, 45 aa, synthetic construct
LVKQLSSNFGAISSVLNDILSRLDKVEAEVQIDRLITGRLQSLQT

SEQ ID NO: 247 - protein, 17 aa, synthetic construct
MQMAYRFNGIGVTQNVL

SEQ ID NO: 248 - protein, 34 aa, synthetic construct
EPQIITTDNFVSGNCDVVIGIVNNTVYDPLQPEL

SEQ ID NO: 249 - protein, 14 aa, synthetic construct
DRLNEVAKNLNESL

SEQ ID NO: 250 - skipped

SEQ ID NO: 251 - protein, 8 aa, synthetic construct
HHHHHHHH

SEQ ID NO: 252 - protein, 4 aa, synthetic construct
ASVG

SEQ ID NO: 253 - protein, 4 aa, synthetic construct
RSVR

SEQ ID NO: 254 - protein, 4 aa, synthetic construct
GSAS

SEQ ID NO: 255 - protein, 4 aa, synthetic construct
RRAR

SEQ ID NO: 256 - protein, 5 aa, synthetic construct
GGGGS